SEQUENCE LISTING (28 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 18 aa, Homo sapiens
QEFEVMEDHAGTYGLGDR

SEQ ID NO: 3 - protein, 21 aa, Homo sapiens
KDQGGYTMHQDQEGDTDAGLK

SEQ ID NO: 4 - protein, 20 aa, Homo sapiens
  MOD_RES (7)..(7): M is oxidized methionine
DQGGYTMHQDQEGDTDAGLK

SEQ ID NO: 5 - protein, 23 aa, Homo sapiens
ESPLQTPTEDGSEEPGSETSDAK

SEQ ID NO: 6 - protein, 20 aa, Homo sapiens
STPTAEDVTAPLVDEGAPGK

SEQ ID NO: 7 - protein, 39 aa, Homo sapiens
QAAAQPHTEIPEGTTAEEAGIGDTPSLEDEAAGHVTQAR

SEQ ID NO: 8 - protein, 8 aa, Homo sapiens
GAAPPGQK

SEQ ID NO: 9 - protein, 10 aa, Homo sapiens
TPPSSGEPPK

SEQ ID NO: 10 - protein, 15 aa, Homo sapiens
SGYSSPGSPGTPGSR

SEQ ID NO: 11 - protein, 10 aa, Homo sapiens
TPSLPTPPTR

SEQ ID NO: 12 - protein, 13 aa, Homo sapiens
TPSLPTPPTREPK

SEQ ID NO: 13 - protein, 12 aa, Homo sapiens
  MOD_RES (8)..(8): M is oxidized methionine
LQTAPVPMPDLK

SEQ ID NO: 14 - protein, 8 aa, Homo sapiens
IGSTENLK

SEQ ID NO: 15 - protein, 10 aa, Homo sapiens
KLDLSNVQSK

SEQ ID NO: 16 - protein, 9 aa, Homo sapiens
LDLSNVQSK

SEQ ID NO: 17 - protein, 19 aa, Homo sapiens
HVPGGGSVQIVYKPVDLSK

SEQ ID NO: 18 - protein, 16 aa, Homo sapiens
IGSLDNITHVPGGGNK

SEQ ID NO: 19 - protein, 17 aa, Homo sapiens
IGSLDNITHVPGGGNKK

SEQ ID NO: 20 - protein, 10 aa, Homo sapiens
TDHGAEIVYK

SEQ ID NO: 21 - protein, 11 aa, Homo sapiens
SPVVSGDTSPR

SEQ ID NO: 22 - protein, 32 aa, Homo sapiens
  MOD_RES (13)..(13): M is oxidized methionine
HLSNVSSTGSIDMVDSPQLATLADEVSASLAK

SEQ ID NO: 23 - protein, 24 aa, Homo sapiens
AEEAGIGDTPSLEDEAAGHVTQAR

SEQ ID NO: 24 - protein, 30 aa, Homo sapiens
STPTAEAEEAGIGDTPSLEDEAAGHVTQAR

SEQ ID NO: 25 - protein, 12 aa, Homo sapiens
VQIVYKPVDLSK

SEQ ID NO: 26 - protein, 19 aa, Homo sapiens
HVPGGGSVQIVYKPVDLSK

SEQ ID NO: 27 - protein, 23 aa, Homo sapiens
ESPLQTPTEDGSEEPGSETSDAK

SEQ ID NO: 28 - protein, 20 aa, Homo sapiens
STPTAEDVTAPLVDEGAPGK

(19) United States
(12) Patent Application Publication
(10) Pub. No.: US 2025/0298039 A1
(43) Pub. Date: Sep. 25, 2025

(54) METHODS AND COMPOSITIONS FOR TAUOPATHY DIAGNOSIS AND TREATMENT

(71) Applicant: Children`s Medical Center Corporation, Boston, MA (US)

(72) Inventors: Judith AJ Steen, Brighton, MA (US); Hanno Steen, Brighton, MA (US); Hendrik Wesseling, Boston, MA (US); Mukesh Kumar, Brookline, MA (US); Waltraud Mair, Mannheim, Baden-Württemberg (DE); Pieter Beerepoot, Boston, MA (US)

(21) Appl. No.: 18/036,020

(22) PCT Filed: Nov. 12, 2021
(86) PCT No.: PCT/US2021/059240

§ 371 Date: May 9, 2023

Related U.S. Application Data
(60) Provisional application No. 63/113,118, filed on Nov. 12, 2020.

(51) Int. Cl. G01N 33/68

(52) U.S. Cl. CPC G01N 33/6896; G01N 2333/4709; G01N 2440/10; G01N 2440/12; G01N 2440/14; G01N 2440/36; G01N 2800/2821; G01N 2800/56

(57) ABSTRACT

This disclosure relates to methods for diagnosing and treating a tauopathy, e.g., Alzheimer's disease, in a subject, the methods comprising, in part, identifying one or more post-translation modifications (PTMs) in the subject.

### STATEMENT OF FEDERALLY SPONSORED RESEARCH

This invention was made with government support under grant numbers AG019724, AG023501, GM096319, GM112007, and NS066973, and contract number HSSN271201300030C, awarded by the National Institutes of Health. The government has certain rights in the invention.

### SEQUENCE LISTING

This application contains a Sequence Listing that has been submitted electronically as an ASCII text file named SequenceListing_ST25.txt. The ASCII text file, created on May 2, 2023, is 10,342 bytes in size. The material in the ASCII text file is hereby incorporated by reference in its entirety.

### CROSS REFERENCE

This application is the national stage entry of International Patent Application No. PCT/US2021/059240, filed on Nov. 12, 2021, which claims the benefit of U.S. Provisional Patent Application Ser. No. 63/113,118, filed on Nov. 12, 2020; the entire content of which is hereby incorporated by reference in its entirety.

### TECHNICAL FIELD

This disclosure relates to diagnosing and treating tauopathies, such as Alzheimer's disease (AD).

### BACKGROUND

Alzheimer's disease (AD) is associated with aging, results in devastating disability, diminished quality of life and its occurrence will reach epidemic proportions by 2050 if unabated. The pathological hallmarks of AD are the two proteins amyloid beta (Aβ) and Tau. Aβ forms extracellular plaques (Glenner and Wong, 1984; Masters et al., 1985), whereas Tau forms intracellular neurofibrillary tangles (Grundke-Iqbal et al., 1986), (Kosik et al., 1986), (Wood et al., 1986).

During disease progression in AD, pathological neurofibrillary Tau aggregates show a pattern of accumulation which starts in the entorhinal cortex and spreads through connected pathways to cortical areas (Jucker and Walker, 2013). Given that a protein can be post translationally modified (PTM), which gives rise to different proteoforms (Smith et al., 2013), the combinations of such modifications are responsible for regulating and fine-tuning protein conformation and activity (Aebersold et al., 2018; Soria et al., 2014) and can dramatically change the function and toxicity of proteins. To effectively understand the functional significance of protein modification, all possible PTMs should be mapped from N- to C-terminus of a protein. While several modifications are present at basal levels in cells, not all of these are significant in the context of specific pathological conditions (Singh et al., 2014). Thus, profiling the specific molecular characteristics of pathological Tau in human AD is critical to the early diagnosis and development of mechanism directed therapies. It is also important to obtain information about the extent of modification to understand the function of a particular PTM. While tau in the normal brain contains 2-3 phosphorylated residues per tau molecule, it is estimated to be approximately 3-fold hyper-phosphorylated in AD brain. Accumulating data indicates that phosphorylation alone is not sufficient for aggregation and might even serve a protective role. Several other Post-Translational Modifications (PTMs) such as acetylation, ubiquitination, methylation, and glycosylation, among others, appear to play regulatory roles as well with respect to rates of tau clearance and aggregation and thus contribute to tau pathology.

Currently, it is not known what the minimum set of PTMs is that is important for oligomer formation and Tau aggregation, and it is not known which PTMs occur on Tau seeds that can potentiate further aggregation. The mechanistic role of PTMs in initiating Tau seeding and propagation still remains to be elucidated (Guo et al., 2017)(Wang and Mandelkow, 2016). Immunohistochemistry has been the workhorse for the identification and quantification of Tau proteoforms. While such antibody-based approaches simple to implement and provide semiquantitative information, they cannot measure quantitative changes accurately or determine PTM stoichiometry. Finally, all antibody-based approaches require a-priori knowledge of sites and therefore cannot be used for discovery purposes. Thus, there is a need to develop an assay to determine the PTM of the tau protein, and identify the pathological PTM in tauopathies such as Alzheimer's disease for developing diagnosis and treatment.

### SUMMARY

This disclosure relates to diagnosing and treating tauopathies.

In one aspect, provided herein are methods for diagnosing a tauopathy in a subject, the method comprising: (a) obtaining a biological sample from the subject; (b) identifying one or more post translational modifications (PTMs) in a tau protein, wherein the one or more PTMs are at positions selected from the group consisting of K24, Y29, T30, T39, K44, S46, S56, S61, S64, K67, S68, T69, T71, K87, T102, T111, S113, T153, T175, K180, T181, S184, S185, S191, S198, S199, S202, T205, S210, T212, S214, T217, T220, T231, S235, S237, S238, K240, S241, K254, K257, S258, K259, S262, T263, K267, K274, K280, K281, S289, K290, S293, K298, S305, Y310, K311, S316, K317, K321, K331, K343, K347, S352, K353, S356, T361, K369, K370, K375, K385, T386, Y394, K395, S396, S400, T403, S404, R406, S409, S412, S413, T414, S416, S422, S433, S435, K436, and K438 (based on numbering on human 2N4R Tau), thereby diagnosing the tauopathy in the subject.

In some embodiments, the post-translational modification is phosphorylation, glycosylation, glycation, prolyl-isomerization, cleavage or truncation, nitration, polyamination, ubiquitination, acetylation, methylation, dimethylation, trimethylation or sumoylation.

In some embodiments, the subject has an overall higher level of PTMs at the one or more PTM positions as compared to a control level.

In some embodiments, the one or more PTM is selected from the group consisting of phosphorylation at one or more positions selected from the group consisting of Y29, T30, T39, S46, S56, S68, T69, T71, T102, T111, S113, T153, T175, T181, S184, S185, S191, S198, S199, S202, T205, S210, T212, S214, T217, T220, T231, S235, S237, S238, S241, S258, S262, T263, S289, S293, S305, Y310, S316, S352, S356, T361, T386, Y394, S396, S400, T403, S404, S409, S412, S413, T414, S422, S433, and S435; acetylation at one or more positions selected from the group consisting of K24, K44, K240, K267, K274, K280, K281, K298, K311, K317, K331, K343, K347, K353, K369, K370, K375, K385, and K395; ubiquitination at one or more positions selected from the group consisting of K180, K240, K254, K257, K259, K267, K274, K281, K290, K298, K311, K317, K321, K343, K353, K369, and K395; and methylation at one or more positions selected from the group consisting of K67, K87, R406, and K438 (all numbering based on human 2N4R isoform).

In some embodiments, the one or more PTMs comprise ubiquitination at K311 and K317.

In some embodiments, the one or more PTMs comprise phosphorylation at T217 and S262.

In some embodiments, the one or more PTMs are located at the proline-rich region (PRR).

In some embodiments, the one or more PTMs are located at amino acid residues 212-221 of a tau protein.

In some embodiments, the one or more PTMs are selected from the group consisting of S212, S214, S217 and T220.

In some embodiments, the one or more PTMs are located at a region that is C-terminus relative to the N-region of the tau protein.

In some embodiments, the one or more PTMs are located at amino acid residues 354-369 of a tau protein.

In some embodiments, the one or more PTMs are located at amino acid residues 407-437 of a tau protein.

In some embodiments, the one or more PTMs comprise phosphorylation at one or more positions selected from the group consisting of S199, S202 and T205.

In some embodiments, the one or more PTMs comprise phosphorylation at one or more positions selected from the group consisting of S198, S199, S202, and T205.

In some embodiments, the one or more PTMs comprise phosphorylation at one or more positions selected from the group consisting of 5212, S214, and S217.

In some embodiments, the one or more PTMs comprise phosphorylation at T181 and/or T231.

In some embodiments, the one or more PTMs comprise acetylation at one or more positions selected from the group consisting of K353, K369, K370, and K375.

In some embodiments, the tau protein is a 2N4R isoform.

In some embodiments, the biological sample is brain tissue, plasma, or cerebrospinal fluid (CSF).

In some embodiments, the biological sample is obtained from an angular gyrus-associated tissue or sample.

In some embodiments, the angular gyrus-associated tissue or sample is a cerebrospinal fluid (CSF) from the subject.

In some embodiments, the angular gyrus-associated tissue or sample is a plasma sample from the subject.

In some embodiments, the biological sample is obtained from a frontal gyrus-associated tissue or sample.

In some embodiments, the frontal gyrus-associated tissue or sample is a cerebrospinal fluid (CSF) from the subject.

In some embodiments, the frontal gyrus-associated tissue or sample is a plasma sample from the subject.

In some embodiments, the tauopathy is Alzheimer's disease (AD).

In some embodiments, the diagnosing further comprises performing an additional test on the subject.

In some embodiments, the additional test is selected from the group consisting of a behavioral test, a neurological exam, a brain imaging, a mental status test, a dementia test, and mood assessment.

In some embodiments, the one or more PTMs are identified by a method selected from the group consisting of kinase activity assays, phospho-specific antibody assays, Western blot, enzyme-linked immunosorbent assays (ELISA), cell-based ELISA, intracellular flow cytometry, mass spectrometry, multi-analyte profiling, methylation-sensitive restriction enzyme digestion, bisulfite treatment and sequencing, and deamination and sequencing.

In some embodiments, PTMs are identified by the method comprising: (i) providing a labeled sample comprising a labeled tau protein; (ii) mixing the biological sample and the labeled sample at an initial mixing ratio of tau protein to labeled tau protein to form a mixture; (iii) subjecting the mixture to proteolytic digestion, generating tau peptide fragments and labeled tau peptide fragments; (iv) quantifying the abundance of the tau peptide fragments and the labeled tau peptide fragments; (v) measuring the ratio of the abundance of the tau peptide fragments and the labeled tau peptide fragments; (vi) determining the amount of the tau PTMs associated with one or more tau peptide fragments by comparing the measured ratio for each tau peptide fragment to the initial mixing ratio, wherein the extent of deviation from the initial mixing ratio indicates the amount of PTMs in the tau peptide fragment;

In some embodiments, the methods provided herein further comprises comparing the amount of tau PTMs associated with one or more tau peptide fragments with one or more reference levels for the tau peptide fragments.

In some embodiments, subjecting the mixture to proteolytic digestion is performed using one or more proteases.

In some embodiments, the one or more proteases are selected from the group consisting of trypsin, Lys-C, Arg-C, Asp-N, Glu-C, Lys-N, thermolysin, elastase, Tryp-N, and chymotrypsin.

In some embodiments, the methods described herein further comprises purifying the tau protein in the biological sample and the labeled tau protein in the labeled sample before mixing the biological sample and the second sample.

In some embodiments, the labeled tau protein is a fusion protein comprising the tau protein conjugated to first member of a binding pair, wherein the binding pair is selected from the group consisting of biotin/streptavidin, biotin/avidin, biotin/neutravidin, biotin/captavidin, epitope/antibody, protein A/immunoglobulin, protein G/immunoglobulin, protein L/immunoglobulin, GST/glutathione, His-tag/Metal (e.g., nickel, cobalt or copper), antigen/antibody, FLAG/M1 antibody, maltose binding protein/maltose, calmodulin binding protein/calmodulin, enzyme-enzyme substrate, and receptor-ligand binding pairs.

In some embodiments, the mixing ratio of labeled tau protein to tau protein is 4:1, 3:1, 2:1, 1:1, 1:2, 1:3 or 1:4.

In some embodiments, the reference sample comprises predetermined, statistically significant reference analyte levels.

In some embodiments, the labeled tau protein is generated from a cell-free expression system in the presence of isotopically labeled amino acids.

In some embodiments, the labeled tau protein comprises one or more isotope-label amino acid residues.

In some embodiments, the isotope is selected from the group consisting of 13C and 15N.

In some embodiments, determining the abundance of the unlabeled tau peptide fragments and the labeled tau peptide fragments comprises identifying an ion signal associated with a peptide and/or its fragment ions.

In some embodiments, the abundance of the tau peptide fragments and the labeled tau peptide fragments are determined by liquid chromatography-selected reaction monitoring (LC-SRM) or Parallel Reaction Monitoring (PRM).

Also provided herein are methods for detecting one or more tau peptide fragments of a tau protein having altered post translational modification (PTM), the method comprising: (a) obtaining a biological sample from the subject; (b) determining the amount of post translational modification (PTM) associated with one or more tau peptide fragments of a tau protein in the biological sample, wherein determining the amount of PTM comprises: (i) providing a labeled sample comprising a labeled tau protein; (ii) mixing the biological sample and the labeled sample at an initial mixing ratio of tau protein to labeled tau protein to form a mixture; (iii) subjecting the mixture to proteolytic digestion, generating tau peptide fragments and labeled tau peptide fragments; (iv) quantifying the abundance of the tau peptide fragments and the labeled tau peptide fragments; (v) measuring the ratio of the abundance of the tau peptide fragments and the labeled tau peptide fragments; (vi) determining the amount of the tau PTMs associated with one or more tau peptide fragments by comparing the measured ratio for each tau peptide fragment to the initial mixing ratio, wherein the extent of deviation from the initial mixing ratio indicates the amount of PTMs in the tau peptide fragment; (c) comparing the amount of the tau PTMs associated with one or more tau peptide fragments with one or more reference levels for the tau peptide fragments; and (b) identifying one or more tau peptide fragments of a tau protein as having one or more altered post translational modification (PTM), wherein the one or more tau peptide is selected from the peptides listed in Table 2, thereby detecting the one or more tau peptide fragments of a tau protein having altered post translational modification (PTM) in the subject.

Also provided herein are methods for treating a tauopathy in a subject, the method comprising: (a) obtaining a biological sample from the subject; (b) identifying one or more post translational modification (PTM) associated with one or more tau peptide fragments of a tau protein in the biological sample, wherein the one or more PTM is located on the position selected from the positions listed in Table 1; (c) administering an effective amount of a therapeutic agent that targets the one or more tau peptide fragments, thereby treating the tauopathy.

Also provided herein are methods for treating a tauopathy in a subject, the method comprising: administering an effective amount of a therapeutic agent that specifically targets a tau peptide having one or more PTM or a tau peptide fragment having one or more PTM, wherein the one or more PTM is located on the position selected from the positions listed in Table 1, thereby treating the tauopathy.

In some embodiments, the one or more PTM is selected from the group consisting of a phosphorylation at T231, a phosphorylation at S235, a phosphorylation at S237, a phosphorylation at S238, a ubiquitination at K311 and a ubiquitination at K317. In some embodiments, the one or more PTM is a phosphorylation at T231 and S235. In some embodiments, the one or more PTM is a phosphorylation at T231 and S237. In some embodiments, the one or more PTM is a phosphorylation at T231 and S238. In some embodiments, the one or more PTM is a ubiquitination at K311. In some embodiments, the one or more PTM is a ubiquitination at K317. In some embodiments, the one or more PTM is a ubiquitination at K311 and a ubiquitination at K317.

In some embodiments, the therapeutic agent is an antibody or antigen-binding fragment thereof that binds to a Tau peptide.

In some embodiments, the antibody is selected from the antibodies listed in Table 3, or wherein the antibody has the one or more CDR(s) comprising an amino acid sequence that is at least 80% identical to one or more CDR(s) of any one of the antibodies listed in Table 3.

In some embodiments, the therapeutic agent reduces or eliminates the seeding of the Tau peptide.

Also provided herein are methods for determining the efficacy of a treatment of a tauopathy in a subject, the method comprising: (a) obtaining a biological sample from the subject; (b) identifying one or more post translational modification (PTM) associated with one or more tau peptide fragments of a tau protein in the biological sample after one or more treatment of a tauopathy, wherein the one or more PTM is located on the position selected from the positions listed in Table 1.

Also provided herein are methods for determining a progression of a tauopathy in a subject, the method comprising: (a) obtaining a biological sample from the subject; (b) identifying one or more post translational modification (PTM) associated with one or more tau peptide fragments of a tau protein in the biological sample, wherein the one or more PTMs are located at positions selected from: (i) one or more of T181, T231, S235, S400, T403, and S404; (ii) one or more of S199, 5202, T212, T217, S237, S262, and S396; (iii) one or more of T175, S210, S214, K254, K259, T263, K267, K274, K281, S289, K290, S305, K311, K317, K321, K353, and K369; or (iv) one or more of S55, S113, T153, S191, S198, T205, K257, S5293, K370, and K375.

In some embodiments, the PTMs identified in (i) and (ii), and not (iii) and (iv) indicate an early stage of a tauopathy.

In some embodiments, the PTMs identified in (i) correspond to Braak 0-III stage of Alzheimer's disease.

In some embodiments, the PTMs identified in (iii) and (iv) indicate a late stage of a tauopathy.

In some embodiments, the PTMs identified in (iii) correspond to Braak-V stage of Alzheimer's disease.

Also provided herein are methods for determining stage of a tauopathy in a subject, the method comprising: (a) obtaining a biological sample from the subject; (b) identifying one or more post translational modification (PTM) associated with one or more tau peptide fragments of a tau protein in the biological sample, wherein the one or more PTMs are located at positions selected from: (i) one or more PTMs in S210, S289, K274 (in 3R isoform of tau protein), K321, S305, K311 (in 3R isoform of tau protein); (ii) one or more PTMs in T175, S237, and K281; (iii) one or more PTMs in S235, T181, T231, T403, S404, S400, S262, T217, S396, and T212; (iv) one or more PTMs in S214, K311, K353, K267, K259, K317 (in 3R isoform of tau protein), K311 (in 3R isoform of tau protein), K311, K317, K254, K369, S262, and T263; and (v) one or more PTMs in S199 and S202.

In one aspect, the disclosure relates to a method of reducing or eliminating a seeding activity of a Tau peptide, or reducing the risk of tau aggregation propagation, the method comprising: contacting a tau peptide or a tau peptide fragment with a therapeutic agent that targets a tau peptide having one or more PTM or a tau peptide fragment having one or more PTM.

In some embodiments, the one or more PTM is selected from the group consisting of a phosphorylation at T231, a phosphorylation at S235, a phosphorylation at S237, a phosphorylation at S238, a ubiquitination at K311 and a ubiquitination at K317.

In some embodiments, the one or more PTM is a phosphorylation at T231 and S235. In some embodiments, the one or more PTM is a phosphorylation at T231 and S237. In some embodiments, the one or more PTM is a phosphorylation at T231 and S238. In some embodiments, the one or more PTM is a ubiquitination at K311. In some embodiments, the one or more PTM is a ubiquitination at K317. In some embodiments, the one or more PTM is a ubiquitination at K311 and a ubiquitination at K317.

In some embodiments, the therapeutic agent is an antibody or antigen-binding fragment thereof that binds to a Tau peptide.

In some embodiments, the antibody is selected from the antibodies listed in Table 3.

In some embodiments, the antibody has the one or more CDR(s) comprising an amino acid sequence that is at least 80% identical to one or more CDR(s) of any one of the antibodies listed in Table 3.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A is a schematic diagrams showing an example of an experimental workflow. In a typical Tau post-translational modification profile (FLEXITau) experiment the heavy tau standard is generated in the presence of heavily labeled amino acids and added to an unlabeled endogenous sample in a ratio of approximately 1.1. After enzymatic digest and LC-MS analysis all unmodified tau peptides will be observed as pairs, featuring the light and heavy isotopologues. For modified peptides, the modification causes a mass shift, reducing the amount of detectable unmodified peptide and causing a deviation of the mixing ratio. The extent of modification on that peptide can be inferred by the amount of “missing” unmodified species.

FIG. 1B is a graph showing that peptide L/H ratios sorted from protein N- to C-terminus allows for a global visualization of modified peptides and protein regions. Blue, heavy tau; dark orange, light tau; P, phosphorylations.

FIGS. 2A-2B show the results of principal component analysis (PCA) of the Tau post-translational modification profile (FLEXITau) that parates Alzheimer patients and control subjects in cohort 1 (A), and cohort 2 (B). No significant separation is observed on the basis of sex, PMI and age using Tau modification profile data from FLEXITau across both cohorts.

FIG. 3A is a schematic overview of the study and workflow of molecular characterization of the isoform distribution and PTMs of sarkosyl soluble and insoluble Tau extracted frompost-mortem angular gyrus [BA39] brain tissues from AD and healthy age-matched individuals using mass spectrometry.

FIG. 3B is a graph showing total absolute insoluble Tau abundance quantified by FLEXITau is significantly higher in AD than in controls (t-test) whereas the soluble Tau counterpart is lower in AD than in controls (t-test).

FIG. 3C is a graph showing isoform composition of sarkosyl insoluble Tau from human post-mortem angular gyrus tissue shows that pathogenic Tau aggregates are predominately composed of 0N and 1N isoforms (yellow box plot) and 4R isoform (blue box plot) across the Tauopathy disease spectrum, whereas 1N and 4R are the predominant forms in the sarkosyl soluble fraction. Peptides were quantified using heavy-isotope labelled isoform-specific peptides and ANOVA analysis (Kruskal-Wallis) was performed using Dunn's test for multiple comparisons. Fold changes were calculated based on the mean of the concentrations measured.

FIG. 3D shows an overview of peptide coordinates of Tau including isoform-specific peptides measured in the targeted quantitative FLEXITau assays. Isoform specific regions are shown in yellow and blue. Amino acid positions for peptides are projected onto the 2N4R sequence of Tau in parentheses.

FIG. 3E show a cumulative PTM map of all mass spectrometry analyzed Tau species (soluble, insoluble, low, and high-molecular and MC1-isolated Tau) extracted from AD post-mortem brain tissue (BA39 and BA46).

FIG. 3F is a graph showing the patient frequencies of PTMs from N- to C-terminus of Tau show a wide range of frequencies from 2-90% for AD. Some high frequency sites are known AD epitopes such as the 202-205 phosphorylation site (AT8 antibody) thus pinpointing important PTM specific sites. Interestingly, some of the high frequency modifications particularly in the MBD have not been described as important to pathological Tau. Antibodies commonly used in Tau biology are annotated, antibodies used for pathology diagnosis are depicted in Brown. FLEXITau heatmaps of median peptide modification extent are overlayed with the frequency data to evaluate the stoichiometry of modifications. (****P<0.0001; ***P<0.001; **P<0.01; *P<0.1)

FIG. 4A is a graph showing the isoforms observed in the sarkosyl insoluble Tau from human post-mortem frontal cortex tissue is similar to that observed in BA39 angular gyrus tissue (see also FIG. 3B). Pathogenic Tau aggregates in AD are predominately composed of 0N and 1N isoforms and the 4R isoform in both brain regions. Peptides were quantified using heavy-isotope labelled isoform-specific peptides, and an ANOVA analysis (Kruskal-Wallis) was performed using the Dunn's test for multiple comparisons. Fold changes were calculated based on the mean concentrations measured.

FIG. 4B is a graph showing side by side analysis of FLEXITau quantified peptides listed from N—C termini in the frontal gyrus and the angular gyrus shows that the Tau modification fingerprint is similar between both regions.

FIG. 4C is a graph showing patient frequencies of PTMs from the N- to C-terminus of Tau in BA46 recapitulate the PTMs and frequencies identified in the BA39 brain region, with four PTMs only detected in BA46.

FIG. 4D is a graph showing unsupervised Euclidian hierarchical clustering analysis of Tau peptides measured using FLEXITau in the frontal gyrus (BA46) separates AD and control patients and reflects the BA39 analysis with most discriminative peptide features being the R3-R4 in the MBD and the PRR domain (see also FIG. 5A). The left panel clusters the samples and peptides using Euclidian distance, whereas the right panel shows the peptides sorted by their position within the 2N4R sequence of Tau.

FIG. 5A is a graph showing the PTM landscape of insoluble Tau is heterogenous and stratifies subjects into distinct groups reflecting disease progression. Tau PTM mapping data from shot-gun mass spectrometry from 49 AD patients and 42 age-matched healthy individuals were subjected to an unsupervised hierarchical clustering analysis using Jaccard binary clustering. The analysis separates subjects into subgroups based on unique Tau PTM signatures comprised of multiple combinations of PTMs for each subgroup. The analysis separates subjects into 4 groups a, b, c and d with c being the group with the highest Braak stage patients and a with the lowest stage Braak stages. Under the left panel we list 5 combinations of PTMs that separate the clusters into the 4 groups. The left panel ranks the PTMs according to the importance of the PTM in the hierarchical clustering, whereas the right heatmap depicts the PTMs sorted by their positions on 2N4R Tau. On the right of the figure a legend is provided for isoforms, PTM types, pathological diagnosis, clinical diagnosis and Braak stage. The type of PTM and isoform is color coded in the bar on the top of the figure and the diagnosis on the left.

FIG. 5B is a graph showing ANOVA analysis (Kruskal-Wallis), which shows that cluster b and c have higher Tau and amyloid burden than cluster a and d in the angular gyrus. This is significant for Tau across all clusters and significant for amyloid beta between cluster a and b.

FIG. 5C is a graph showing supervised PLS-DA analysis of the modified peptide intensities from MaxQuant separates the subjects according to their pathological diagnosis and identifies 25 peptides to be most discriminative modified peptides. The VIP plot is provided on the right and ranks these modifications based on their importance in separating AD from control subjects across the 2 cohorts of patients studied. The red and blue squares show if a peptide is decreased or increased in the respective disease groups.

FIG. 6A is a graph showing analysis of the posttranslational modification landscape of sarkosyl soluble tau from Alzheimer patients and matched controls. Posttranslational modifications mapped on sarkosyl soluble Tau extracted from angular gyrus (BA39) and frontal gyrus (BA46) post-mortem brain tissue shows that PTMs associated with physiological function dominate the profiles for both AD and control subjects.

FIG. 6B is a graph showing that principal component analysis does not separate the patients and control subjects based on the soluble modified tau peptides (sarkosyl soluble fraction) extracted from post-mortem angular gyrus (BA39) brain tissue.

FIG. 7A is a graph showing the FLEXITau analysis of AD patients and age-matched controls show heterogeneity in quantitative modification profiles. FLEXITau provides a measure of the extent of modification of peptides in Tau. The left panel ranks the extent of modification for each measured peptide according to the importance of the peptide in the hierarchical clustering, whereas the right heatmap depicts the peptides sorted by their positions on 2N4R Tau. On the right of the figure a legend is provided for the extent of modification of each peptide, pathological diagnosis, clinical diagnosis and Braak stage for the cohort 1 subjects. Unsupervised Euclidian hierarchical clustering of Tau peptides measured using FLEXITau in cohort 1 (29 AD vs 28 CTR). The FLEXITau clustering analysis separates the subjects into 3 major groups x, y and z with the most distinctive features being the stoichiometry of the C-terminal peptide, the R3-R4 in the MBD and the PRR domain.

FIG. 7B is a graph showing that supervised PLS-DA analysis of the FLEXITau peptide modification extent separates the subjects according to their pathological diagnosis and identifies three peptides to be most discriminative (VIP scores plot). The red and blue squares show if a peptide is decreased or increased in the respective disease groups.

FIG. 7C is a graph showing the spearman correlation analysis, which shows that the PRR and 1N/2N specific peptides are anti-correlated with an increase abundance in the MBD in both cohorts.

FIG. 7D is a graph showing correlation plots of the correlating and anti-correlating peptides in cohort 1. The FLEXITau data reveals an increase in PTM extent in the PRR and C-terminus and an enrichment of the MBD in AD insoluble aggregates and provides information of the processivity of modifications.

FIG. 7E is a graph showing receiver operating characteristic (ROC) curves visualizing the classification performances of a 10-fold cross validated stochastic gradient descent model for predicting AD and control based on FLEXITau data. The model was trained on cohort 1 and tested on cohort 2, which resulted in an AUC of 0.934 and 0.985 for AD and control, respectively.

FIG. 8A is a graph showing FLEXITau analysis cohort 2 of AD patients and age-matched control subjects. Hierarchical clustering of Tau peptides measured using FLEXITau in cohort 2. Peptides in the left heatmap are sorted by the 2N4R Tau sequence, whereas hierarchical clustering analysis was performed on the peptides of the right heatmap.

FIG. 8B is a graph showing supervised PLS-DA analysis of the FLEXITau peptide modification extent that separates the subjects according to their pathological diagnosis and identifies three peptides to be most discriminative (VIP scores plot), which are validated across the cohorts.

FIG. 8C is a graph showing spearman correlation analysis, which shows that the PRR and 1N/2N specific peptides are anti correlated with an increase abundance in the MBD in cohort 2.

FIG. 9A is a schematic illustration of the workflow used to isolate soluble fractions of size separated seeding-competent High Molecular Weight Tau oligomers and seeding incompetent Low Molecular Weight Tau (monomeric Tau).

FIG. 9B is a graph showing comparative analysis of FLEXITau quantified peptides listed from N—C termini in the different fractions.

FIG. 9C is a graph showing the heatmap that displays median peptide modification extent calculated from quantitative FLEXITau assays obtained by targeted mass spectrometry experiments

FIG. 9D is a graph showing cumulative maps of PTM maps of seeding competent and incompetent Tau separated by size are shown including: monomeric Tau-LMW; sarkosyl soluble Tau (monomeric); oligomeric Tau-HMW; MC1-isolated Tau; and sarkosyl insoluble fraction containing fibrillar Tau. HMW and LMW Tau species were isolated by size-exclusion chromatography from whole brain of 4 control subjects and 4 AD patients. Monomeric and LMW oligomers (44-150 kDa) of Tau exhibits 9 modifications, oligomeric Tau from the HMW oligomers exhibit 26 modifications and fibrillar Tau from the sarkosyl insoluble fraction of 33 AD patients cumulatively display over 84 modifications.

FIG. 9E is a schematic illustration showing the size differences of the size exclusion, antibody and detergent fractionated forms of Tau.

FIG. 10A is a graph showing charge distribution of 2N4R Tau. The graph shows that the PTMs in the late stage of the disease neutralize the positive charges in the MBD and add negative charge in the PRR region.

FIG. 10B is a graph showing that prediction of natural disordered regions demonstrates that MBD domain becomes increasingly disordered by starting at position 305-330 which becomes heavily ubiquitinated in the late stage of Alzheimer's disease.

FIG. 11A is a schematic illustration of sequential accumulation of different PTMs at different stages of disease. The increased phosphorylation in the PRR is followed by acetylation and ubiquitination in the MBD as the disease progresses.

FIG. 11B is a schematic illustration that summarizes the FLEXITau data showing a three-step process that lead to Tau aggregation across patients. The 1N and 2N isoforms are underrepresented in all insoluble Tau. An early event that is observed is the cleavage of the C-terminal and this is followed by the phosphorylation of the PRR and the enrichment of the MBD. The enrichment of MBD is notable in AD patients compared to control.

FIG. 11C is a schematic illustration of a model for Tau fibril formation based on the posited stoichiometric PTM analysis. The 0N and 4R isoforms are predisposed to aggregation. A cascade of PTMs including C-terminal cleavage, negatively charged phosphorylation in the PRR, followed by charge neutralizing acetylation and ubiquitination in the enriched MBR are progressive steps in the process of Tau fibril formation and AD disease progression.

FIG. 12 is a schematic illustration of epitopes of some antibodies that bind to Tau.

FIG. 13A is a schematic illustration of immunoprecipitation/MS workflow.

FIG. 13B depicts PCA of modified peptide intensities in the sarkosyl insoluble fraction separates AD (n=46) from CTRL (n=40) cases.

FIG. 13C is a loading plot showing which modified peptides contribute to the loading of the principal components shown in FIG. 13B.

FIG. 13D depicts a list of modifications represented by the top 10 contributing peptides in FIG. 13C and names of available commercial antibodies.

FIG. 13E depicts a map of epitope specificities of selected commercial antibodies.

FIG. 13F is a representative blot and quantification of immunoreactivity with pan-Tau antibody S262 for immunodepleted lysate.

FIG. 13G is a representative blot and quantification of immunoreactivity with pan-Tau antibody S262 for IP eluate.

FIG. 14 shows identified posttranslational modifications and extent of modification of HMW and LMW Tau prepared by size exclusion chromatography. LMW (top) and HMW (bottom) Tau fractions prepared from 4 AD cases were analyzed by label-free mass spectrometry to identify PTMs and by FLEXITau to measure extent of modification (bar overlay). The targets ubK311/ubK317 and pT231/pS235 were observed in HMW Tau. Additional targets were prioritized bases on a) specificity to HMW vs LMW Tau b) extent of modification at PTM site.

FIGS. 15A-15D show IP of Tau using commercially available Tau antibodies-seeding assays. FIG. 15A shows YFP puncta counts in HEK293RD-P301S cells 48 hours after transfection with IP eluates (FIG. 15A) or immunodepleted lysates of IPs with commercial Tau antibodies (FIG. 15C). FIGS. 15B and 15D show Puncta counts from FIGS. 15A and 15C respectively normalized to the amount of Tau in each preparation as measured by immunoblot with pan-Tau antibody. This normalization was performed to give a measure of specificity. Note that while AT180 and FK2 did not immunodeplete significant amounts of seeding Tau species, the IP eluates were able to seed aggregation.

FIG. 15E shows representative fluorescence microscopy images of cells 48 hours after transfection with eluates from Tau IPs.

FIG. 16A shows relative intensities of detected modified peptides in each of the IP elutes. Peptides intensities were normalized to total Tau intensity to give a relative occupancy measure, and then scaled for each modified peptide.

FIG. 16B shows that the modified peptide data in FIG. 16A was subjected to unsupervised hierarchical clustering, dividing the data in 4 clusters, containing AT8, PHF1, AT180, and FK2. 2) RN235. 3) AT270 and 4) MIgG, Tau7, HT7, Tau 7, Tau12, AT100, and Tau5.

FIG. 16C shows the correlations between modified peptides in IP eluates.

FIG. 16D shows the correlations between seeding of eluates in biosensor seeding assay and modified peptide intensities.

FIG. 17 shows the enriched proteome for Tau antibodies IPs. Relative of abundance of proteins in eluate of immunoprecipitation for each of the antibodies compared to control. Tau was enriched in all the Ips except AT100 and RN235. Ubiquitin was enriched in all Ips except AT100, RN235, AT8, and Tau7, but with different stoichiometries. FK2, AT180, and PHF1 had the highest ubiquitin to Tau ratio.

### DETAILED DESCRIPTION

This disclosure provides, in part, methods for diagnosing a subject for having a tauopathy, e.g., an Alzheimer's disease (AD), methods for treating a subject having a tauopathy, e.g., Alzheimer's disease, and methods for detecting post-translational modifications (PTMs) associated with a tauopathy, e.g., an Alzheimer's disease, in a tau protein.

Post-translation modifications (PTMs) associated with tau protein in tauopathies, e.g., Alzheimer's disease, are identified. A targeted, high-throughput, quantitative mass spectrometry (MS) method called FLEXITau (Full-Length Expressed Stable Isotope-labeled Tau) was previously described which provides absolute quantification and unbiased stoichiometric information of Tau modifications from N- to the C-terminus (see, e.g., Mair et al., 2016; and PCT Application No. PCT/US2016/053357, the entire contents of the foregoing are incorporated herein by reference). The present disclosure further determines the absolute quantities (molar concentration) of pathologic Tau in AD; provides a comprehensive map of PTMs in human AD; determines the distribution of isoforms of Tau in the pathologic and non-pathologic forms of Tau; shows the extent (stoichiometry) of modification; and the heterogeneity of modification profiles across all AD patients and control subjects.

To understand the importance of PTMs in disease a comprehensive analysis of PTMs in a cohort requires numerous levels of information: including a) Type of PTM b) PTM localization c) PTM frequency in the subject cohort d) PTM stoichiometry. Thus, this disclosure provides a quantitative and qualitative protein profiling of human tissues. FLEXITau and unbiased mass spectrometry-based proteomics is used to characterize and map the PTM landscape of pathological Tau for AD. Supervised and unsupervised data analyses were used to determine the most relevant molecular features of Tau pathology (Fitzpatrick et al., 2017) that are important in the aggregation of Tau. Furthermore, size-resolved fractions of Tau (high and low molecular weight Tau and MC1-isolated Tau) were studied to identify the minimal set of PTMs associated with seeding potential and the role of PTMs in the ontogeny of fibril formation. These qualitative and quantitative data provide key insights into the role of PTMs in the progression of disease and identification of key targets for the development of therapeutic antibodies, imaging reagents and diagnostics for AD.

### Tauopathies

Human Tau is encoded on chromosome 17q21 (see, e.g., Neve R L et al., Brain Res. 1986 December; 387(3):271-80). The protein occurs mainly in the axons of the central nerve system (CNS) and consists largely of six isoforms generated by alternative splicing (see, e.g., Goedert M et al., EMBO J. 1989 February; 8(2):393-9). They differ by the presence or absence of two near-amino-terminal inserts of 29 residues each, encoded by exons 2 and 3, and by one of the repeats (R2, 31 residues) in the carboxy-terminal half A representative sequence of human tau 2N4R isoform is shown below:

Amino Acid Sequence of 2N4R Tau Protein
>sp|P10636-8|TAU_HUMAN Isoform Tau-F of Microtubule-associated protein tau
OS = Homo sapiens OX = 9606 GN = MAPT
SEQ ID NO: 1
MAEPRQEFEVMEDHAGTYGLGDRKDQGGYTMHQDQEGDTDAGLKESPLQTPTEDGSEEPG
SETSDAKSTPTAEDVTAPLVDEGAPGKQAAAQPHTEIPEGTTAEEAGIGDTPSLEDEAAG
HVTQARMVSKSKDGTGSDDKKAKGADGKTKIATPRGAAPPGQKGQANATRIPAKTPPAPK
TPPSSGEPPKSGDRSGYSSPGSPGTPGSRSRTPSLPTPPTREPKKVAVVRTPPKSPSSAK
SRLQTAPVPMPDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRVQSKIGSLDNI
THVPGGGNKKIETHKLTFRENAKAKTDHGAEIVYKSPVVSGDTSPRHLSNVSSTGSIDMV
DSPQLATLADEVSASLAKQGL

Tauopathies represent a large group of proteopathies featuring aggregates of an altered form of the microtubule associated protein tau. The term “tauopathy” refers to tau-related disorders or conditions, e.g., Alzheimer's Disease (AD), Progressive Supranuclear Palsy (PSP), Corticobasal Degeneration (CBD), Pick's Disease (PiD), Argyrophilic grain disease (AGD), Frontotemporal dementia and Parkinsonism associated with chromosome 17 (FTDP-17), Parkinson's disease, stroke, traumatic brain injury, mild cognitive impairment and the like.

Alzheimer's disease (AD) is a kind of tauopathy. It is a chronic neurodegenerative disease. The most common early symptom is difficulty in remembering recent events (short-term memory loss). As the disease advances, symptoms can include problems with language, disorientation (including easily getting lost), mood swings, loss of motivation, not managing self-care, and behavioral issues.

Braak staging is often used to classify the degree of pathology in Alzheimer's disease. The first two stages are characterized by an either mild or severe alteration of the transentorhinal layer Pre-alpha (transentorhinal stages I-II). The two forms of limbic stages (stages III-IV) are marked by a conspicuous affection of layer Pre-alpha in both transentorhinal region and proper entorhinal cortex. In addition, there is mild involvement of the first Ammon's horn sector. The hallmark of the two isocortical stages (stages V-VI) is the destruction of virtually all isocortical association areas. A detailed description of Braak staging can be found e.g., in Braak et al., “Neuropathological staging of Alzheimer-related changes.” Acta neuropathologica 82.4 (1991): 239-259, which is incorporated herein by reference in its entirety.

The pathway leading from soluble and monomeric to hyperphosphorylated, insoluble and filamentous tau protein is at the center of tauopathies. Usually, the first tau aggregates form in a few nerve cells in discrete brain areas. These become self propagating and spread to distant brain regions in a prion-like manner. In a clinical setting, the clinical syndromic diagnosis is often determined by the patient's symptoms and deficits, while the pathological diagnosis is defined by characteristic types and distribution of the tau inclusions and of neuron loss.

Recent studies using cellular and animal models have suggested that tau pathology progresses by trans-cellular propagation. The process of propagation is mediated by certain species of extracellular tau, which are taken up by recipient cells and serve as a seed for tau aggregation. This activity of the tau peptides is called the seeding activity. Tau propagation can lead to dementia. Multiple forms of tau with different molecular weights derived from recombinant tau or brain lysates exert seeding activity.

### Subjects

The terms “subject” and “patient” are used interchangeably throughout the specification and describe an animal, human or non-human, to whom treatment according to the methods of the present invention is provided. Human patients can be adult humans or juvenile humans. In some embodiments, humans can have an age of above 10, 20, 30, 35, 40, 45, 50, 55, 60, 65, 70, 75, 80, 85, or 90 years old. In some embodiments, the subject is a mammal. In some embodiments, the term “subject”, as used herein, refers to a human (e.g., a man, a woman, or a child).

The subject can be symptomatic (e.g., the subject presents symptoms associated with tauopathies (e.g., AD, AGD, CBD, PiD, PSP), such as, for example changes in personality, behavior, sleep patterns, and executive function, memory loss, confusion, inability to learn new things, difficulty carrying out multistep tasks, problems coping with new situations, hallucinations, delusions, and paranoia, impulsive behavior, inability to communicate, weight loss, seizures, skin infections, difficulty swallowing, groaning, moaning, grunting, increased sleeping, lack of control of bowel and bladder, disorders of word finding, disorders of reading and writing, disorientation, supranuclear palsy, a wide-eyed appearance, difficulty in swallowing, unwarranted anxiety, irrational fears, oniomania, impaired regulation of social conduct (e.g., breaches of etiquette, vulgar language, tactlessness, disinhibition, misperception), passivity, low motivation (aboulia), inertia, over-activity, pacing and wandering, etc. The subject can be asymptomatic (e.g., the subject does not present symptoms associated with a tauopathy, or the symptoms have not been recognized).

In addition to humans, subjects include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

### Sample Collection and Preparation

Samples for use in the methods described herein include various types of samples from a subject.

In some embodiments, the sample is a “biologic sample”. As used herein, the term “biological sample” or “sample” refers to a sample obtained or derived from a subject. By way of example, the sample may be selected from the group consisting of body fluids, blood, whole blood, plasma, serum, mucus secretions, urine or saliva. In some embodiments the sample is, or comprises a blood sample. The preferred biological source for detection of the biomarkers is a blood sample, a serum sample or a plasma sample. In some embodiments, the sample is cerebrospinal fluid (CSF) or a brain tissue.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as a blood, e.g., blood that was previously isolated from a patient. Thus, obtain is used to mean collection and/or removal of the sample from the subject. Furthermore, “obtain” is also used to mean where one receives the sample from another who was in possession of the sample previously.

In some embodiments, a reference sample is obtained from at least one individual not suffering from a tauopathy. In some other embodiments, the reference sample is obtained from at least one individual previously diagnosed as having a tauopathy (e.g., AD, AGD, CBD, PiD, PSP). In some embodiments, the reference sample comprises a predetermined, statistically significant reference analyte levels.

In some embodiments, the sample is collected from the brain of a subject, e.g., brain tissue. In some embodiments, the sample is collected from cerebrospinal fluid or plasma.

In some embodiments, the sample is collected from a biopsy. A biopsy is a sample of tissue taken from the body of a living subject. A biopsy sometimes also refers to the medical procedure that removes tissue from a living subject. In some embodiments, the sample can be collected through a punch biopsy. A punch biopsy is done with a circular blade ranging in size from 1 mm to 8 mm. In some embodiments, the sample can be collected from fine-needle aspiration biopsy (FNAB or FNA). Fine-needle aspiration biopsy is a procedure used to investigate superficial (just under the skin) lumps or masses. In some embodiments, a thin, hollow needle is inserted into the body to collect samples.

In some embodiments, the sample is from a live subject. For example, the sample can be collected from a subject during a medical procedure, e.g., a surgery.

In some embodiments, samples are collected from post-mortem specimens, e.g., human post-mortem brain specimens.

In some embodiments, brain tissue can be obtained from Brodmann area 39 (BA39) angular gyrus brain blocks.

In some embodiments, biopsy samples are homogenized and clarified by centrifugation. Supernatants containing tau proteins are pooled and used as a crude tau fraction (unfractionated homogenate).

In some embodiments, samples are collected from cultured cells, e.g., from E. coli or sf9 cells. In some embodiments, samples are collected from the brain tissue of model animals.

### Post Translational Modifications (PTMs)

Post-translational modification (PTM) refers to the covalent and generally enzymatic modification of proteins following protein biosynthesis. Proteins are synthesized by ribosomes translating mRNA into polypeptide chains, which may then undergo PTM to form the mature protein product. PTMs are important components in cell signaling, as for example when prohormones are converted to hormones.

Post-translational modifications can occur on the amino acid side chains or at the protein's C- or N-termini (see, e.g., Pratt, Donald Voet et al., (2006). Fundamentals of biochemistry: life at the molecular level (2. ed.), ISBN 978-0-471-21495-3). They can extend the chemical repertoire of the 20 standard amino acids by modifying an existing functional group or introducing a new one such as phosphate. Phosphorylation is a very common mechanism for regulating the activity of enzymes and is the most common post-translational modification (see, e.g., Khoury G A et al., Scientific Reports, 1: 90). Many eukaryotic and prokaryotic proteins also have carbohydrate molecules attached to them in a process called glycosylation, which can promote protein folding and improve stability as well as serving regulatory functions. Attachment of lipid molecules, known as lipidation, often targets a protein or part of a protein attached to the cell membrane.

The post-translational modifications (PTMs) identified in the methods described herein can be any type of PTMs. In some embodiments, the PTMs are one or more of phosphorylation, glycosylation, glycation, prolyl-isomerization, cleavage or truncation, nitration, polyamination, ubiquitination, acetylation, methylation, dimethylation, trimethylation or sumoylation. Unless otherwise indicated, all numbering of amino acid residues of tau protein described herein is based on the human 2N4R isoform.

### Phosphorylation

Phosphorylation is a significant post-translational modification (PTM) affecting a protein's shape, behavior within the cell, and function. Kinase and phosphatase enzymes, respectively, remove or add phosphate groups, mostly to serine, threonine, and tyrosine residues. These amino acids all contain a nucleophilic group that reacts with adenosine triphosphate (ATP), replacing an oxygen on the terminal phosphorous and ejecting adenosine diphosphate (ADP).

While kinases and phosphatases function in all living cells they are particularly active in eukaryotes, in which phosphorylation is one of the most significant post-translational modifications. Approximately 30% of proteins in eukaryotic cells are subject to phosphorylation, which implies the existence of a huge, nearly untapped avenue for pharmacologic intervention in nearly all serious human diseases.

Determining the extent and locations of phosphorylation is critical to understanding biochemical pathways in cells, particularly how cellular activity is activated or suppressed. Through phosphorylation, cells regulate growth, apoptosis, cell cycle progression, and signal transduction. As such the ubiquitous, constant addition and removal of phosphate from protein serves as a specific on/off switch for individual cell operations, and for the cell as a whole.

In some embodiments, the one or more post translational modifications (PTMs) identified using the methods described herein include at least one phosphorylation. In some embodiments, the phosphorylation(s) are at one or more of positions Y29, T30, T39, S46, S56, S68, T69, T71, T102, T111, S113, T153, T175, T181, S184, S185, S191, S198, S199, S202, T205, S210, T212, S214, T217, T220, T231, S235, S237, S238, S241, S258, S262, T263, S289, S293, S305, Y310, S316, S352, S356, T361, T386, Y394, S396, S400, T403, S404, S409, S412, S413, T414, S422, S433, and S435 in a tau protein. In some embodiments, the phosphorylation(s) are at one or more of positions S198, S199, S202, and T205 in a tau protein. In some embodiments, the phosphorylation(s) are at one or more of positions S212, S214, and S217 in a tau protein. In some embodiments, the phosphorylation(s) are at one or more of positions T181 and T231 in a tau protein. In some embodiments, the phosphorylation(s) are at one or more of positions S198, S199, S202, and T205 in a tau protein. Numbering is based on the 2N4R isoform of tau protein (SEQ ID NO: 1).

In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more amino acid residues are phosphorylated in a tau protein from a biological sample. In some embodiments, the biological sample is obtained from an Alzheimer's disease patient.

### Acetylation

Acetylation (or in IUPAC nomenclature ethanoylation) describes a reaction that introduces an acetyl functional group into a chemical compound. Deacetylation is the removal of an acetyl group.

Acetylation refers to the process of introducing an acetyl group (resulting in an acetoxy group) into a compound, namely the substitution of an acetyl group for an active hydrogen atom. A reaction involving the replacement of the hydrogen atom of a hydroxyl group with an acetyl group (CH₃CO) yields a specific ester, the acetate. Acetic anhydride is commonly used as an acetylating agent reacting with free hydroxyl groups.

Acetylation is an important modification of proteins in cell biology; and proteomics studies have identified thousands of acetylated mammalian proteins (see, e.g., Choudhary et al., Science. 325 (5942): 834-840; and Fritz et al., J Proteome Res. 11 (3): 1633-1643). Acetylation occurs as a co-translational and post-translational modification of proteins, for example, histones, p53, and tubulins. Among these proteins, chromatin proteins and metabolic enzymes are highly represented, indicating that acetylation has a considerable impact on gene expression and metabolism.

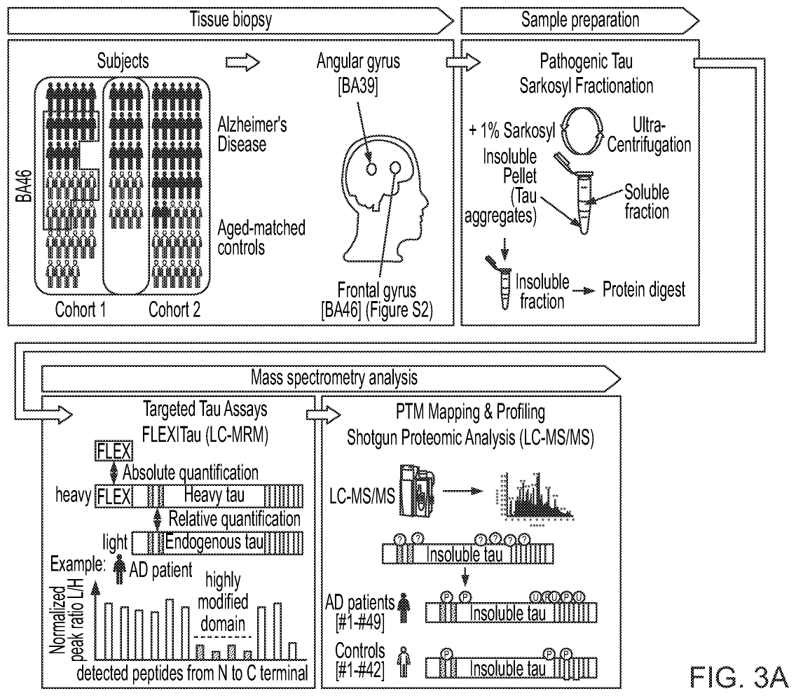

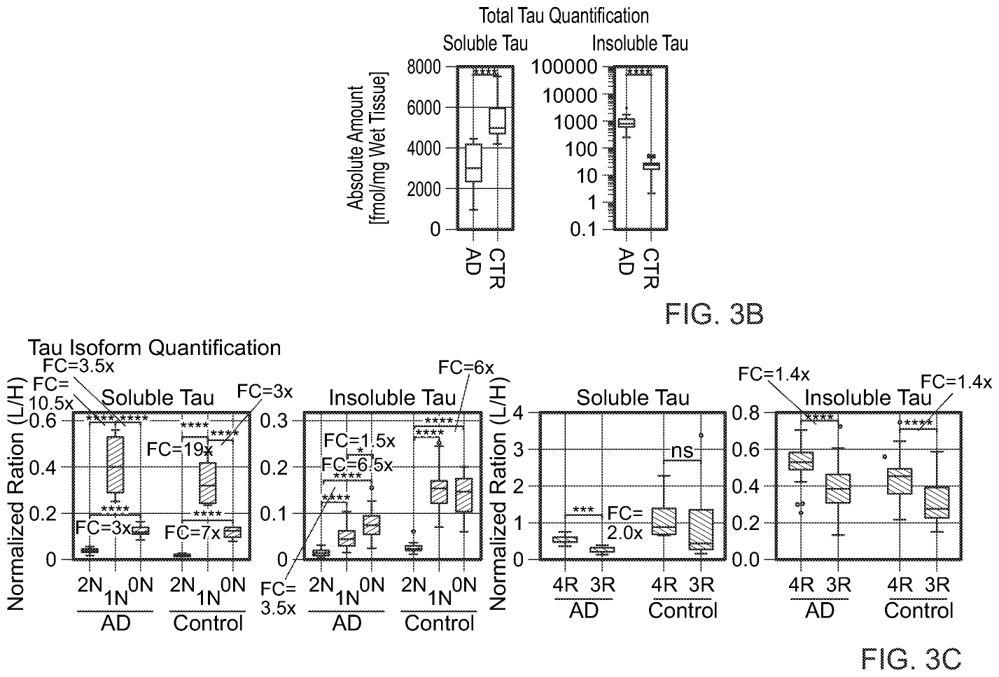

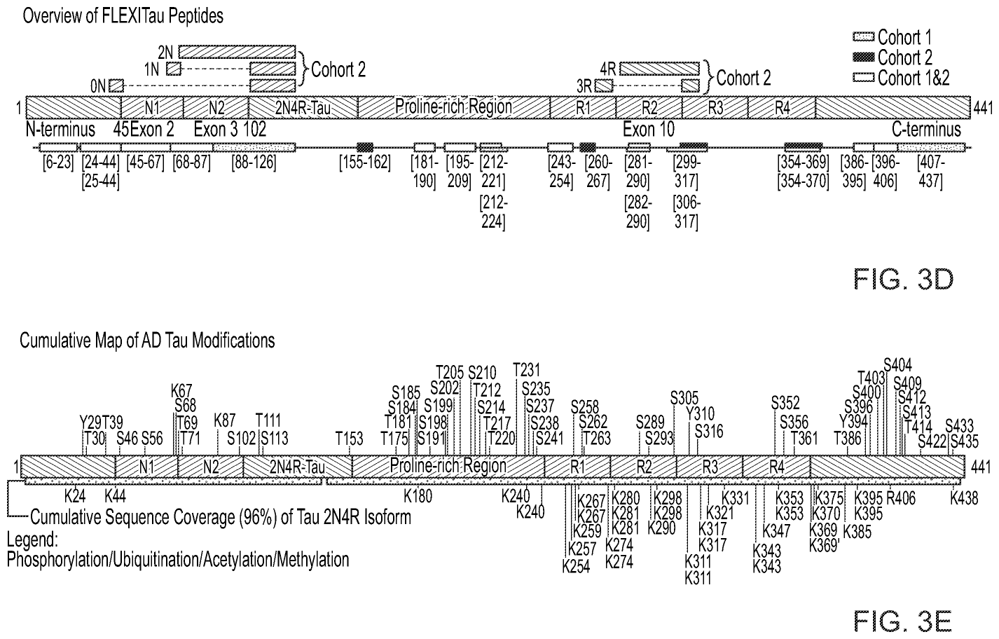

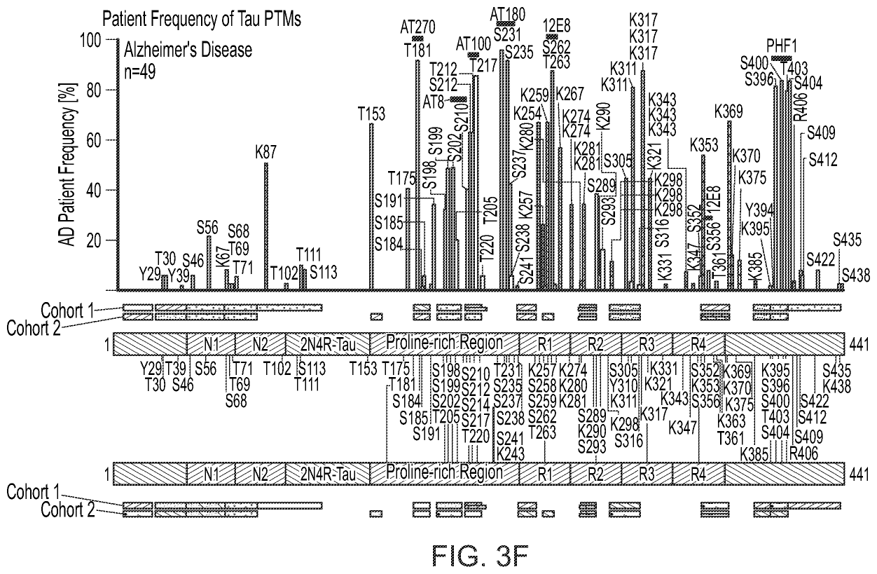

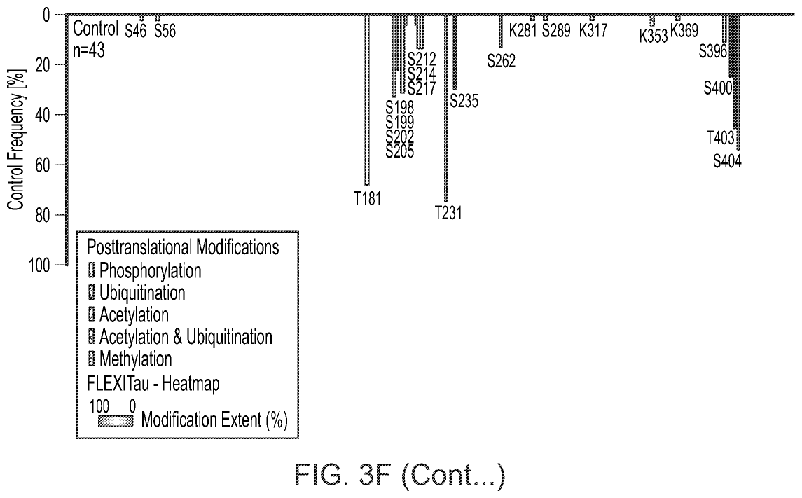

In some embodiments, the one or more post translational modifications (PTMs) identified using the methods described herein include at least one acetylation. In some embodiments, the acetylation(s) are at one or more of positions K24, K44, K240, K267, K274, K280, K281, K298, K311, K317, K331, K343, K347, K353, K369, K370, K375, K385, and K395 in a tau protein (FIG. 3E). In some embodiments, the acetylation(s) are at one or more of positions K353, K369, K370, and K375 in a tau protein. Numbering is based on the 2N4R isoform of tau protein (SEQ ID NO: 1).

In some embodiments, at least 1, 2, 3, 4, 5, 6, 7, 8, 9, 10, 11, 12, 13, 14, 15, 16, 17, 18, 19 or more amino acid residues are acetylated in a tau protein from a biological sample. In some embodiments, the biological sample is obtained from an Alzheimer's disease patient.

### Ubiquitination

Ubiquitination is the addition of ubiquitin molecules to lysine residues of a protein. Following ubiquitination, most proteins are targeted to the 26S proteosome for degradation. The ubiquitination system involves numerous proteins, but the specificity of the system depends on the specific E3 ubiquitin ligase enzyme employed, which attaches an ubiquitin molecule to the correct substrate.

In some embodiments, the one or more post translational modifications (PTMs) identified using the methods described herein include at least one ubiquitination. In some embodiments, the ubiquitination(s) are at one or more of positions K180, K240, K254, K257, K259, K267, K274, K281, K290, K298, K311, K317, K321, K343, K353, K369, and K395 in a tau protein (FIG. 3E). Numbering is based on the 2N4R isoform of tau protein (SEQ ID NO: 1).

In some embodiments, at least 1, 2, 3, 4, 5, 6, 7, 8, 9, 10, 11, 12, 13, 14, 15, 16, 17 or more amino acid residues are ubiquitinated in a tau protein from a biological sample. In some embodiments, the biological sample is obtained from an Alzheimer's disease patient.

### Methylation

Gene expression is controlled in eukaryotes in various ways, including through methylation of DNA. DNA methylation is a biological process by which methyl groups are added to the DNA molecule, thereby changing gene activity without changing the underlying DNA sequence. In mammals, epigenetic modifications such as DNA methylation occur at cytosine/guanine (CG) dinucleotides. DNA methylation is typically found in promoter regions (known as CpG islands) and are associated with transcriptional repression. For example, a gene can be activated (e.g., “turned on”) in the presence of open chromatin and acetylated histones. In this instance, nucleotides generally remain unmethylated. However, in the presence of a methylated nucleotide (e.g., a methylated cytosine), a chromosome can be condensed, resulting in de-activation of gene expression (e.g., expression is “turned off”). Thus, when located in a gene promoter, DNA methylation typically acts to repress gene transcription. Two DNA bases, cytosine and adenine, can be methylated. Cytosine methylation is widespread in both eukaryotes and prokaryotes. Methylation of cytosine to form 5-methylcytosine occurs at the same 5′ position on the pyrimidine ring where the DNA base thymine's methyl group is located; the same position distinguishes thymine from the analogous RNA base uracil, which has no methyl group. Spontaneous deamination of 5-methylcytosine converts it to thymine. This results in a T:G mismatch that can be identified through sequencing techniques.

In recent decades, DNA methylation has been studied extensively, including how it occurs and where it occurs, and it has been discovered that methylation is an important component in numerous cellular processes, including embryonic development, genomic imprinting, X-chromosome inactivation, and preservation of chromosome stability. DNA methylation is used as a differentiating marker in various settings, including cancer, neurology, some genetic diseases, development, cellular differentiation, model organism understanding, and during therapy (e.g., drug treatment).

In some embodiments, the one or more post translational modifications (PTMs) identified using the methods described herein include at least one methylation. In some embodiments, the methylation(s) are at one or more of positions K67, K87, R406, and K438 in a tau protein (FIG. 3E). Numbering is based on the 2N4R isoform of tau protein (SEQ ID NO: 1).

In some embodiments, at least 1, 2, 3, 4 or more amino acid residues are methylated in a tau protein from a biological sample. In some embodiments, the biological sample is obtained from an Alzheimer's disease patient.

Table 1 lists the amino acid positions where PTMs can be detected in a tauopathy, e.g., Alzheimer's disease.

TABLE 1
PTMs in Tau protein

| Position on 2N4R Tau | |
|---|---|
| Protein | Modification Type |
| K24 | Acetylation |
| Y29 | Phosphorylation |
| T30 | Phosphorylation |
| T39 | Phosphorylation |
| K44 | Acetylation |
| S46 | Phosphorylation |
| S56 | Phosphorylation |
| S61 | Phosphorylation |
| S64 | Phosphorylation |
| K67 | Methylation |
| S68 | Phosphorylation |
| T69 | Phosphorylation |
| T71 | Phosphorylation |
| K87 | Methylation |
| T102 | Phosphorylation |
| T111 | Phosphorylation |
| S113 | Phosphorylation |
| T153 | Phosphorylation |
| T175 | Phosphorylation |
| K180 | Ubiquitination |
| T181 | Phosphorylation |
| S184 | Phosphorylation |
| S185 | Phosphorylation |
| S191 | Phosphorylation |
| S198 | Phosphorylation |
| S199 | Phosphorylation |
| S202 | Phosphorylation |
| T205 | Phosphorylation |
| S210 | Phosphorylation |
| T212 | Phosphorylation |
| S214 | Phosphorylation |
| T217 | Phosphorylation |
| T220 | Phosphorylation |
| T231 | Phosphorylation |
| S235 | Phosphorylation |
| S237 | Phosphorylation |
| S238 | Phosphorylation |
| K240 | Acetylation; Ubiquitination |
| S241 | Phosphorylation |
| K254 | Ubiquitination |
| K257 | Ubiquitination |
| S258 | Phosphorylation |
| K259 | Ubiquitination |
| S262 | Phosphorylation |
| T263 | Phosphorylation |
| K267 | Acetylation; Ubiquitination |
| K274 | Acetylation; Ubiquitination |
| K280 | Acetylation |
| K281 | Acetylation; Ubiquitination |
| S289 | Phosphorylation |
| K290 | Ubiquitination |
| S293 | Phosphorylation |
| K298 | Acetylation; Ubiquitination |
| S305 | Phosphorylation |
| Y310 | Phosphorylation |
| K311 | Acetylation; Ubiquitination |
| S316 | Phosphorylation |
| K317 | Acetylation; Ubiquitination |
| K321 | Ubiquitination |
| K331 | Acetylation |
| K343 | Acetylation; Ubiquitination |
| K347 | Acetylation |
| S352 | Phosphorylation |
| K353 | Acetylation; Ubiquitination |
| S356 | Phosphorylation |
| T361 | Phosphorylation |
| K369 | Acetylation; Ubiquitination |
| K370 | Acetylation |
| K375 | Acetylation |
| K385 | Acetylation |
| T386 | Phosphorylation |
| Y394 | Phosphorylation |
| K395 | Acetylation; Ubiquitination |
| S396 | Phosphorylation |
| S400 | Phosphorylation |
| T403 | Phosphorylation |
| S404 | Phosphorylation |
| R406 | Methylation |
| S409 | Phosphorylation |
| S412 | Phosphorylation |
| S413 | Phosphorylation |
| T414 | Phosphorylation |
| S416 | Phosphorylation |
| S422 | Phosphorylation |
| S433 | Phosphorylation |
| S435 | Phosphorylation |
| K436 | Phosphorylation |
| K438 | Methylation |

Techniques for detecting and quantifying PTMs include, but are not limited to, kinase activity assays, phospho-specific antibody assays, Western blot, enzyme-linked immunosorbent assays (ELISA), cell-based ELISA, intracellular flow cytometry, mass spectrometry, multi-analyte profiling, methylation-sensitive restriction enzyme digestion, bisulfite treatment and sequencing, and deamination and sequencing.

In some embodiments, one or more tau peptide fragments of a tau protein in a biological sample are identified in the methods described herein. In some embodiments, the one or more tau peptide fragments comprises different amount of PTMs compared to a reference sample. Table 2 lists some example tau peptide fragments that are detected using the methods described herein.

TABLE 2
Fragments of Tau Protein having PTMs

| Amino Acid | | |
|---|---|---|
| Positions in Tau | | |
| (numbering of | | |
| SEQ ID NOs: 2-22 | | |
| is based on | | SEQ |
| SEQ ID NO: 1) | Tau Peptide Sequence | ID NO |
| [6-23] | QEFEVMEDHAGTYGLGDR | 2 |
| [24-44] | KDQGGYTMHQDQEGDTDAGLK | 3 |
| [25-44] | DOGGYTM[Oxi]HQDQEGDTDAGLK | 4 |
| [45-67] | ESPLQTPTEDGSEEPGSETSDAK | 5 |
| [68-87] | STPTAEDVTAPLVDEGAPGK | 6 |
| [88-126] | QAAAQPHTEIPEGTTAEEAGIGDTPSLEDEAAGHVTQAR | 7 |
| [155-162] | GAAPPGQK | 8 |
| [181-190] | TPPSSGEPPK | 9 |
| [195-209] | SGYSSPGSPGTPGSR | 10 |
| [212-221] | TPSLPTPPTR | 11 |
| [212-224] | TPSLPTPPTREPK | 12 |
| [243-254] | LQTAPVPM[Oxi]PDLK | 13 |
| [260-267] | IGSTENLK | 14 |
| [281-290] | KLDLSNVQSK | 15 |
| [282-290] | LDLSNVQSK | 16 |
| [299-317] | HVPGGGSVQIVYKPVDLSK | 17 |
| [354-369] | IGSLDNITHVPGGGNK | 18 |
| [354-370] | IGSLDNITHVPGGGNKK | 19 |
| [386-395] | TDHGAEIVYK | 20 |
| [396-406] | SPVVSGDTSPR | 21 |
| [407-437] | HLSNVSSTGSIDM[Oxi]VDSPQLATLADEVSASLAK | 22 |
| 0N | AEEAGIGDTPSLEDEAAGHVTQAR | 23 |
| 1N | STPTAEAEEAGIGDTPSLEDEAAGHVTQAR | 24 |
| 3R | VQIVYKPVDLSK | 25 |

In some embodiments, additional fragments of tau protein are identified in the methods described herein. In some embodiments, the additional tau fragments have one or more PTMs. In some embodiments, the additional tau fragments do not have PTMs and are identified as reference fragments.

### Full-Length Expressed Stable Isotope-Labeled Tau (FLEXITau)

In some aspects, the disclosure provides methods for determining post-translational modifications and/or quantifying the amount of post-translational modifications disclosed herein utilizes a stable isotope-labeled (‘heavy’) full-length tau protein standard that is added to a biological specimen prior to sample processing and MS analysis, which is referred to herein as “FLEXITau”.

The heavy tau standard can generated by various means. In some embodiments, the longest tau isoform (4R2N, also referred to as 2N4R) is cloned into the various vectors, e.g., FLEX-vector, introducing an N-terminal artificial tag to the protein that is later used for standard purification as well as for absolute quantification of the endogenous tau. The FLEX-vector is described in, e.g., Singh, Sasha, et al. “FLEXIQuant: a novel tool for the absolute quantification of proteins, and the simultaneous identification and quantification of potentially modified peptides.” Journal of proteome research 8.5 (2009): 2201-2210, which is incorporated by reference in its entirety.

In some embodiments, heavy tau protein can be expressed in a cell free expression system in the presence of isotopically labeled aspartic acid, lysine and arginine. The triple labeling strategy can minimize co-expressed light tau standard that could lead to a bias in quantification of endogenous tau.

The tau standard is purified and is added to unlabeled endogenous sample (‘light’) in a predetermined ratio. Various ratios can be used, e.g., approx. 4:1, 3:1, 2:1, 1:1, 1:2, 1:3 or 1:4. The protein mix is subjected to enzymatic digest and LC-MS analysis. Notably, due to the mixing of light and heavy species early in the sample processing, quantification errors that might arise due to sample loss and technical variability of sample preparation are minimized. The protein mix can be digested by various enzymes, e.g., trypsin, Lys-C, Arg-C, Asp-N, Glu-C, Lys-N, thermolysin, elastase, and chymotrypsin. In some embodiments, the labeled sample and the unlabeled sample is subjected to enzymatic digest separately before they are mixed together.

All unmodified tau peptides will be present as pairs, featuring the light (unlabeled) and the heavy (labeled) isotopologue. While each pair of peptide species has varying signal intensities, the light-to-heavy (L/H) intensity ratio of all unmodified peptides reflects the initial mixing ratio (e.g., L/H=1). The phosphorylation causes a mass shift, reducing the amount of detectable unmodified peptide. In consequence, a deviation of the mixing ratio is observed. The extent of modification on that peptide can be inferred by the amount of ‘missing’ unmodified species.

Plotting the L/H ratio of all peptides sorted from N- to C terminal results in an intuitive representation of the PTM landscape across tau protein, where individual modifications as well as modified peptide regions can be quantitatively inferred.

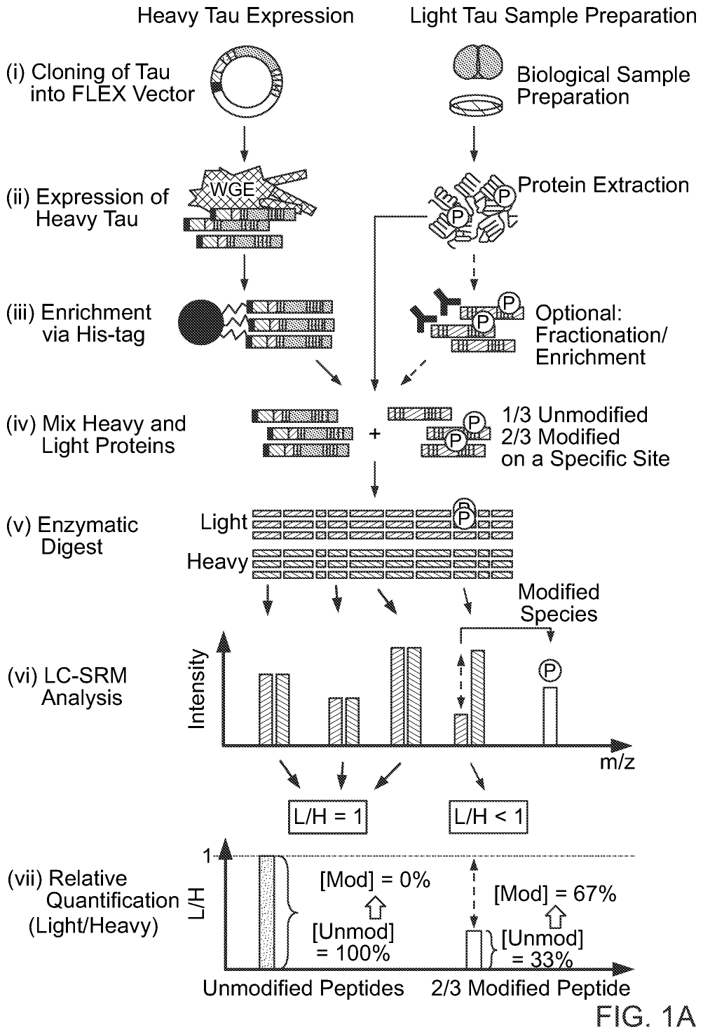

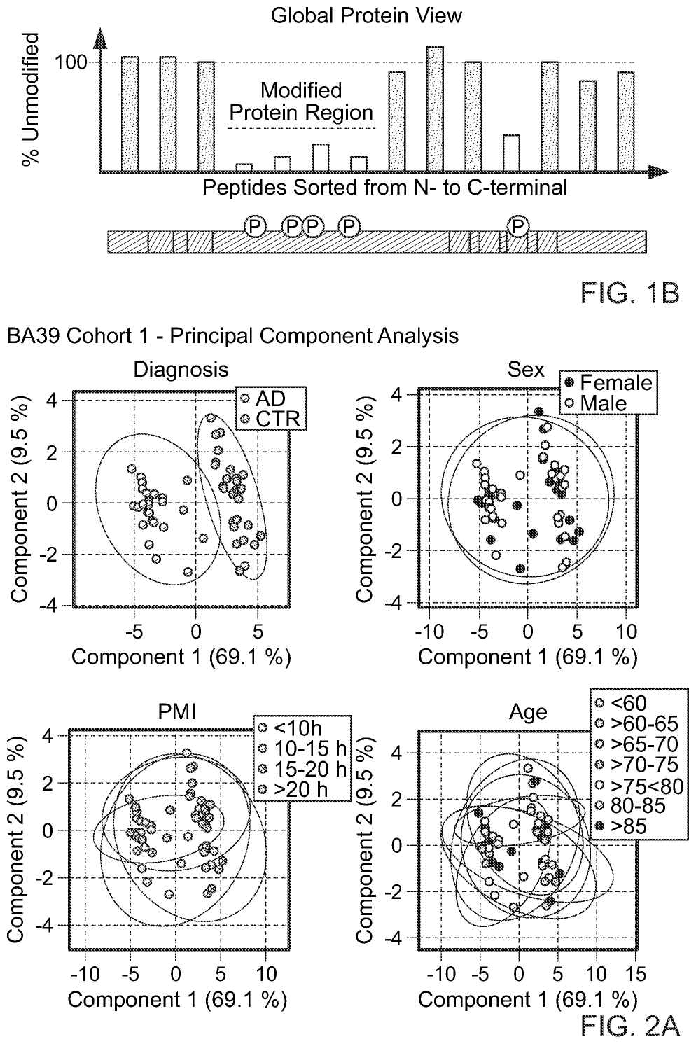

One exemplary workflow is shown in FIGS. 1A-1B. Referring to FIG. 1A, the heavy tau standard is generated using the FLEX vector in the presence of heavily labeled amino acids.

After purification, it is added to unlabeled endogenous sample in a ratio of approx. 1:1. A mix of three species of heavy tau standard and 3 species of endogenous tau proteins is assumed. 2/3 of the endogenous species are phosphorylated on a specific site. The protein mix is subjected to enzymatic digest and Liquid chromatography-mass spectrometry (LC-MS) analysis. All unmodified tau peptides will be present as pairs, featuring the light and the heavy isotopologue. While each pair of peptide species has varying signal intensities, the light-to-heavy (L/H) intensity ratio of all unmodified peptides reflects the initial mixing ratio (here, L/H=1 as an example). The phosphorylation causes a mass shift, reducing the amount of detectable unmodified peptide. In consequence, a deviation of the mixing ratio is observed (here, L/H=0.33 as an example). The extent of modification on that peptide can be inferred by the amount of ‘missing’ unmodified species (here, [Mod]=0.67 as an example). Referring to FIG. 1B, plotting of peptide L/H ratios sorted from protein N- to C-terminus allows for a global visualization of modified peptides and protein regions.

### Isotope-Labeled Samples

An exemplary method for preparing isotope-labeled (‘heavy’) tau proteins (e.g., Tau isoform 4R2N, GI: 294862262) comprise cloning the tau gene into a vector, and expressing the protein with isotopically labeled amino acids.

In some embodiments, expression of human Tau proteins can be carried out in bacteria or yeast expression system, e.g., from E. coli cells. In some embodiments, it can be translated in a cell-free expression system, e.g., wheat germ expression (WGE) system (Cell Free Sciences, Wheat Germ Expression H Kit-NA).

Expression can be carried out in the presence of one or more isotope labeled amino acids. Isotope labeled amino acids include, but are not limited to, e.g., lysine (13C6 15N2), arginine (13C6 15N4) and asparagine (13C4 15N1), etc.

In some embodiments, the isotopes for the isotopically labeled amino acids include ²H, ¹³C, ¹⁴C, ¹⁵N and ³³P, etc. Thus, the labeled amino acid residues are “heavier” as compared to unlabeled amino acid residues. However, other isotopes can be used, for example, some isotopes with less atomic mass. In those cases, the labeled amino residues will be “lighter” as compared to unlabeled amino acids.

Tau proteins can be purified by various means. In some embodiments, human Tau proteins can be purified by chromatography, e.g., cation exchange chromatography and/or size exclusion chromatography. In some embodiments, Ni-Sepharose beads are used to purify heavy tau standard. Briefly, after a prewash in binding buffer, beads are incubated with samples. After removal of the unbound fraction, beads are washed with wash buffer, followed by elution of tau.

### Selected Reaction Monitoring (SRM) and Parallel Reaction Monitoring (PRM)

The quality of the FLEXiTau data strongly depends on the sensitive and reproducible MS-based detection of the unmodified peptide species. To ensure this, in some embodiments, a targeted assay specifically tailored to monitor the unmodified tau using SRM is devised.

SRM is a mass spectrometry technique for the detection and quantification of specific, predetermined analytes with known fragmentation properties in complex backgrounds. SRM is used for precise quantification of targeted proteins (Kuhn 2014, Picotti 2009, Anderson 2006). It was originally used for the quantification of small molecules (such as metabolites or drugs (Zweigenbaum 2000). SRM is used most effectively in a liquid chromatography-coupled mass spectrometry (LC-MS) system, where a capillary chromatography column is connected in-line to the electrospray ionization source of the mass spectrometer. SRM exploits the unique capability of triple quadrupole (QQQ) (Yost 1979, Yost 1978) mass spectrometers to act as mass filters and to selectively monitor a specific analyte molecular ion and one or several fragment ions generated from the analyte by collisional dissociation (Yost 1979, Yost 1978, Kondrat 1978). The number of such fragment ions that reach the detector is counted over time, resulting in a chromatographic trace with retention time and signal intensity as coordinates. Several such precursor-fragment ion pairs, termed SRM transitions, can be sequentially and repeatedly measured at a periodicity that is fast compared to the analyte's chromatographic elution, yielding chromatographic peaks for each transition that allow for the concurrent quantification of multiple analytes. When multiplexing SRMs the assay is termed as a multiple reaction monitoring (MRM) assay, which is frequently used as a synonym of SRM. Parallel reaction monitoring (PRM) is the application of SRM with parallel detection of all transitions in a single analysis—this assay has recently been facilitated by the development of a high resolution mass spectrometers.

When applied to proteomics, SRM measures peptides produced by the enzymatic digestion of a proteome as surrogates of the corresponding proteins. Molecular ions within a mass range centered around the mass of the targeted peptide are selected in the first mass analyzer (Q1), fragmented at the peptide bonds by collision-activated dissociation (in Q2) and one or several of the fragment ions uniquely derived from the targeted peptide are measured by the second analyzer (Q3) (Kuhn 2014, Lange 2008). Integration of the chromatographic peaks for each transition supports the relative or, if suitable heavy isotope-labeled reference standards are used, absolute quantification of the targeted peptide(s) initially released from the protein and loaded on the LC-MS system. A suitably chosen set of SRM transitions therefore constitutes a specific assay to detect and quantify a target peptide and, by inference, a target protein in complex samples.

A crucial step in developing SRM assays is the identification of the most sensitive and selective transitions (pair of peptide and their fragment ion masses). A spectral library is created in order to find suitable transitions. To this end, high-resolution liquid chromatography tandem mass spectrometry (LC-MS/MS) of purified, digested tau standard is performed and it generates a collection of experimentally detected peptides and their fragment ions.

The spectral library is then used to develop a quantitative SRM assay for these peptides, choosing the transitions with highest intensity without interfering signals. The sensitivity of the SRM method can be maximized by acquisition of the transitions in a small retention time window (termed scheduled SRM). Therefore, in some embodiments, a scheduled 30 min LC-SRM method is developed. This method is suitable for pure/low complex tau samples and enables tau modification profile quantification from pure/low complex tau samples in a sensitive and time efficient manner. Methods of implementing SRM is described in various articles, e.g., Lange, Vinzenz; Picotti, Paola; Domon, Bruno; Aebersold, Ruedi (2008). “Selected reaction monitoring for quantitative proteomics: a tutorial”. Molecular Systems Biology. 4; Picotti, Paola, and Ruedi Aebersold. “Selected reaction monitoring-based proteomics: workflows, potential, pitfalls and future directions.” Nature methods 9.6 (2012): 555-566.

In some embodiments, parallel reaction monitoring is used to monitor the amount of modified and unmodified tau peptide fragments. Parallel reaction monitoring (PRM) is the application of SRM with parallel detection of all transitions in a single analysis using a high resolution mass spectrometer. Methods of implementing Parallel reaction monitoring is described in various articles, e.g., in Peterson, A. C.; Russell, J. D.; Bailey, D. J.; Westphall, M. S.; Coon, J. J. (2012). “Parallel Reaction Monitoring for High Resolution and High Mass Accuracy Quantitative, Targeted Proteomics”. Molecular & Cellular Proteomics. 11 (11): 1475-1488.

### Data-Independent Acquisition

While the use of SRM ensures optimum sensitivity, accuracy and precision, the analytes of interest have to be defined upfront because only those analytes are monitored with SRM. Mining data for other analytes, defined at a later point as being of interest, is not possible.

To provide such flexibility of mining data post hoc for analytes of interest, the present disclosure provides a totally unbiased mass spectrometric method to monitor all detectable tau-derived peptides using Data Independent Acquisition (DIA); also called “Sequential Window Acquisition of all Theoretical Mass Spectra” or SWATH) routines.

DIA is a mass spectrometry technique for the unbiased identification and quantification of all detectable analytes. In DIA, the first quandrupole is stepped through the entire m/z-range, selecting ranges of e.g. 25 m/z-units (400 to 425, 425 to 450, 450 to 475, etc.). In some embodiments of DIA, the individual m/z steps are adjusted in width according to the complexity within a given m/z range, so that every m/z step features a similar number of precursors. The relevant methods are described in, e.g., Gillet, Ludovic C., et al. “Targeted data extraction of the MS/MS spectra generated by data-independent acquisition: anew concept for consistent and accurate proteome analysis.” Molecular & Cellular Proteomics 11.6 (2012): 0111-016717; Law, Kai Pong, and Yoon Pin Lim. “Recent advances in mass spectrometry: data independent analysis and hyper reaction monitoring.” Expert review of proteomics 10.6 (2013): 551-566; Rosenberger, George, et al. “A repository of assays to quantify 10,000 human proteins by SWATH-MS.” Scientific data 1 (2014); Sidoli, Simone, et al. “Sequential Window Acquisition of all Theoretical Mass Spectra (SWATH) Analysis for characterization and quantification of histone post-translational modifications.” Molecular & Cellular Proteomics 14.9 (2015): 2420-2428; Chang, Rachel Yoon Kyung, et al. “SWATH analysis of the synaptic proteome in Alzheimer's disease.” Neurochemistry international 87 (2015): 1-12; Zhang, Ying, et al. “The Use of Variable Q1 Isolation Windows Improves Selectivity in LC-SWATH-MS Acquisition.” Journal of proteome research 14.10 (2015): 4359-4371; Aebersold, Ruedi, et al. “Applications and Developments in Targeted Proteomics: From SRM to DIA/SWATH.” Proteomics 16.15-16 (2016): 2065-2067; each of which is incorporated by reference in its entirety.

All precursors eluting at that moment off the liquid chromatography column within the selected m/z range are simultaneously fragmented and the fragment ions are detected in the second, the high resolution/high accuracy mass analyzer, i.e. Orbitrap or time-of-flight mass analyzer. Based on the elution profiles, the connectivity between the precursors and fragment ions are established. For the subsequent of the detected and fragmented peptides, spectral libraries are used, i.e. spectra for the analytes of interest have to be available in order to identify them. However, this also means if new analytes are identified as being of interest, the data can be re-interrogated with a new spectral library featuring also the spectrum of the novel analyte.

When analyzing samples by DIA, the quantification of the unmodified Tau-derived peptides (endogenous as well as exogenous, i.e. heavy isotope labeled peptides) are easily, accurately and precisely identified and quantified. Simultaneously, all detectable modified Tau-derived peptides are analyzed and fragmented so that they can be identified once an appropriate example fragment ion spectrum is obtained that can be used for the spectral library. Sophisticated quantification algorithms as provided by programs such as Skyline, Spectronaut, or OpenSWATH allows the subsequent quantification of the modified peptides. These quantification algorithms and the methods to use mass spectrometry are described in numerous articles, e.g., MacLean, Brendan, et al. “Skyline: an open source document editor for creating and analyzing targeted proteomics experiments.” Bioinformatics 26.7 (2010): 966-968; Schilling, Birgit, et al. “Platform-independent and label-free quantitation of proteomic data using MS1 extracted ion chromatograms in Skyline application to protein acetylation and phosphorylation.” Molecular & cellular proteomics 11.5 (2012): 202-214; Schubert, Olga T., et al. “Building high-quality assay libraries for targeted analysis of SWATH MS data.” Nature protocols 10.3 (2015): 426-441; Rardin, Matthew J., et al. “MS1 peptide ion intensity chromatograms in MS2 (SWATH) data independent acquisitions. Improving post acquisition analysis of proteomic experiments.” Molecular & Cellular Proteomics 14.9 (2015): 2405-2419; Bruderer, Roland, et al. “Extending the limits of quantitative proteome profiling with data-independent acquisition and application to acetaminophen-treated three-dimensional liver microtissues.” Molecular & Cellular Proteomics 14.5 (2015): 1400-1410; Bruderer, Roland, et al. “High-precision iRT prediction in the targeted analysis of data-independent acquisition and its impact on identification and quantitation”; Röst, Hannes L., et al. “OpenSWATH enables automated, targeted analysis of data-independent acquisition MS data.” Nature biotechnology 32.3 (2014): 219-223; each of which is incorporated by reference in its entirety.

### Classifiers

Classifiers are generated via a data processing system by applying one or more mathematical models to a dataset. In some embodiments, a classifier for each patient group is developed. For example, a classifier can be developed for Alzheimer's disease (AD), Argyrophilic grain disease (AGD), Corticobasal degeneration (CBD), Pick's disease (PiD) and Progressive supranuclear palsy (PSP).

In some embodiments, the input data include normalized L/H peptide intensity ratios of peptide. In some embodiments, a sample can be represented by the intensity ratios of 1, 2, 3, 4, 5, 6, 7, 8, 9, 10, 11, 12, 13, 14, 15, 16, 17, 18, 19, 20, or more than 20 peptides (features). In some embodiments, a sample is represented by a vector of 17 peptides (features). In some embodiments, the vector can include the absolute abundance.

In some embodiments, the computational classifier is designed for each disease category. In some embodiments, a binary dataset is created including the case category of interest and the reference category. In some embodiments, the reference category includes objects who do not have the disease of interest. In some embodiments, the reference category include subjects who do not have any tauopathies (e.g., all subjects in the reference category do not have a tauopathy). For example, a case category of interest can include all subjects have AD, and the reference category can include all non-AD subjects. In some other embodiments, the reference category only includes control subjects (subjects without tauopathies).

Mathematical models useful in accordance with the disclosure include those using both supervised and unsupervised learning techniques. In one embodiment, the mathematical model chosen uses supervised learning in conjunction with a training population to evaluate each possible combination of variables. Various mathematical models can be used, for example, a regression model, a logistic regression model, a neural network, a clustering model, principal component analysis, correlated component analysis, nearest neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, and genetic programming and weighted voting, etc. In some embodiments, the classifier involves a supervised machine learning model. In some embodiments, hierarchical clustering is used, e.g., based on Euclidean distance.

Many machine learning methods are available for supervised machine learning classifiers. These methods include, but are not limited to, Random forest (RF), Neural networks (Nnet), k-nearest neighbor (KNN), Learning Vector Quantization (LVQ), Linear Discriminant Analysis (LDA), and Support Vector Machines (SVM), etc. These algorithms are known in the art, and are described in various literature, e.g., Leo Breiman J F, Charles J. Stone, R. A. Olshens, J. Classification and Regression Trees. Wadsworth Statistics/Probability 1984; Bishop C M. Neural Networks for Pattern Recognition: Oxford: Oxford University Press; 1995; Altman N S. An Introduction to Kernel and Nearest-Neighbor Nonparametric Regression. The American Statistician 1992; 46(3): 175-85; Kohonen T. Learning vector quantization.: MIT Press; 1995; Fisher R A. THE USE OF MULTIPLE MEASUREMENTS IN TAXONOMIC PROBLEMS. Ann Eugen 1936; 7(2): 179-88; Cortes C, Vapnik V. Support-Vector Networks. Machine Learning 1995; 20(3): 273-97).

In some embodiments, a recursive feature elimination method based on the Random Forest (RF) algorithm is used to select the feature set that provides optimal separation of the case category and reference category in the training dataset.

Classifier can be evaluated using an independent testing dataset. This approach can be repeated for each case category, i.e. also PSP, PiD, CBD, and ctrl. The performance of the classifiers is assessed by accuracy (ac), defined as the total number of correctly classified cases (True Positives, TP, and True Negatives, TN) relative to the total number of cases in the testing set. Sensitivity (se) of the classifier is calculated as the number of TP divided by the total number of cases with given condition, that is TP and False Negatives (FN) (se=TP/(TP+FN)). Specificity is determined as the proportion of TN to the number of cases without given condition, that is TN plus False Positives (FP) (sp=TN/(TN+FP)). The performance (the positive diagnostic likelihood ratio) of a classifier, expressed by its true positive rate (TPR, or sensitivity), and false positive rate (FPR, or 1-specificity), is plotted in a receiver operator curve (ROC) space. The predictive power of each classifier can be further assessed by calculating the area under the ROC curve (AUC; AUC: 0.9-1.0=excellent; 0.8-0.9=good; 0.7-0.8=fair; 0.6-0.7=poor; 0.5-0.6=fail). Description of various statistics to evaluate the performance of the classifier can be found, e.g., in Sing T, Sander O, Beerenwinkel N, Lengauer T. ROCR: visualizing classifier performance in R. Bioinformatics 2005; 21(20): 3940-1. A perfect ROC area score of 1.0 is indicative of both 100% sensitivity and 100% specificity.

In some embodiments, classifiers are selected on the basis of the evaluation score. In some embodiments, the evaluation scoring system used is a receiver operating characteristic (ROC) curve score determined by the area under the ROC curve. In some embodiments, classifiers with scores of greater than 0.95, 0.9, 0.85, 0.8, 0.7, 0.65, 0.6, 0.55, or 0.5 are chosen. In some embodiments, where specificity is important to the use of the classifier, a sensitivity threshold can be set, and classifiers ranked on the basis of the specificity are chosen. For example, classifiers with a cutoff for specificity of greater than 0.95, 0.9, 0.85, 0.8, 0.7, 0.65, 0.6, 0.55 0.5 or 0.45 can be chosen. Similarly, the specificity threshold can be set, and classifiers ranked on the basis of sensitivity (e.g., greater than 0.95, 0.9, 0.85, 0.8, 0.7, 0.65, 0.6, 0.55 0.5 or 0.45) can be chosen. Thus, in some embodiments, only the top ten ranking classifiers, the top twenty ranking classifiers, or the top one hundred ranking classifiers are selected. The ROC curve can be calculated by various statistical tools, e.g., Statistical Analysis System (SAS®), and R (a language and environment for statistical computing and graphics).

A supervised classifier can be computed for patient categories of interest. In some embodiments, for the training of a classifier for a certain disease category a binary approach is used whereby the case category (for example AD) is classified against the remaining ‘mixed’ reference category (including all non-AD samples, e.g. CBD, PSP, PiD and ctrl). In some embodiments, the training process is repeated several times, for example, 5, 10, 15, 20, 50 times, i.e. each time a different subset of the reference category is randomly selected in order to obtain a stable classifier.

Generally, the training data set includes data obtained from a training population (e.g., a group of individuals whose diagnoses are determined). As described above, a data processing system applies a mathematical model to a training dataset and generates and trains a classifier. The classifier is the resultant mathematical model including the values for various parameters of the mathematical model. In turn, a data processing system applies one or more of these generated classifiers to a testing dataset for one or more test subjects to determine whether the test subject(s) have, or likely to have any tauopathies, e.g., Alzheimer's disease (AD), Argyrophilic grain disease (AGD), Corticobasal degeneration (CBD), Pick's disease (PiD) and Progressive supranuclear palsy (PSP).

Classifiers can be used alone or in combination with each other to create a formula for determining whether a subject has any tauopathies. One or more selected classifiers can be used to generate a formula. It is not necessary that the method used to generate the data for creating the formulas be the same method used to generate data from the test subject.

In some embodiments, the individuals of the training dataset used to derive the model or the classifier are different from the individuals of a population used to test the model or the classifier. As would be understood by a person skilled in the art, this allows a person skilled in the art to characterize an individual whose phenotypic trait characterization is unknown, for example, to determine the disease status of a subject, or the likelihood that an individual have a disease.

Applying a mathematical model to the data will generate one or more classifiers. In some embodiments, multiple classifiers are created that are satisfactory for the given purpose (e.g., all have sufficient AUC and/or sensitivity and/or specificity). In some embodiments, a formula is generated that utilizes more than one classifier. For example, a formula can be generated that utilizes classifiers in series. Other possible combinations and weightings of classifiers would be understood and are encompassed herein.

### Diagnostics

The methods described in this disclosure can be used for diagnosis, including in vivo and in vitro diagnostic tools. In some embodiments, the development of tau-based biomarkers in cerebrospinal fluid (CSF), plasma or brain biopsy tissue can be used for clinical diagnostics.

In some embodiments, the subject is suspected of having a tauopathy, e.g., Alzheimer's disease, progressive supranuclear palsy, corticobasal degeneration, Pick's disease. A sample containing tau proteins is collected from the subject. The extent of post translational modification is determined by the methods as described in the present disclosure, and a dataset is generated. A dataset can have one or more data records. A classifier is applied to the dataset to determine whether the subject has a tauopathy, or the likelihood that the subject has a tauopathy.

Provided herein are methods for diagnosing a tauopathy in a subject, the method comprising: (a) obtaining a biological sample from the subject; (b) identifying one or more post translational modifications (PTMs) in a tau protein, wherein the one or more PTMs are at positions selected from the group consisting of K24, Y29, T30, T39, K44, S46, S56, S61, S64, K67, S68, T69, T71, K87, T102, T111, S113, T153, T175, K180, T181, S184, S185, S191, S198, S199, S202, T205, S210, T212, S214, T217, T220, T231, S235, S237, S238, K240, S241, K254, K257, S258, K259, S262, T263, K267, K274, K280, K281, S289, K290, S293, K298, S305, Y310, K311, S316, K317, K321, K331, K343, K347, S352, K353, S356, T361, K369, K370, K375, K385, T386, Y394, K395, S396, S400, T403, S404, R406, S409, S412, S413, T414, S416, S422, S433, S435, K436, and K438 (based on numbering on human 2N4R isoform), thereby diagnosing the tauopathy in the subject

Table 1 lists examples of PTMs at amino acid residues in human tau protein (2N4R isoform). In some embodiments, the detection of the 0N and/or 4R isoforms indicates that the subject is at risk of developing of AD.

In some embodiments, phosphorylation(s) at least 1, 2, or all of the positions S199, S202 and T205 indicates a higher risk of developing AD, or the presence of AD. In some embodiments, phosphorylation(s) at least 1, 2, 3, or all of the positions S198, S199, 5202, and T205 indicates a higher risk of developing AD, or the presence of AD. In some embodiments, phosphorylation(s) at least 1, 2, or all of the positions S212, S214, and S217 indicates a higher risk of developing AD, or the presence of AD.

In some embodiments, ubiquitination(s) at least 1, 2, 3, 4, 5, 6, 7, 8, 9 10, or all of the positions K180, K240, K254, K257, K259, K267, K274, K281, K290, K298, K311, K317, K321, K343, K353, K369, and K395 indicates a higher risk of developing AD, or the presence of AD.

In some embodiments, acetylation(s) at least 1, 2, 3, or all of the positions K353, K369, K370, and K375 indicates a higher risk of developing AD, or the presence of AD.

In some embodiments, phosphorylation(s) at T181 and/or T231 indicates a lower risk of developing AD, or the absence of AD.

In some embodiments, ubiquitination at any one or both of K311 and K317, and/or phosphorylation at any one or both of T217 and S262 can be used to differentiate between AD patients and subjects without AD. In some embodiments, ubiquitination(s) at any one or both of K311 and K317 indicates a higher risk of developing AD, or the presence of AD.

In some embodiments, ubiquitination or acetylation at least 1, 2, 3, or all of the positions K311, K317 K321 and K369 indicates a higher risk of developing AD, or the presence of AD.

In some embodiments, the methods described in the present disclosure can be used for post-mortem classification of tissues and re-evaluation. In some embodiments, these samples are from brain banks.

Tau Peptides with PTMs

Table 2 lists peptides detected with their sequence being ordered by their amino acid location in tau (N- to C-terminal). Peptide modification extent is determined by the difference of normalized L/H ratio to ctrl-tau, where 100% represents a peptide that is fully modified (no unmodified peptide detected). P-values were calculated in comparison to control tau (student t-test). Corresponding phosphorylation sites detected by complementary LC-MS/MS analysis are also shown.

Peptide fragments with the modifications as listed in Table 2 can be used as antigens. Antibody or antibody fragments that immunospecifically bind to these antigens can be used for various purposes, e.g., diagnosis and treatment.

In some embodiments, the amount of PTMs of peptide for these peptide fragments can be used in a classifier to determine whether a test subject has a tauopathy, the method includes the steps of inputting, into a classifier, data representing the amount of post translational modifications (PTMs) for a set of tau protein peptide fragments from a test subject, wherein the classifier being for determining whether the amount of PTMs for the set of tau protein peptide fragments classifies with (A) a set of data repressing the amount of PTMs for the set of tau protein peptide fragments from a first group of individuals who have the tauopathy; as opposed to classifying with (B) a set of data repressing the amount of PTMs for the set of tau protein peptide fragments from a second group of individuals who does not have the tauopathy; applying, by the one or more data processing devices, the classifier to the data representing the amount of PTMs for the set of tau protein peptide fragments from the test subject; and determining whether the test subject is classified with the first group of individuals who have the tauopathy or the second group of individuals who do not have the tauopathy.

In some embodiments, evaluating a subject for having a tauopathy involves determining whether determine whether the level of tau PTM associated with tau peptide fragments in a sample from the test subject is are significantly altered relative to the level for each tau peptide fragment in a reference group. In some embodiments, the reference group is a control group (e.g., a group of subjects who do not have tauopathies). In some embodiments, the reference group includes all subjects who does not have the tauopathy of interest (e.g., AD), and these subjects may have some other diseases (e.g., PSP).

In one aspect, the disclosure provides a computer-implemented method for determining whether a test subject has a tauopathy. In one embodiment, the method comprises, inputting, into a classifier, data representing the amount of post translational modifications (PTMs) for a set of tau protein peptide fragments from a test subject, wherein the classifier being for determining whether the amount of PTMs for the set of tau protein peptide fragments classifies with (A) a set of data repressing the amount of PTMs for the set of tau protein peptide fragments from a first group of individuals who have the tauopathy; as opposed to classifying with (B) a set of data repressing the amount of PTMs for the set of tau protein peptide fragments from a second group of individuals who does not have the tauopathy; applying, by the one or more data processing devices, the classifier to the data representing the amount of PTMs for the set of tau protein peptide fragments from the test subject; and determining whether the test subject is classified with the first group of individuals who have the tauopathy or the second group of individuals who do not have the tauopathy. In some embodiments, the set of the tau peptide fragments comprises one or more fragments listed in Table 2.

In some embodiments, the classifier is based on Random forest (RF), Neural networks (Nnet), k-nearest neighbor (KNN), Learning Vector Quantization (LVQ), Linear Discriminant Analysis (LDA), and Support Vector Machines (SVM).

In some embodiments, the tauopathy is selected from the group consisting of Alzheimer's disease (AD), Argyrophilic grain disease (AGD), Corticobasal degeneration (CBD), Pick's disease (PiD) and Progressive supranuclear palsy (PSP).

In some embodiments, stochastic gradient descent is used in the classifier.

### Antibodies and Antibody Fragments

Peptides that immunospecifically bind to tau proteins with PTMs can be prepared from immune cells and molecular biology techniques. In some embodiments, provided herein are antibodies, or antigen binding fragments thereof, targeting one or more tau peptide fragments of a tau protein having one or more PTMs. In some embodiments, these peptides do not bind to tau proteins without such PTMs. In some embodiments, the antibodies or antigen-binding fragments specifically target tau proteins with ubiquitination/acetylation at K311, K317 K321 and/or K369. Because these PTMs are charge neutralizing PTMs and can reduce the kinetic barriers to filament formation, antibodies or antigen-binding fragments targeting these PTMs can inhibit filament formation.

Also provided herein are methods of using an antibody, or antigen binding fragments thereof, to detect one or more PTMs. In some embodiments, the antibody targets S262. In some embodiments, the antibody targets S356. Any suitable antibodies can be used in the methods described herein. In some embodiments, the antibody is selected from the group consisting of MIgG, Tau12, HT7, Tau5, Tau7, AT8, AT100, AT180, AT270, PHF1, RN235, FK2, R23, DA9, HL5, and HL12. In some embodiments, the antibodies described herein can be used for treating a tauopathy in a subject. In some embodiments, the antibodies described herein can be used for reducing the seeding activity of a tau protein or peptide in a subject.

Examples of antibodies that bind to Tau and their epitopes are shown in Table 3 and Table 4. The detailed information of these antibodies can be accessed through sources such as the suppliers' websites. In some embodiments, the antibodies described herein has a heavy chain variable region (VH) comprising VH CDR1, VH CDR2, and VH CDR3 that are at least 70%, 75%, 80%, 85%, 90%, 95%, or 100% identical to VH CDR1, VH CDR2, and VH CDR3 of any one of the antibodies listed in Table 3 and Table 4, in any sequence.

In some embodiments, the antibodies described herein has a light chain variable region (VL) comprising VL CDR1, VL CDR2, and VL CDR3 that are at least 70%, 75%, 80%, 85%, 90%, 95%, or 100% identical to VL CDR1, VL CDR2, and VL CDR3 of any one of the antibodies listed in Table 3 and Table 4, in any sequence.

In some embodiments, the antibodies described herein has a heavy chain variable region (VH) that is at least 70%, 75%, 80%, 85%, 90%, 95%, or 100% identical to the VH sequence of any one of the antibodies listed in Table 3 and Table 4.

In some embodiments, the antibodies described herein has a light chain variable region (VL) that is at least 70%, 75%, 80%, 85%, 90%, 95%, or 100% identical to the VL sequence of any one of the antibodies listed in Table 3 and Table 4.

In some embodiments, the antibodies described herein bind to the same epitope as any one of the antibodies listed in Table 3 and Table 4.

In some embodiments, the antibodies described herein cross competes with any one of the antibodies listed in Table 3 and Table 4.

In some embodiments, the antibodies described herein are humanized antibodies.

TABLE 3
Commercial Tau antibodies

| Name | Supplier | Catalog | Target/epitope |
|---|---|---|---|
| Tau12 | Millipore Sigma | MAB2241 | Pan-tau, N-term, a.a. 12-24 |
| Tau5 | Thermo Fischer | AHB0042 | Pan-tau, a.a. 218-225 |
| | Scientific | | |
| HT7 | Thermo Fischer | MN1000 | Pan-tau, a.a. 159-163 |
| | Scientific | | |
| Tau7 | Millipore Sigma | MAB2239 | Pan-tau, C-term, a.a. |
| | | | 430-441 |
| AT270 | ThermoFisher | MN1050 | Phospho-Tau (Thr181) |
| | Scientific | | |
| AT8 | ThermoFisher | MN1020 | Phospho-Tau (Ser202, |
| | Scientific | | Thr205) |
| AT100 | ThermoFisher | MN1060 | Phospho-Tau (Thr212, |
| | Scientific | | Ser214) |
| pT217 | Abcam | ab192665 | Phospho-Tau (Thr217) |
| AT180 | ThermoFisher | MN1040 | Phospho-Tau (Thr231) |
| | Scientific | | |
| RN235 | Millipore Sigma | MABN2275- | Phospho-Tau (Ser235) |
| | | 100UG | |
| pS262 | ThermoFisher | 44-750G | Phospho-Tau (Ser262) |
| | Scientific | | |
| pS356 | Abcam | ab51036 | Phospho-Tau (Ser356) |
| EPR2731 | Abcam | ab109390 | Phospho-Tau (Ser396) |
| FK2 | Enzo | BML- | Mono- & Poly-ubiquitin |
| | | PW8810- | conjugates |
| | | 0100 | |

TABLE 4
Non-commercial Tau antibodies

| Name | Supplier | Catalog | Target/epitope |
|---|---|---|---|
| HL5 mouse IgG1 | Peter Davies | NA | Unknown |
| HL12 mouse IgG1 | Peter Davies | NA | Phospho-Tau (Thr50) |
| DA9 mouse IgG1 | Peter Davies | NA | Pan-tau, a.a. 102-149 |
| R23 mouse IgG1 | Peter Davies | NA | Phospho-Tau (Thr231) |
| PHF1 | Peter Davies | NA | Phospho-Tau (Ser396/S404 |
| 12E8 | Peter | NA | Phospho-Tau (Ser 262 |
| | Seubert/Elan | | KXGS motif and/or |
| | | | Ser356) |

In some embodiments, the antibody described herein is used for diagnosing and/or treating a tauopathy, e.g., Alzheimer's Disease (AD). In some embodiments, the antibody reverses or prevents fibrillization and/or seeding of Tau peptides. In some embodiments, the antibody reverses or prevents aggregation of Tau peptides.

The method herein involves preparation of peptides directed against one or more different antigens. In some embodiments, the antigen is a full length tau protein with one or more PTMs of interest. In some embodiments, the antigen is a tau peptide fragment with one or more PTMs of interest.

In some embodiments, an animal or host to be immunized with the antigens is selected. In the preferred embodiment, the animal is a rodent, e.g. a mouse. The amount of antigen of interest administered to the host animal may, for example, range from about 0.01 μg to about 250 μg, preferably from about 0.1 μg to about 100 μg. Where the primary response is weak, it may be desirable to boost the animal at spaced intervals until the antibody titer increases or plateaus. After immunization, samples of serum (test bleeds) may be taken to check the production of specific antibodies. Preferably, the host animal is given a final boost about 3-5 days prior to isolation of immune cells from the host animal. Monoclonal antibodies may be made using the hybridoma method first described by Kohler et al., Nature, 256:495 (1975). In the hybridoma method, “immune cells” that produce or are capable of producing polyclonal antibodies are obtained from the animal immunized as described above. Various immune cells are described above, with lymph nodes or spleen being the preferred source of immune cells for generating monoclonal antibodies. Such cells may then be fused with myeloma cells using a suitable “fusing agent”, such as polyethylene glycol or Sendai virus, to form a hybridoma cell. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium such as HAT medium.

In some embodiments, obtaining or targeting immune cells can include one or more and/or combinations of, for example: obtaining or providing an antigen (e.g., peptide fragments with modifications as shown in Table 1) that can bind (e.g., bind specifically) to a target immune cell; contacting the antigen with a sample; detecting the antigen; determining whether the antigen is bound to a target immune cell; and, if the antigen is bound to a target immune cell, then obtaining the target immune cell.

Methods for isolating or purifying genetic material (e.g., DNA and/or mRNA) from the obtained target immune cell are known in the art and are exemplified herein. Once such genetic material has been obtained, methods for using it to produce the therapeutic compositions disclosed herein are known in the art and/or are summarized below. Genetic material can be varied, using techniques known in the art to create polypeptide variants disclosed herein. Generating polypeptides from nucleic acids (e.g., cDNA) contained within or obtained from the target cell can include, for example, analysis, e.g., sequencing of heavy and light chain variable domains from target immune cells (e.g., single or isolated identified target immune cells).

In some embodiments, methods can include generating fully human antibodies, or fragments thereof (e.g., as disclosed above), and humanization of nonhuman antibodies. DNA can be readily isolated and/or sequenced from the obtained immune cells using conventional procedures (e.g., by using oligonucleotide probes that are capable of binding specifically to genes encoding the heavy and light chains of murine antibodies).

Once isolated, DNA can be placed into expression vectors, which are then transfected into host cells such as Escherichia coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 2 6-262 (1993) and Pluckthun, Immunol. Revs., 130:1 1-188 (1992).

Recombinant expression of an antibody or variant thereof generally requires construction of an expression vector containing a polynucleotide that encodes the antibody. The invention, thus, provides replicable vectors comprising a nucleotide sequence encoding an antibody molecule, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., U.S. Pat. Nos. 5,981,216; 5,591,639; 5,658,759 and 5,122,464) and the variable domain of the antibody may be cloned into such a vector for expression of the entire heavy, the entire light chain, or both the entire heavy and light chains.

Once the expression vector is transferred to a host cell by conventional techniques, the transfected cells are then cultured by conventional techniques to produce an antibody. Thus, the invention includes host cells containing a polynucleotide encoding an antibody of the invention or fragments thereof, or a heavy or light chain thereof, or portion thereof, or a single-chain antibody of the invention, operably linked to a heterologous promoter. In certain embodiments for the expression of double-chained antibodies, vectors encoding both the heavy and light chains may be co-expressed in the host cell for expression of the entire immunoglobulin molecule, as detailed below.

Mammalian cell lines available as hosts for expression of recombinant antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any functional immunoglobulin chains), SP20, CRL7030 and HsS78Bst cells. In some embodiments, human cell lines developed by immortalizing human lymphocytes can be used to recombinantly produce monoclonal antibodies.

### Therapeutic and Diagnostic Use

The disclosure provides methods of treatment that include administering to a subject a composition disclosed herein.

In some embodiments, subject selection can include obtaining a sample from a subject (e.g., a candidate subject) and testing the sample for an indication that the subject is suitable for selection. In some embodiments, the subject can be confirmed or identified, e.g. by a health care professional, as having had or having a condition or disease. In some embodiments, exhibition of a positive response towards a condition or disease can be made from patient records, family history, and/or detecting an indication of a positive response. In some embodiments multiple parties can be included in subject selection. For example, a first party can obtain a sample from a candidate subject and a second party can test the sample. In some embodiments, subjects can be selected and/or referred by a medical practitioner (e.g., a general practitioner). In some embodiments, subject selection can include obtaining a sample from a selected subject and storing the sample and/or using the in the methods disclosed herein.

In some embodiments, the composition disclosed herein can be used for treating various tauopathies. For example, an antibody or an antibody fragment thereof that specifically targets tau protein with one or more specific PTMs can be used to treat a tauopathy, if the tauopathy is associated with tau proteins having these specific PTMs.

In some embodiments, the antibodies or the fragments thereof can be used in imaging agents. These imaging agents can target tau proteins with specific modification as described in this disclosure. In some embodiments, these in vivo biomarkers and imaging reagents can be used for diagnosis and prognosis, e.g. for staging disease and to measure efficacy of treatment in clinical trials.

In some embodiments, the antibodies or the fragments thereof can be used for various diagnosis purpose. In some embodiments, a sample is collected from a subject. An antibody or antibody fragment thereof that specifically target one or more PTMs of interest can be used to determine whether the tau protein in the subject has PTMs of interest. In some embodiments, the PTMs of interest may be associated with a tauopathy. If it is determined that the subject has the PTMs of interest, then the subject is determined to have the tauopathy (e.g., Alzheimer's disease (AD), Argyrophilic grain disease (AGD), Corticobasal degeneration (CBD), Pick's disease (PiD) and Progressive supranuclear palsy (PSP)).

In one aspect, the disclosure provides a kit comprising one or more agents as described herein for therapeutic and/or diagnostic purposes.

### Methods of Treatment

Provided herein are methods for treating a tauopathy in a subject, the method comprising administering an effective amount of a therapeutic agent that targets the one or more tau peptide fragments to the subject, thereby treating the tauopathy. In some embodiments, the therapeutic agent specifically binds to one or more tau peptide fragments with one or more post translational modification (PTM) as described herein. In some embodiments, the one or more PTM is located on the position selected from the positions listed in Table 1.

The one or more PTMs of the tau protein can be any one or more PTMs described herein. In some embodiments, the one or more PTM is selected from the group consisting of a phosphorylation at T231, a phosphorylation at S235, a phosphorylation at S237, a phosphorylation at S238, a ubiquitination at K311 and a ubiquitination at K317. In some embodiments, the one or more PTM is a phosphorylation at T231 and S235. In some embodiments, the one or more PTM is a phosphorylation at T231 and S237. In some embodiments, the one or more PTM is a phosphorylation at T231 and S238. In some embodiments, the one or more PTM is a ubiquitination at K311. In some embodiments, the one or more PTM is a ubiquitination at K317. In some embodiments, the one or more PTM is a ubiquitination at K311 and a ubiquitination at K317.

In some embodiments, the method comprises (a) obtaining a biological sample from the subject; (b) identifying one or more post translational modification (PTM) associated with one or more tau peptide fragments of a tau protein in the biological sample, wherein the one or more PTM is located on the position selected from the positions listed in Table 1; (c) administering an effective amount of a therapeutic agent that targets the one or more tau peptide fragments, thereby treating the tauopathy.

Treatment of a disease (e.g., a tauopathy) or individual (e.g., a subject having Alzheimer's Disease) according to the methods described herein is an approach for obtaining beneficial or desired medical results, including clinical results, but not necessarily a cure. For purposes of the methods described herein, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment. A treatment can include administration of one or more therapeutic agent.

In some embodiments, the treatment includes reducing or eliminating the seeding activity of a tau protein or peptide. Accordingly, also provided herein are methods of reducing or eliminating a seeding activity of a Tau peptide in a subject, the method comprising: administering an effective amount of a therapeutic agent that targets the one or more tau peptide fragments to the subject. In some embodiments, the method comprises (a) obtaining a biological sample from the subject; (b) identifying one or more post translational modification (PTM) associated with one or more tau peptide fragments of a tau protein in the biological sample, wherein the one or more PTM is located on the position selected from the positions listed in Table 1; (c) administering an effective amount of a therapeutic agent that targets the one or more tau peptide fragments, thereby reducing or eliminating a seeding activity of a Tau peptide in the subject.

In some embodiments, the seeding activity of a tau protein or peptide is reduced by at least 10%, 20%, 30%, 40%, 50%, 60%, 70%, 80%, 85%, 90%, 95% in the subject compared to the seeding activity of the tau protein or peptide without the administration of the therapeutic agent. In some embodiments, the seeding activity is completely eliminated to a non-detectable level.

In some embodiments, the therapeutic agent is an antibody or antigen-binding fragment thereof that binds to a Tau peptide. In some embodiments, the antibody is any antibody described herein. In some embodiments, the antibody is selected from the antibodies listed in Table 3.

In some embodiments, the antibody has the one or more CDR(s) comprising an amino acid sequence that is at least 80% identical to one or more CDR(s) of any one of the antibodies listed in Table 3.

The methods of treatments described herein can be used in combination with other suitable therapeutics for tauopathies. Suitable therapies for tauopathies, e.g., Alzheimer's Disease are known in the art.

The methods described herein include methods for the treatment of disorders associated with tauopathies (e.g., AD, AGD, CBD, PiD, PSP). In some embodiments, the disorder is Alzheimer's disease. Generally, the methods include administering a therapeutically effective amount of a composition as described herein (e.g., antibody or antibody fragment thereof), to a subject who is in need of, or who has been determined to be in need of, such treatment.

As used in this context, to “treat” means to ameliorate at least one symptom of the disorder associated with tauopathies. Often, the treatment results in improvement of symptoms. In some embodiments, a treatment can result in a reduction in tau protein aggregation.

In some embodiments, the treatment reduces the risk of developing disorders associated with tauopathies (e.g., AD, AGD, CBD, PiD, PSP). Generally, the methods include administering a therapeutically effective amount of a composition as described herein (e.g., antibody or antibody fragment thereof), to a subject who is determined to have a risk of developing disorders associated with tauopathies (e.g., AD, AGD, CBD, PiD, PSP). In some embodiments, the subjects have some early symptoms for tauopathies, e.g., changes in personality, behavior, sleep patterns, and executive function, memory loss, confusion, inability to learn new things, and difficulty carrying out multistep tasks, etc.

In some embodiments, the methods described herein further comprises performing one or more additional test on the subject. The additional tests include, but are not limited to, a behavioral test, a neurological exam, a brain imaging, a mental status test, a dementia test, and mood assessment.

### Pharmaceutical Compositions

The methods described herein include the use of pharmaceutical compositions comprising a polypeptide that immunospecifically binds the tau proteins with PTMs as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art (see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active composition in the required amount in an appropriate solvent with one or a combination of ingredients enumerated above, as required, followed by filtered sterilization. Generally, dispersions are prepared by incorporating the active composition into a sterile vehicle, which contains a basic dispersion medium and the required other ingredients from those enumerated above.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of an active agent (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Methods of Screening

Included herein are methods for screening test compounds, e.g., polypeptides, polynucleotides, inorganic or organic large or small molecule test compounds, to identify agents useful in the treatment of disorders associated with tau protein aggregation, and tauopathies, e.g., Alzheimer's Disease (AD), Progressive Supranuclear Palsy (PSP), Corticobasal Degeneration (CBD), Pick's Disease (PiD), Argyrophilic grain disease (AGD), Frontotemporal dementia and Parkinsonism associated with chromosome 17 (FTDP-17), Parkinson's disease, stroke, traumatic brain injury, mild cognitive impairment and the like. Agents useful in the treatment of disorders associated with tau protein aggregation include, for example, compounds, e.g., polypeptides, such as an antibody or other antigen binding molecule, polynucleotides, inorganic or organic large or small molecule compounds that bind to one or more tau PTM and/or inhibit association of PTM tau proteins.

In some embodiments, the methods for screening described herein include characterizing therapeutic agents, e.g., antibodies, using the platforms described herein (e.g., FLEXITau) and methods known in the art such as mass spectrometry. The screening methods provide precise information on target engagement of antibodies tested. For example, the methods define target engagement with respect to specificity of antibodies to the pathological Tau signature identified (e.g., PTMs of Tau protein or peptide). The screening methods describe described herein can further include, e.g., immunoreactivity assay of AD sarkosyl insoluble material, immunoprecipitation experiments of the antibodies, immunoblotting experiments of IPs from pooled AD lysate. Furthermore, seeding activity of immunodepeleted lysates, and IP eluates can be assessed. The immunodepleted lysates and IP eluates can be further analyzed by MS. The platforms described herein (e.g., FLEXITau) can also be used to measure modified and unmodified Tau peptides in the IP eluates.

Furthermore, high resolution immunohistochemistry can be used to catalogue the cellular distribution of Tau protein proteoforms identified by the screened agents (e.g., antibodies). Antibodies selected based on mass spectrometry characterization studies can be evaluated using immunohistochemistry.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czamik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a protein sample, a cell or living tissue or organ, and one or more effects of the test compound is evaluated. In a cultured or primary cell for example, the ability of the test compound to inhibit the PTM of interest or promote the PTM of interest is determined.

In some embodiments, the test sample is, or is derived from (e.g., a sample taken from) an in vivo model of a disorder as described herein. For example, an animal model, e.g., a rodent such as a rat, can be used.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modem genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on PTMs. Ability to modulate PTMs can be evaluated, e.g., using methods as described in this disclosure.

A test compound that has been screened by a method described herein and determined to inhibit PTMs of interest, or inhibit tau protein aggregation, or promote the PTM of interest can be considered a candidate compound. A candidate compound that has been screened, e.g., in an in vivo model of a disorder, e.g., AD, PSP, CBD, PiD, AGD, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that have the ability to inhibit certain PTMs, promote certain PTM, or inhibit tau protein aggregations) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating tauopathies, e.g., AD, AGD, CBD, PiD, PSP, or symptoms associated with tauopathies. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a tauopathy (e.g., AD, AGD, CBD, PiD, PSP), as described herein. The animal can be monitored for a change in the disorder, e.g., for an improvement in a parameter of the disorder, e.g., a parameter related to clinical outcome. In some embodiments, the parameter is memory, and an improvement would be an increase in short-term memory.

### EXAMPLES

The invention is further described in the following examples, which do not limit the scope of the invention described in the claims.

### Example 1: Materials and Methods

The materials and methods for preparation of brain tissue samples and mass spectrometry analysis described in this Example were used in the following Examples in this section.

### Selection of Human Alzheimer's Disease Patient and Non-Demented Control Samples

Human post-mortem parietal cortex (Brodmann area, BA39—angular gyrus) and frontal gyrus (BA46) specimens from patients with AD and non-demented age-matched controls were obtained from 5 different brain banks: 1) the Neurodegenerative Disease Brain Bank (NDBB), Memory and Aging Center, University of California, San Francisco (UCSF), CA; 2) the University of Maryland Brain & Tissue Bank at the University of Maryland School of Medicine, Baltimore, MD; 3) the Harvard Brain Tissue Resource Center, McLean Hospital, Harvard Medical School, Belmont, MA; 4) the University of Miami (UM) Brain Endowment Bank, Miller School of Medicine, Miami, MD; 5) the Human Brain and Spinal Fluid Resource Center (HBSFRC), VA West Los Angeles Healthcare Center, Los Angeles, CA. Tissue from brain banks 2) to 5) were acquired through the NIH NeuroBioBank (U.S. Department of Health and Human Services, National Institutes of Health). Pathological and clinical information, if available, was de-identified.

### Preparation of Brain Tissue Samples for MS

While still frozen, 0.25-0.35 g sections of cortical brain specimens were homogenized in 5 volumes lysis buffer (25 mM Tris-HCl buffer, pH 7.4, containing 150 mM NaCl, 10 mM ethylene diamine tetra acetic acid (EDTA), 10 mM EGTA, 1 mM DTT, 10 mM nicotinamide, 2 μM trichostatin A, phosphatase inhibitor cocktail (Sigma), and protease inhibitor cocktail (Roche)), using Precellys® tissue homogenizer. To obtain insoluble Tau fractions, sarkosyl fractionation was performed. Therefore, crude brain homogenates were clarified by centrifugation at 11,000×g for 30 min at 4° C. Part of the crude Tau fraction was treated with sarkosyl (1% final concentration) for 60 min at 4° C. and ultracentrifuged at 100,000 g for 2 h at 4° C. The supernatant was transferred to a new tube (sarkosyl-soluble fraction). The sarkosyl-insoluble pellet was carefully washed twice with 10 μl ddH₂O, air-dried, and solubilized in 100 μl 50 mM Tris buffer containing 1% SDS, 10 mM nicotinamide, 2 μM trichostatin A, and phosphatase and protease inhibitor cocktail. The protein concentration in the extracts was determined by bicinchoninic acid assay (BCA Protein Assay Kit, Thermo Scientific).

To quantify absolute Tau amounts and determine the extent of Tau modifications, sarkosyl-insoluble and soluble fractions were processed using the FLEXITau workflow (see, e.g., Mair W et al., Anal Chem. 2016 Apr. 5; 88(7):3704-14). FLEXITau is an MS-based strategy that is based on the addition of a full-length Tau protein standard containing the N-terminally tagged artificial tryptic FLEX-peptide to the biological sample of interest. Light FLEX-peptide is added in predetermined concentration to calculate absolute quantity of endogenous Tau. The relative peptide abundance of light and heavy Tau peptides can be used to infer modification extent of Tau for each peptide. In brief, Tau was in vitro transcribed and translated in a cell-free wheat germ expression (WGE) system according to the manufacturer's protocols (Cell Free Sciences, Wheat Germ Expression H Kit-NA). in the presence of heavy isotope (i.e. ¹³C and ¹⁵N) labeled lysine, arginine and aspartate and subsequently purified using Ni-Sepharose beads (Ni-Sepharose High Performance resin, GE Healthcare). Briefly, after a prewash in binding buffer (20 mM phosphate buffer, pH7.5, 500 mM NaCl, 10 mM imidazole) beads were incubated with WGE (ratio 1:4) for 1h rotating head-over-head at 4° C. for binding. After removal of the unbound fraction, beads were washed once with 1× volume and 3 times with 10× volume wash buffer (20 mM phosphate buffer, pH7.5, 500 mM NaCl, 10 mM imidazole). Elution of Tau was carried out in three consecutive steps (binding buffer with 100/300/500 mM imidazole, respectively). Success of enrichment was verified by SDS-PAGE and western blot analysis (data not shown). Pooled eluates were stored at −20° C.

Purified heavy Tau standard or sarkosyl-insoluble Tau fractions were diluted with 8 M urea and processed separately using Filter-Aided Sample Preparation (FASP) (FASP Protein Digestion Kit, Expedeon) with DTT as reduction agent and 1% acrylamide for cysteine alkylation. Protein mixtures were digested with 12.5 ng/μl trypsin (sequencing grade modified trypsin, Promega, Madison, WI) overnight at 37° C. Acidified peptides were desalted using C18 extraction plates (Waters). Vacuum-dried peptides were reconstituted in sample buffer (5% formic acid, 5% acetonitrile (ACN)) containing indexed retention time (iRT) peptides (Biognosys) and 50 fmol/μl non-labeled FLEX-peptide SENLYFQGDISR, synthesized by Sigma Life Science, quantified via amino acid analysis of Molecular Biology Core Facilities, Dana Farber Cancer Institute, Boston, MA). Heavy Tau standard peptides were added to insoluble (light) Tau peptides to achieve approximately a 1:1 ratio of Light-to-Heavy (L/H) tau.

### FLEXITau Measurements and Data Analysis

LC-SRM measurements of Tau L/H peptide ratios were performed (see, e.g., Mair W, et al., Anal Chem. 2016 Apr. 5; 88(7):3704-14). The FLEXITau SRM assay was optimized for the analysis of post-mortem tissue, guided by an extensive list of validated transitions generated in-house through LC-MS/MS analysis of sarkosyl-insoluble Tau on a quadrupole Orbitrap tandem mass spectrometer (Q Exactive, Thermo Fisher Scientific). After optimization of the transition list, peptide mixtures were analyzed on a triple quadrupole mass spectrometer (5500 QTRAP, Sciex) using a micro-autosampler AS3 and a nanoflow UPLC pump (both Eksigent/Sciex), using the trap-elute chip system (cHiPLC nanoflex, Eksigent). Briefly, peptides were first loaded onto the trap-chip (200 μm×75 μm, ChromXP C18-CL 3 μm 120 A, Nano cHiPLC Eksigent) and then separated using a 120 min gradient from 95% buffer A (0.1% (v/v) formic acid in HPLC-H₂O) and 5% buffer B (0.2% (v/v) formic acid in ACN) to 35% buffer B on the analytical column-chip (75 μm×15 cm, ChromXP C18-CL 3 μm 120 A, Nano cHiPLC Eksigent). The retention time window was set to 5 min and total scan time to 1.2 s, which ensured a dwell time over 20 ms per transition. To avoid sample carry-over, blanks were analyzed between every SRM run. To ensure no bias in acquisition, samples were run in randomized order (three technical replicates per sample). SRM data were analyzed and validated in Skyline (version 2.6, MacCoss Lab Software, University of Washington, Seattle, WA) (see., e.g., MacLean B, et al., Bioinformatics. 2010 Apr. 1; 26(7):966-8). All peptide transitions were evaluated for variability, similarity between y-ion ratios, elution times, and interfering signals by manual analysis. Peak boundaries were manually inspected and reassigned as needed to ensure correct peak detection and accurate integration. Peptides were considered ‘quantifiable’ if the peptide transitions had a signal-to-noise of >3 and at least three light and three heavy high-quality SRM transitions were observed. Peptides were kept for further downstream analysis if quantifiable in every patient sample. The final peptide list consisted of 17 Tau peptides. To compensate for differences in mixing ratio, samples were normalized by the L/H ratio of the least modified peptides. To this end, in each sample, the L/H ratio of peak intensities of each peptide was divided by the average of the three Tau peptides with highest ratio in that sample. Absolute abundance of Tau was calculated using the FLEX peptide L/H ratio as described before (see, e.g., Singh S et al., J Proteome Res. 2009, May; 8(5):2201-10). Amounts of insoluble Tau in each patient samples was calculated in the unit of fmol Tau per mg brain wet weight (average of technical replicates).

An updated version of the FLEXITau SRM assay was run for the second cohort and the frontal gyrus (BA46). After optimization of transitions using in-house DDA spectral libraries and heavy-isotope labeled Tau standards, peptide mixtures were analyzed on the same triple quadrupole mass spectrometer (5500 QTRAP, Sciex) which was coupled to an Eksigent micro-autosampler AS2 and a microflow pump (Eksigent, Dublin, CA) as described above but operated under microflow LC conditions. Here, 2 μg of peptides were loaded on a 25 cm column (Proteocol C18G 200 Å 3 μm, 250 mm length×300 μm ID, Trajan Scientific and Medical, Australia) for 3 min at 99% A (0.10% (v/v) formic acid in H₂O) and 1% B (0.1o % (v/v) formic acid in ACN) at a flow rate of 5 μl/min and separated using a 15 minutes linear gradient (1 to 70% B). The retention time window was set to 0.5s and total scan time to 0.5s, which ensured a dwell time of 10 ms of each transition pair. For the sample preparation, the FLEXITau standard was added to the sarkosyl-insoluble or soluble lysates in equimolar amounts (Light-to-Heavy (L/H) ratio 1:1) and processed as described above using FASP (FASP Protein Digestion Kit, Expedeon) and digested with 12.5 ng/μl trypsin overnight at 37° C. Vacuum-dried peptides were reconstituted in sample buffer (5% formic acid, 5% acetonitrile (ACN)) containing indexed retention time (iRT) peptides (Biognosys) and 10 fmol/μl non-labeled FLEX-peptide and 100 fmol/ul heavy-isotope labeled isoform specific peptides (AEEAGIGDTPSL[+7.1]EDEAA[+4]GHVTQA[+4]R (0N Tau), STPTAEAEEAGIGDTPS L[+7.1]EDEAA[+4]GHVTQA[+4]R (IN Tau), STPTAEDVTAPLVDEGAP[+6]GK (2N Ta u), ESPLQTPTEDGSEEP[+6]GSETSDAK (2N & IN), KLDL[+7.1]SNV[+6]QSK (4R Tau), LDL[+7.1]SNV[+6]QSK (4R Tau), VQIVYKP[+6]V[+6]DLSK (3R Tau), VPGGGSVQIVYKP[+6]V[+6]DLSK. (4R Tau) (see, e.g., Escher C et al., Proteomics. 2012 April; 12(8):1111-21). To ensure no bias in acquisition, samples were run in randomized order. SRM data were analyzed and manually validated in an updated version of Skyline (version 19.08, MacCoss Lab Software, University of Washington, Seattle, WA) (see, e.g., MacLean B, et al., Bioinformatics. 2010 Apr. 1; 26(7):966-8). All peptide transitions were evaluated for variability, similarity between y-ion ratios, dotp and dotr products, elution times, and interfering signals by manual analysis as described above. Peak boundaries were manually inspected and reassigned as needed to ensure correct peak detection and accurate integration. Peptides were quantified if the peptide transitions had a signal-to-noise of >3 and at least three light and three heavy high-quality SRM transitions were observed and normalized the same way as described above. For the isoform-specific peptides, the ratios of the heavy isotope labelled peptides were adjusted to the heavy FLEXITau standard by using the corresponding 2N4R FLEXITau peptides HVPGGGSVQIVYKPVDLSK (SEQ ID NO: 26), ESPLQTPTEDGSEEPGSETSDAK (SEQ ID NO: 27), STPTAEDVTAPLVDEGAPGK (SEQ ID NO: 28).

### LC-MS/MS Measurements and Data Analysis

Sarkosyl-insoluble samples of 29 AD patients and 28 matched control individuals were analyzed using a QExactive™ mass spectrometer (Thermo Fisher Scientific, Bremen) coupled to a micro-autosampler AS2 and a nanoflow HPLC pump (Eksigent, Dublin, CA). Peptides were separated using an in-house packed C18 analytical column (Magic C18 particles, 75 μm×15 cm; AQUA C18/3 μm, Michrom Bioresource) by a linear 120 min gradient starting from 95% buffer A (0.1% (v/v) formic acid in HPLC-H2O) and 5% buffer B (0.2% (v/v) formic acid in acetonitrile) to 35% buffer B. A full mass spectrum with resolution of 70,000 (relative to a mass-to-charge (m/z) of 200) was acquired in a mass range of 300-1500 m/z (AGC target 3×10⁶, maximum injection time 20 ms). The 10 most intense ions were selected for fragmentation via higher-energy c-trap dissociation (HCD, resolution 17,500, AGC target 2×10⁵, maximum injection time 250 ms, isolation window 1.6 m/z, normalized collision energy 27%). Due to an update in sample numbers a randomized and balanced second and third batch of 32 patient samples and 21 control samples, respectively was prepared and analyzed using a Q Exactive HF and QE mass spectrometer (Thermo Fisher Scientific, Bremen) coupled to a micro-autosampler AS2 and a nanoflow HPLC pump (Eksigent, Dublin, CA). Peptides were loaded on a capflow PicoChip column (150 μm×10 cm Acquity BEH C18 1.7 μm 130 Å, New Objective, Woburn, MA) with 2 μl/min solvent A. The proteolytic peptides were eluted from the column using 2% solvent B (0.1% FA) in solvent A, which was increase from 2% to 97% at a flowrate of 1 μl/min. The PicoChip containing an emitter for nanospray ionization was kept at 50° C. and mounted directly at the inlet to the HF mass spectrometer. The Q Exactive mass spectrometer was operated under the same mode as described above. The HF mass spectrometer was operated in positive DDA top 20 mode with the following MS1 scan settings: m/z range 350-1400, resolution 120,000@m/z 400, AGC target 3e⁶, max IT 60 ms. MS2 scan settings: resolution 60000 @m/z 400, AGC target 8e³, max IT 100 ms, isolation window m/z 1.6, NCE 27, underfill ration 1% (intensity threshold 1e⁴), charge state exclusion unassigned, 1, >6, peptide match preferred, exclude isotopes on, dynamic exclusion 40s. For a more comprehensive PTM mapping a second batch of 23 patient and control samples additionally underwent GluC and AspN digestion using the same workflow as described above. Samples were digested for 16 h with GluC followed by a 3 h digestion with AspN at 30° C. and desalted and analyzed using a Q Exactive HF mass spectrometer with the PicoChip column as described above.

For, the BA46 patient cohort, peptides were loaded onto a capflow PicoChip column (150 μm×10 cm Acquity BEH C18 1.7 μm 130 Å, New Objective, Woburn, MA) with 2 μl/min solvent A. The PicoChip containing an emitter for nanospray ionization was kept at 50° C. and mounted directly at the inlet to the QE mass spectrometer. The proteolytic peptides were eluted from the column using 2% solvent B (0.1% FA) in solvent A, which was increased from 2% to 97% at a flowrate of 1 μl/min for 60 min. The mass spectrometer was operated in positive DDA top 10 mode with the following MS1 scan settings: mass-to charge (m/z) range 350-1500, resolution 60,000@m/z 400, AGC target 3e⁶, max IT 20 ms. MS2 scan settings: resolution 17500 @m/z 400, AGC target 2e5, max IT 250 ms, isolation window m/z 1.6, NCE 27, underfill ration 1% (intensity threshold 1e4), charge state exclusion unassigned, 1, >6, peptide match preferred, exclude isotopes on, dynamic exclusion 20s.

Mass spectrometry raw data was processed using different software for the identification and quantification of post translational modification (PTM) of Tau. QExactive raw files were converted into mgf data format using ProteoWizard (see, e.g., Kessner D et al., Bioinformatics. 2008 Nov. 1; 24(21):2534-6). The spectra were centroided and filtered using ms2preproc to select the 6 most intense peaks in a 30 Th window (see, e.g., Renard B Y et al., Proteomics. 2009 November; 9(21):4978-84). Collected spectra were searched against a Homo sapiens proteome database (downloaded from uniprot.org on Nov. 1, 2017) with ProteinPilot™ Software 4.5 Beta (Paragon Algorithm 4.5.0.0. 1575, Sciex). The following settings were applied: instrument type ‘Orbi MS (1-3 ppm)’; ‘Urea denaturation’; ‘thorough’ search mode; ‘phosphorylation emphasis’, ‘acetylation emphasis’, ‘ID focus on biological modifications’. A cutoff of 85% confidence was employed for all modified peptides. In addition, all MS/MS spectra of identified post-translationally modified peptides were subjected to manual verification. Raw data were additionally analyzed by MaxQuant software version 1.6.1.10 (see, e.g., Cox J et al., Nat Biotechnol. 2008 December; 26(12):1367-72) and peptide list searched against the Homo sapiens Uniprot protein sequence database (December 2017, only reviewed entries appended with common laboratory contaminants [cRAP database, 247 entries]) using the Andromeda search engine (see, e.g., Cox J et al., J Proteome Res. 2011 Apr. 1; 10(4):1794-805). The following settings were applied: trypsin (specificity set as C-terminal to arginine and lysine) with up to two missed cleavages, mass tolerances set to 20 ppm for the first search and 4.5 ppm for the second search. Oxidation of M, acetylation of N-termini, phosphorylation of S, T, Y, acetylation of K, and ubiquitination (GlyGly) were chosen as variable modifications and propionylation of cysteine as static modification. False discovery rate (FDR) was set to 1% on peptide and protein levels with a minimum length of seven amino acids and was determined by searching a reverse database. Peptide identification was performed with an allowed initial precursor mass deviation up to 7 ppm and an allowed fragment mass deviation of 20 ppm. For all other search parameters, the default settings were used. For the Glu-C and Asp-N digested samples settings were set to the respective enzyme with up to three missed cleavages. Label-free quantification was done using the XIC-based in-built label-free quantification (LFQ) algorithm (see, e.g., Cox J et al., Mol Cell Proteomics. 2014 September; 13(9):2513-26) integrated into MaxQuant. Data analysis was performed with the Perseus software in the MaxQuant computation platform and in the R statistical computing environment.

### Preparation of High and Low Molecular Weight Tau and Mass Spectrometric Analysis

### Patient Samples

High Molecular weight Tau was prepared as described in Takeda et al., 2018 (see, e.g., Takeda S et al., Nat Commun. 2015 Oct. 13; 6:8490). Frozen brain tissues from the frontal cortex of four patients with AD and four non-demented control subjects were obtained from the Massachusetts Alzheimer's Disease Research Center Brain Bank. All the study subjects or their next of kin gave informed consent for the brain donation, and the Massachusetts General Hospital Institutional Review Board approved the study protocol. All the AD subjects fulfilled the NIA-Reagan criteria for high likelihood of AD. 300-500 mg of cortical grey matter was processed as described in the following section.

### Preparation of High and Low Molecular Weight Tau Fractions for MS

300-500 mg of frontal cortex tissue was homogenized in 5× volumes of cold Phosphate Buffered Saline (PBS), spun at 10,000 g for 10 min at 4° C. and the supernatant was used for size-exclusion chromatography. Human brain PBS-soluble extracts were separated by size-exclusion chromatography (SEC) on single Superdex200 10/300GL columns (GE Healthcare) in PBS (Sigma-Aldrich, filtered through a 0.2-μm membrane filter), at a flow rate of 0.5 ml min⁻¹, with an AKTA purifier 10 (GE Healthcare). Each brain extract was diluted with PBS to contain 6,000 ng of human Tau in a final volume of 900 μl, which was filtered through a 0.2-μm membrane filter and then loaded onto an SEC column. Fractions were collected every 1 min (0.5 ml/fraction) from 5.5 mL elution volume (Fraction 2) to 16.5 ml (Fraction 20). Fraction 3 and 4 (containing the high molecular tau) and Fraction 14, 16, 18 (low molecular tau) were collected for mass spectrometry analysis. The individual fractions separated by SEC were analyzed by ELISA (Tau (total) Human ELISA kit, diluted 1:50 in kit buffer). Four volumes (vol/vol) of cold acetone (20° C.) were added to each fraction, followed by vigorous vortex and incubation for 90 min at −20° C. Samples were then centrifuged at 13,000 g for 10 min at 4° C. After carefully aspirating the supernatant, the pellets were resuspended in 105 μl of PBS. Western Blot analysis using Tau13, Tau46, HT7 and DAKO antibodies confirmed that the fraction 3 and 4 are enriched for Tau (data not shown).

The FASP method was carried out by the method as described in e.g., Wiśniewski J R et al., Nat Methods. 2009 May; 6(5):359-62, with adjustments as described here: 100 μg proteins were denatured and reduced by adding 400 μl 8 M urea supplemented with 200 mM tris(2-carboxyethyl) phosphine (TCEP) for 30 min at 60° C. Samples were then loaded on a 10 kDa MWCO spin filter column (Milipore) and spun 14,000×g for 15 min at 23° C. Two wash steps with 200 μl 8 M urea solution were carried out and proteins were alkylated with 100 μl 0.05 M iodoacetamide solution in 8 M urea, shaking at 600 rpm for one minute and incubated in the dark at (23° C.) for 20 min before centrifugation at 14,000×g for 15 min. Two further washes with 8 M urea solution were carried out and subsequently three washes with 100 μl 50 mM ammonium bicarbonate solutions were performed. Protein digestion was performed with sequencing grade trypsin (Promega) at a nominal enzyme to substrate ratio of 1:50. After incubation for 16 h at 600 rpm, the resulting peptides were eluted in two wash steps with 50 μl ABC and a final spin with 50 μl 0.5 M sodium chloride (NaCl). Peptide eluates were acidified and desalted using reversed phase C-18 MicroSpin columns (SEMSS18R, Nest Group) and eluted in two fractions (30% Acetonitrile and 50%-70% Acetonitrile fractions). Samples were vacuum dried and frozen at −20° C. prior to LC-MS/MS analysis.

### Lc-MS/MS Analysis of High and Low Weight Molecular Tau

Randomized samples (SEC fractions 2, 3, 4 and 14, 16, 18 for 4 AD and 4 control human brain samples) were analyzed in duplicates using a Q Exactive™ mass spectrometer (Thermo) coupled to a micro-autosampler AS2 and a nanoflow HPLC pump (Eksigent). Peptides were separated using an in-house packed C18 analytical column (Magic C18 particles, 75 μm×15 cm; AQUA C18/3 μm, Michrom Bioresource) by a linear 120 min gradient starting from 95% buffer A (0.1% (v/v) formic acid in HPLC-H2O) and 5% buffer B (0.2% (v/v) formic acid in acetonitrile) to 35% buffer B. A full mass spectrum with resolution of 70,000 (relative to an m/z of 200) was acquired in a mass range of 300-1500 m/z (AGC target 3×10⁶, maximum injection time 20 ms). The 10 most intense ions were selected for fragmentation via higher-energy c-trap dissociation (HCD, resolution 17,500, AGC target 2×10⁵, maximum injection time 250 ms, isolation window 1.6 m/z, normalized collision energy 27%). Q Exactive raw files were converted into mgf data format using ProteoWizard (Kessner, Darren, et al. “ProteoWizard: open source software for rapid proteomics tools development.” Bioinformatics 24.21 (2008): 2534-2536). The spectra were centroided and filtered using ms2preproc to select the 6 most intense peaks in a 30 Th window (De Souza Jr et al., “Control of a chaotic polymerization reactor: A neural network based model predictive approach.” Polymer Engineering & Science 36.4 (1996): 448-457). Collected spectra were searched against a Homo sapiens database (uniprot.org on Apr. 2, 2016) with ProteinPilot™ Software 4.5 Beta (Paragon Algorithm 4.5.0.0. 1575, Sciex). The following settings were applied: instrument type ‘Orbi MS (1-3 ppm)’; ‘Urea denaturation’; ‘thorough’ search mode; ‘phosphorylation emphasis’, ‘acetylation emphasis’, ‘ID focus on biological modifications’. A cutoff of 99% confidence was employed for all modified peptides. In addition, all MS/MS spectra of identified post-translationally modified peptides were subjected to manual verification. Raw data were analyzed by MaxQuant software version 1.6.1.10 (see, e.g., Cox J et al., Nat Biotechnol. 2008 December; 26(12):1367-72) and peptide list searched against the Uniprot protein sequence database (February 2016, only reviewed entries appended with common laboratory contaminants [cRAP database, 247 entries]) using the Andromeda search engine (see, e.g., Cox J et al., J Proteome Res. 2011 Apr. 1; 10(4):1794-805). The following settings were applied: trypsin (specificity set as C-terminal to arginine and lysine) with up to two missed cleavages, mass tolerances set to 20 ppm for the first search and 4.5 ppm for the second search. Oxidation of M, N-terminal acetylation, phosphorylation of STY, Ubiquitination (GlyGly) and acetylation of K were chosen as dynamic modifications and carbamidomethylation of cysteine as static modification. False discovery rate (FDR) was set to 1% on peptide and protein levels with a minimum length of seven amino acids and was determined by searching a reverse database. Peptide identification was performed with an allowed initial precursor mass deviation up to 7 ppm and an allowed fragment mass deviation of 20 ppm. For all other search parameters, the default settings were used.

### Preparation and Mass Spectrometry Analysis of MCI-Isolated Tau

MC1-isolated Tau was obtained from Peter Davis, purified from lysate cleared of large particles and aggregates of 4 separate AD patients using MC-1 antibody immunoaffinity columns (see, e.g., Jicha G A et al., Journal or Neurochemistry 72(1):214-24). Purified PHF Tau was tryptically digested using the FASP method as described above, with the addition of heavy Tau standard peptide spiked in for FLEXITau experiments. Peptide elutes were acidified and desalted using reversed phase C-18 microspin columns (SEMSS18R, Nest Group), vacuum dried and frozen at −20° C. prior to LC-MS/MS analysis. For data-dependent acquisition experiments LC-MS/MS analysis, peptides were loaded on a capflow PicoChip column (150 μm×10 cm Acquity BEH C18 1.7 μm 130 Å, New Objective, Woburn, MA) with 2 μl/min solvent A. The proteolytic peptides were eluted from the column using a 60 min gradient starting at 2% solvent B (0.1% FA) in solvent A, which was increased to 35% at a flowrate of 1 μl/min. The PicoChip containing an emitter for nanospray ionization, which was kept at 50° C. and mounted directly at the inlet to the HF mass spectrometer. The mass spectrometer was operated in positive DDA top 20 mode with the following MS1 scan settings: mass-to charge (m/z) range 300-1650, resolution 60,000@m/z 400, AGC target 3e⁶, max IT 20 ms. MS2 scan settings: resolution 30000 @m/z 400, AGC target 1e⁵, max IT 25 ms, isolation window m/z 1.4, NCE 27, charge state exclusion unassigned, 1, >8, peptide match preferred, exclude isotopes on, and dynamic exclusion of 20s. Raw data were analyzed by MaxQuant software version 1.6.2.1 and peptide list searched against the Homo sapiens Uniprot protein sequence database including isoforms (February 2016, only reviewed entries appended with common laboratory contaminants [cRAP database, 247 entries]) using the Andromeda search engine. The following settings were applied: trypsin (specificity set as C-terminal to arginine and lysine) with up to two missed cleavages, mass tolerances set to 20 ppm for the first search and 4.5 ppm for the second search. Oxidation of M, acetylation of N-termini, phosphorylation of STY, ubiquitination (GlyGly) and acetylation at K, were set as variable modifications and propionylation of cysteine as static modification. False discovery rate (FDR) was set to 1% on peptide and protein level and was determined by searching a reverse database. Peptide identification was performed with an allowed initial precursor mass deviation up to 7 ppm and an allowed fragment mass deviation of 20 ppm. For all other search parameters, the default settings were used. Label-free quantification was done using the XIC-based in-built label-free quantification (LFQ) algorithm integrated into MaxQuant. Spectra for identified modified peptides were manually validated. For FLEXITtau SRM experiments, flex-peptide was spiked in to PHF digests (final concentration of 0.05 pmol/uL and targeted LC-MS/MS was performed using the microflow FLEXITau method as outlined above for the Tau sarkosyl fractions.

### Statistical Data Analysis

Data was tidied up using tidyverse (1.3.1), dyplr (0.8.5), reshape2 (1.4.2), FactoMinR (2.1), plyr (1.8.6) and Microsoft Excel. Statistical data analysis was carried out using a combination of Prism 8.132, Perseus, and R programming language (3.2.1, 3.4, 3.5.1, and 3.6.0). In R several packages were used. GGplot2 (3.1.1) and pcaMethods (1.78.0) were used for principal component analysis plots, OPLS-DA plots were generated using ropls (1.18.1); pheatmap (1.0.8), heatmaply (1.0), d3heatmap (0.6.1.2) for plotting heatmaps and data exploration, FactorMineR (2.1) to calculate PCA confidence ellipses and plot colors were generated using RColorBrewer (1.1.-2) for initial color palettes. The package gradDescentR (1.1.1) was used for building the classifier using stochastic gradient descent. The shinyBS (R package version 0.20) was employed for initial visualizations. Plot colors and plot layout and sizes were finalized using inkscape. For Hierarchical clustering—Euclidean distance was used with a complete clustering of rows.

### Example 2: Tau Concentrations and Isoform Distributions in Alzheimer's Disease (AD) and Control Subjects

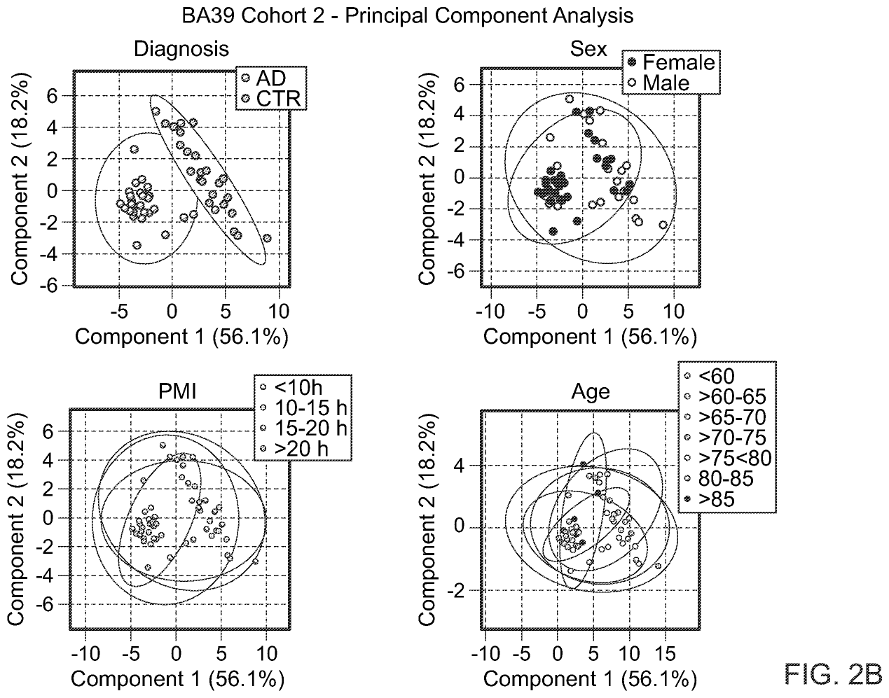

Multiple types of qualitative and quantitative mass spectrometry analyses were employed to characterize the molecular features of Tau from the BA39 angular gyrus region in post-mortem brain tissue from two cohorts with overlapping subjects comprising a total of 49 AD patients and 42 age-, PMI-, and sex-matched control subjects. The subjects were selected based on the criteria that they were either diagnosed prior to death as clinically AD or were asymptomatic (control subjects). The next criteria was definitive post-mortem neuropathological final diagnosis by the respective brain banks as either AD or unaffected. Selected subjects had the least possible concurrent pathologies and were assessed for Braak (Braak, H., and Braak, E. (1991). Neuropathological stageing of Alzheimer-related changes. Acta Neuropathol 82, 239-259) Neurofibrillary tangles (NFTs) stages as determined by the location of NFTs with a total Tau immunostaining and Bielschowsky's silver stain. Inclusion in the study required a high confidence post-mortem neuropathological diagnosis of AD for the clinical AD group or no more than low AD Neuropathologic Change (Montine et al. (2016). Multisite assessment of NIA-AA guidelines for the neuropathologic evaluation of Alzheimer's disease. Alzheimers Dement 12, 164-169) or a Braak Stage less than IV for the clinical control group. Principal Component Analysis (PCA) analysis did not show any effect of age, PMI, and sex on the Tau PTM profiles for both cohorts (FIGS. 2A and 2B). The angular gyrus was selected as a brain region of intermediate pathology in the end stage of disease without a large amount of neuronal loss. To determine if Tau PTMs and quantitative profiles were observed in other affected brain regions, we analyzed the molecular features of Tau from the BA46 region in 9 control and 10 AD patients using complementary analysis. A schematic overview of the cohort selection, sample preparation and mass spectrometry analysis are shown in FIG. 3A.

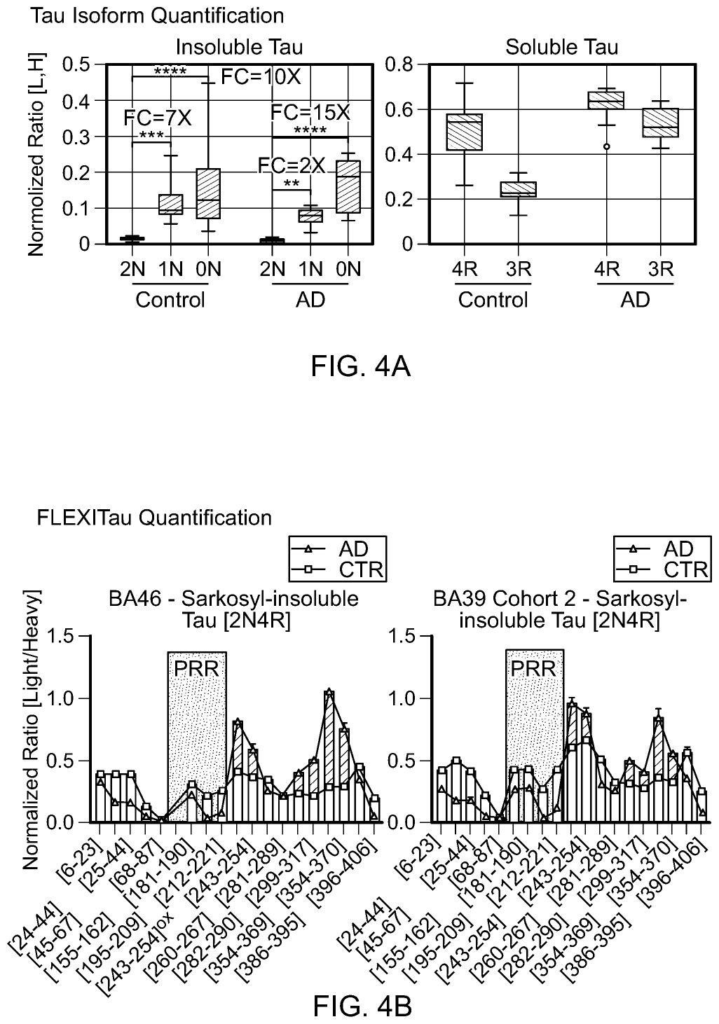

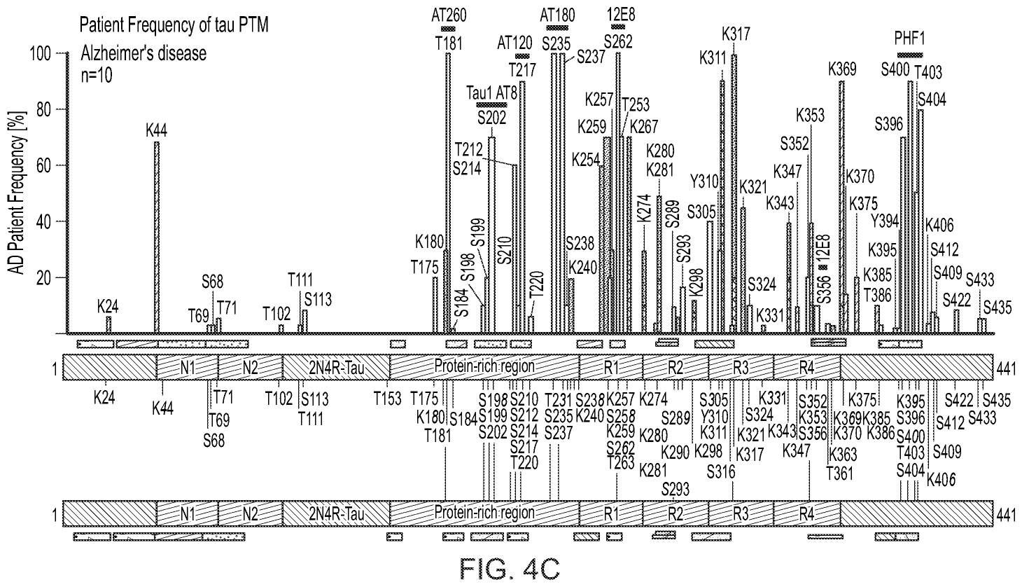

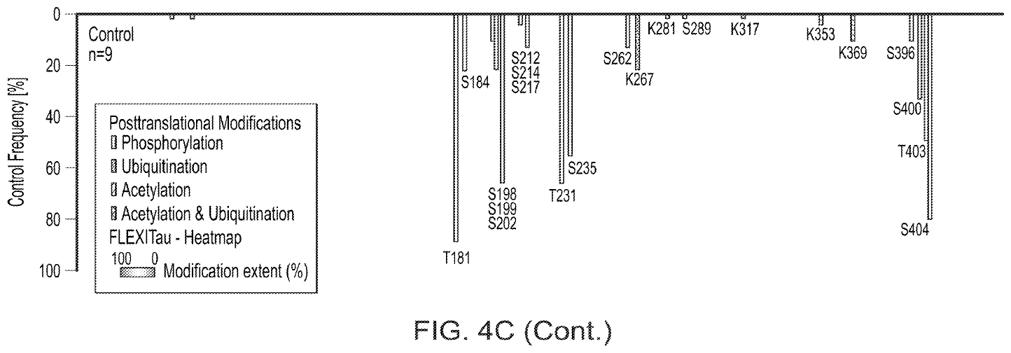

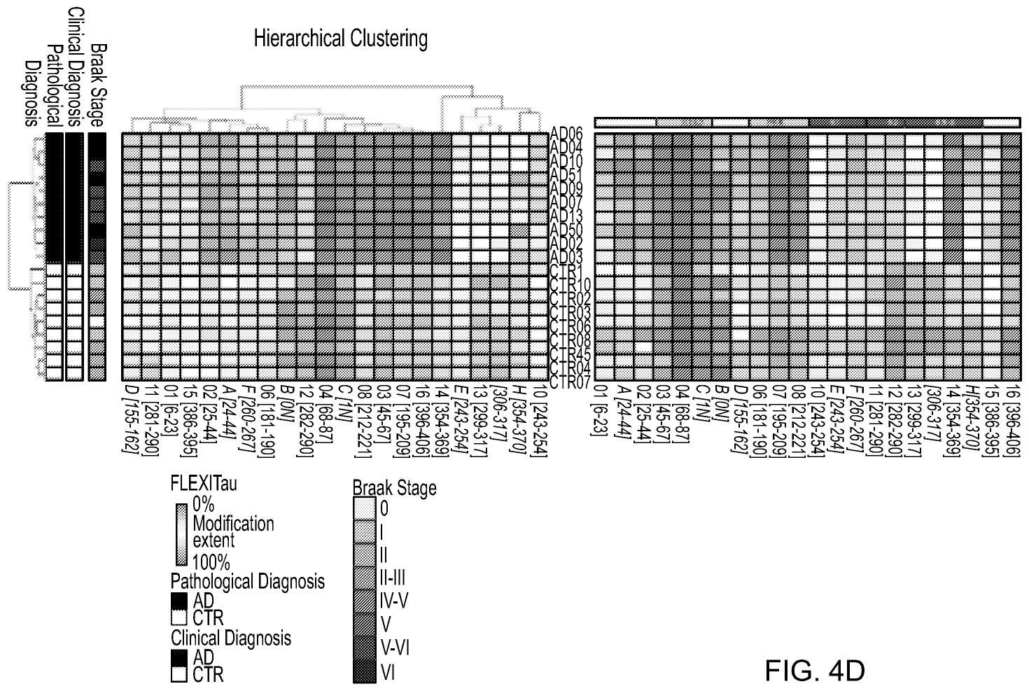

FLEXITau quantification method was employed to determine the molar concentration (absolute quantification) of Tau from sarkosyl soluble and insoluble fractions of cortical grey matter from parietal lobe association cortex (BA39). Total amount of soluble Tau is lower in AD patients (˜3000 fmol/mg fresh weight of tissue) compared to the control subjects (˜5000 fmol/mg fresh weight of tissue) (FIG. 3B). The median concentrations for the pathological insoluble Tau in BA39 region are 100-fold higher in AD (˜1000 fmol/mg fresh weight of tissue) compared to controls (˜10 fmol/mg fresh weight of tissue) (FIG. 3C). It is important to note that insoluble Tau also accumulates in the age-matched control subjects albeit at lower concentrations. FLEXITau quantified a total of 24 Tau peptides, of these some were isoform specific, achieving a total sequence coverage of 61% for the 2N4R Tau isoform (FIG. 3D). Relative quantities of the 0N, TN, 2N, 3R and 4R Tau isoform specific peptides were measured in both the soluble and insoluble fractions (FIG. 3E). Interestingly, in soluble Tau, the most abundant species are the TN and 4R Tau isoforms, whereas in the insoluble fraction containing pathologic Tau, 0N and TN Tau significantly enriched compared to the 2N isoform, and the 4R is significantly enriched compared to the 3R isoforms. The absolute quantification of Tau isoforms shows that the distribution of different isoforms in the soluble fractions are not mirrored in the pathological insoluble fraction that contains the fibrils. Instead, the TN isoform is significantly depleted and the 0N isoform is enriched in the insoluble fraction. These data demonstrate that the 0N and 4R isoforms are more prone to aggregation in AD and findings were also recapitulated in sarkosyl insoluble Tau extracts from frontal gyrus (BA46) brain tissue (FIG. 4A). The data and analyses shown in FIGS. 4A-4D demonstrate that that the highest frequency PTMs, isoform distributions and the quantitative profiles of the BA46 region are shared between the two brain regions.

### Example 3: Qualitative and Quantitative Maps of Tau PTMs and their Frequency in AD Patient

Qualitative and quantitative analysis of all Tau isoforms in two different cohorts and two different brain regions, identified a) the types of PTM, b) the PTM localization c) the PTM subject frequency and d) the PTM stoichiometry based on the FLEXITau profile. We identified a total of 95 distinct modifications on 88 amino acid residues, with a cumulative sequence coverage of 96% from detected modified and unmodified 2N4R Tau peptides (FIG. 3E). While 86 phosphorylations have been described on Tau in general (Arendt et al., 2016), i.e. in-vivo, in-vitro and across different organisms and model systems, and over 31 phosphorylation sites have been identified to be associated with physiological functions (Hasegawa et al., 1992; Lovestone and Reynolds, 1997; Morishima-Kawashima et al., 1995), only 45 Tau phosphorylations have been described on pathological human Tau so far, that is, Tau analyzed from primary human specimens. Our analysis extended this to 55 phosphorylation sites on pathological insoluble Tau in BA39 and 43 phospho-sites in B46 (FIG. 4C). A total of 28 ubiquitination sites were previously identified on Tau extracted from human post-mortem AD brain total lysates using ubiquitination enrichment. In this study, we show that 17 ubiquitin sites are specific to pathological Tau (insoluble Tau) (14 in BA46, FIG. 4C). Sixteen out of 17 identified ubiquitination sites are within the Microtubule Binding Domain (MBD), which forms the core of the filament. Nineteen distinct acetylation sites were mapped in BA39 and 18 in BA46. Interestingly, some of these acetylation sites are also ubiquitinated in pathological Tau. Four methylation sites were also detected.

The patient frequencies of these 95 PTMs are shown in FIG. 3F (for BA46 see FIG. 4C), where we compare insoluble Tau from AD vs. control subjects. The frequency is key to understanding the pathology of the different PTMs and the heterogeneity of the patient population. Some of the 88 sites of modification mapped are modified by a single type of PTM, however others can carry multiple types of modification such as K311 can either be ubiquitinated or acetylated. There are multiple sites that are observed in one or two patients in both the cohorts and as such are less likely to be relevant to disease such as phosphorylation at S113. Heatmaps of median peptide modification extent from the FLEXITau MS data (Cohort 1 and Cohort 2) are overlaid with the frequency data to evaluate the stoichiometry of the PTM. The PTMs previously known to be significant and correlated with disease such as the AT8 epitope containing the phosphorylation sites S199, S202 and T205 are found with high frequency and high stoichiometry in the patient population, given this the most relevant modifications are the ones that show high frequency and extent of modification in the AD subjects.

A high frequency of acetylation occurs in the R4 region including the sites K353, K369, K370, and K375 while ubiquitination occurs in the R1-R3. This is also reflected in the frontal gyrus (BA46) (FIG. 4C). Interestingly, within R3, the hexapeptide VQIVYK at site K311 shows both acetylation and ubiquitination, with increased propensity for ubiquitination. In FIG. 3F, the FLEXITau data shown underneath the frequency graph displays the extent of modification of the PTMs for 49 AD and 42 control patients and aligns with the PTM frequencies.

Phosphorylation sites cluster with highest frequency in the Proline Rich Region (PRR). In particular, the peptides spanning [195-209] and [212-224] that encompass the sites (S198, S199, S202, T205) and (S212, S214, S217), respectively are observed to be >90% modified in AD in the FLEXITau data. In addition, the region spanning amino acid residues [396-406]features high frequency phosphorylation at S396, S400, T403, and at S404 and show >90% modification extent (FIG. 3F). This observation is also true for BA46 (FIG. 4C). Interestingly, for both brain regions (FIGS. 3F and 4C), there are some high frequency modifications in the insoluble Tau from controls such as T181 and T231. In some control subjects, these phosphorylated peptides are further phosphorylated and show some of the same modifications associated with pathological Tau. These phosphorylation sites at S198, S199, S202, T205 in the peptide [195-209] are observed in 20-40% of the controls, suggesting that early disease is observed in some control subjects.

### Example 4: Individual Tau PTM Profiles Shows Heterogeneity and Reflect Disease Progression

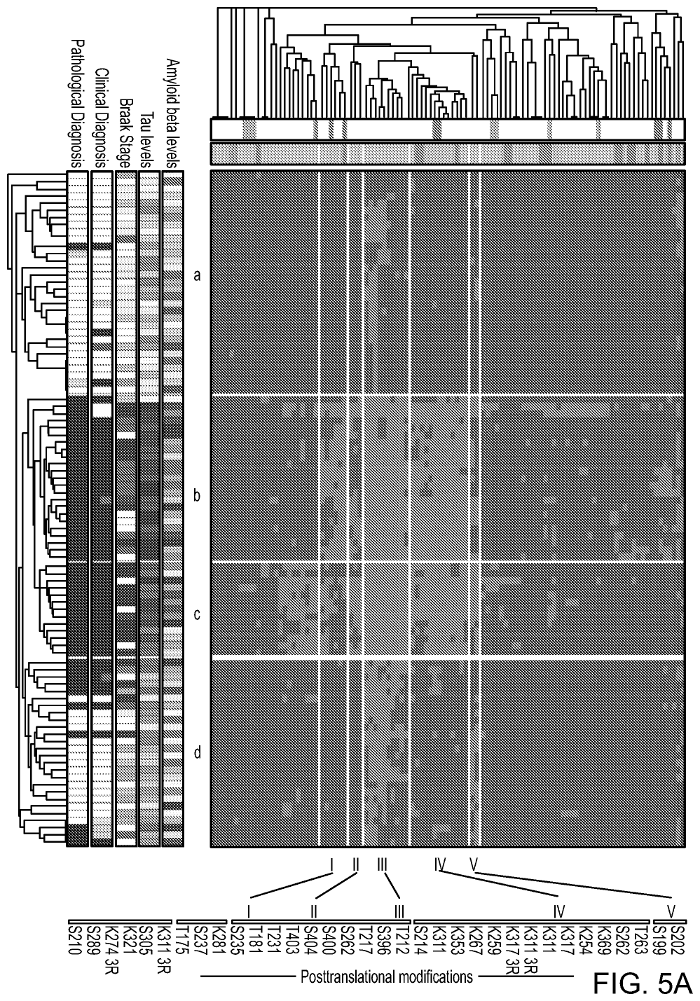

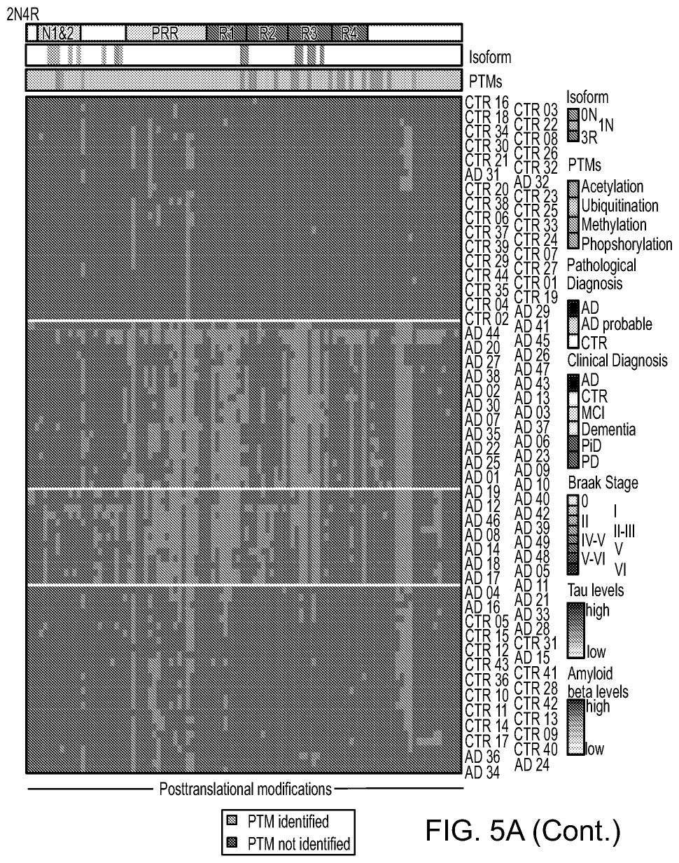

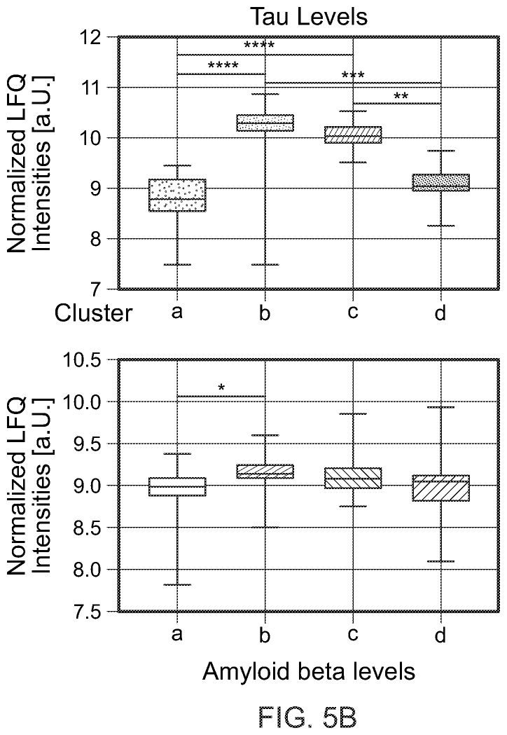

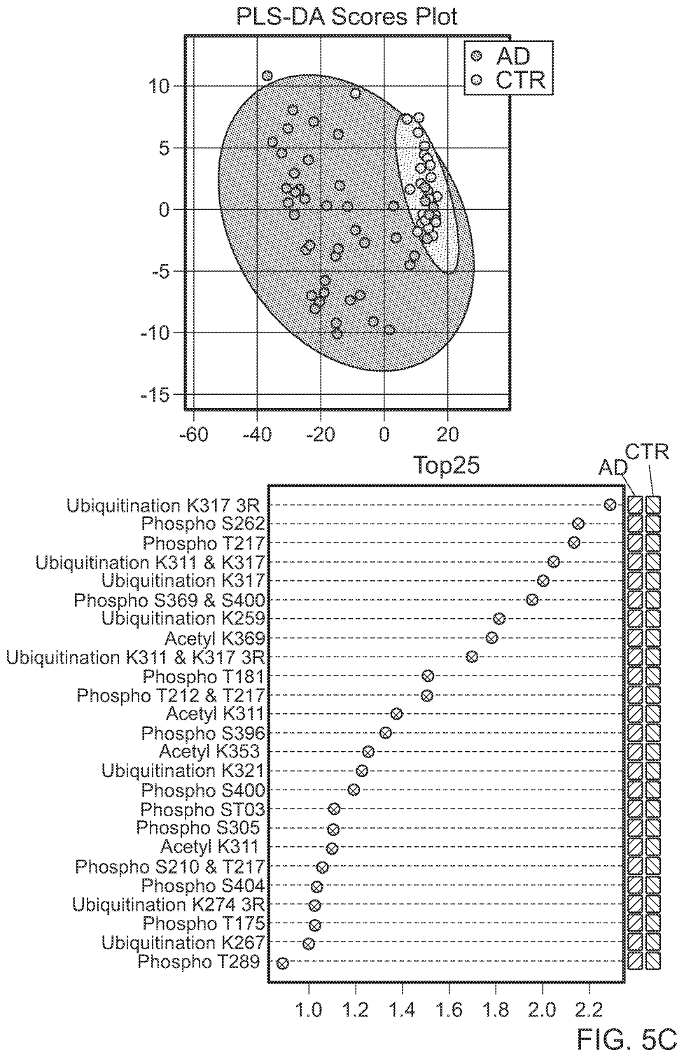

FIG. 5A depicts the unsupervised hierarchical clustering analyses of Tau PTMs identified in AD and control subjects across the two cohorts. The left panel shows the PTMs as clustered by PTM features whereas the right panel shows the PTM features arranged from N-terminal to C-terminal of Tau. Information about clinical and pathological diagnosis as well as Braak and Braak (Braak) staging are provided on the left of the figure for subjects where the data were available (23 AD patients and 27 control subjects had Braak staging data). In addition, the levels of beta amyloid and Tau measured in each sample are shown (see pink and blue heatmap). ANOVA analysis showed higher Tau and beta amyloid levels in clusters with higher Braak stage and increased level of Tau PTMs (FIG. 5B). A large percentage of AD patients cluster into two major groups (FIG. 5A, b and c), whereas the controls fall into two clusters (FIG. 5A, a and d).

The control cluster a has the lowest number of PTMs and we observe the peptide [225-242] with single phosphorylation at (T231 or S235) and some subjects with phosphorylation at T181. This cluster a is largely comprised of patients staged at Braak 0-III. Cluster d differs from a by the occurrence of phosphorylation at epitopes identified by the AT8 antibody (d-V: S199 and S202); the PHF1 antibody (d-III: S396, S400, T403 and S404); the AT100 antibody (d-III; T212 and T217); and the single S262 phosphorylation. Tau from cluster a control subjects display phosphorylation sites that are associated with normal physiological function, whereas control subjects in cluster d show additional sites associated with pathology. The majority of subjects in d are listed as control subjects and these modifications are likely to be the earliest signs of pathology. Cluster d contains 10 symptomatic patients with late stage AD pathology Braak IV-VI and 16 asymptomatic controls at Braak 0-II. The 10 symptomatic patients show some ubiquitination in the MBD domain and this may be the defining feature for symptomatic patients. The 16 asymptomatic patients display the increased phosphorylation of the PRR domain such as T181, which has been reported to be increased in ante-mortem plasma of Mild Cognitive Impairment (MCI) (Janelidze et al., 2020), (Thijssen et al., 2020). The increased phosphorylation of the PRR domain is also observed in the symptomatic patients in cluster d as well as in the AD clusters b and c, thus, although patients were not yet diagnosed symptomatic, they may have shown early cognitive defects at the time of death. The majority of AD patients separate into cluster b and c and a minority cluster with the control cluster d which show some early pathology.

The mode of Braak stage of the cluster b is Braak-V, whereas the mode Braak stage of cluster c is Braak-VI and subjects in these clusters have significantly higher Tau and amyloid burden in BA39 than subjects falling into cluster a and d (FIG. 5B). The hallmark of cluster b and c are the modifications in the MBD domain labelled as IV. These PTMS include phosphorylations occurring at adjacent sites (S262, S263), acetylations (K311, K353, K369), and ubiquitinations (K259, K267, K311, K317). These modifications are not identified in any of the other clusters. Phosphorylation sites previously identified in cluster d in the PRR (T231, S235, S212, S217) and the PHF1 epitope (S396, S400, T403, S404) are highly populated in cluster b and c. Cluster b and c display many of the same modifications, however, in cluster c we observe a higher frequency of several PTMs including T175, S237, K281 (and S191, T205 and S113 which are difficult to observe at lower stoichiometry, particularly as the S113 is a phosphorylation site found on a 0N Tau peptide). The higher occupancy in c II PTMs at T175, S237 and K281 are associated with late stage disease (Braak VI). These analyses show that subjects free of AD pathology accumulate some insoluble Tau without the hallmark AD modifications and that some clinically asymptomatic patients display Tau PTMs associated with pathology.

We used PLS-DA (Partial Least Square Discriminant Analysis) to determine which of the robustly quantifiable post translationally modified peptides are most important for the separation of the AD and control groups (FIG. 5C). The analysis provides a VIP score that provides a measure for the importance of each modified peptide in differentiating between AD and control groups. This ranks the singly and doubly ubiquitinated peptides at residue K311 and K317 as well as two phosphorylated peptides (T217 and S262) as the most distinct in differentiating between AD and control groups using quantitative data from post translationally modified peptides.

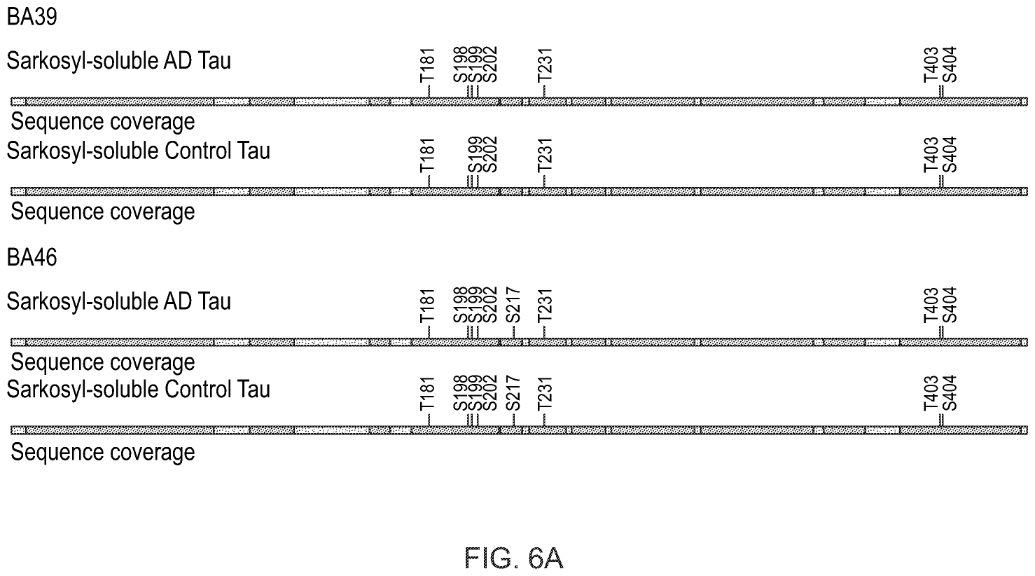

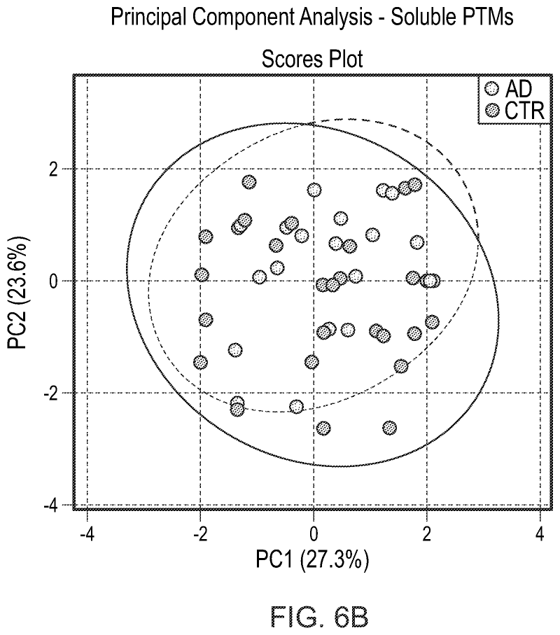

Interestingly, the soluble fraction of Tau was also studied using both qualitative and quantitative mass spectrometry and the PTM maps from AD and control subjects show no differences with respect to the identity and the quantity of the detected modifications in the soluble fractions (FIGS. 6A and 6B). This is in agreement with the suggestion that the modifications associated with the soluble fraction are associated with normal physiological Tau functions. While the top ranked PTMs identified by the PLS-DA analysis in the insoluble fraction including the ubiquitinated peptides at residue K311 and K317 may be possible biomarkers for diagnosis, an appropriately powered study in relevant specimen is required for validation.

### Example 5: Tau Peptide Stoichiometry Data Identifies Key Pathological Features in Disease

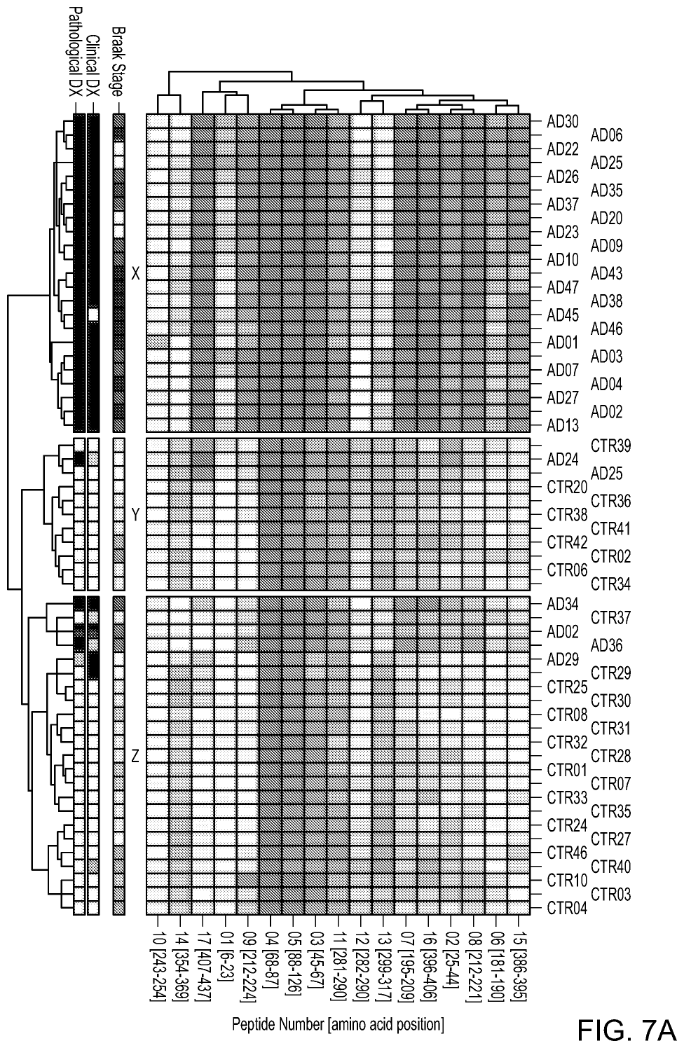

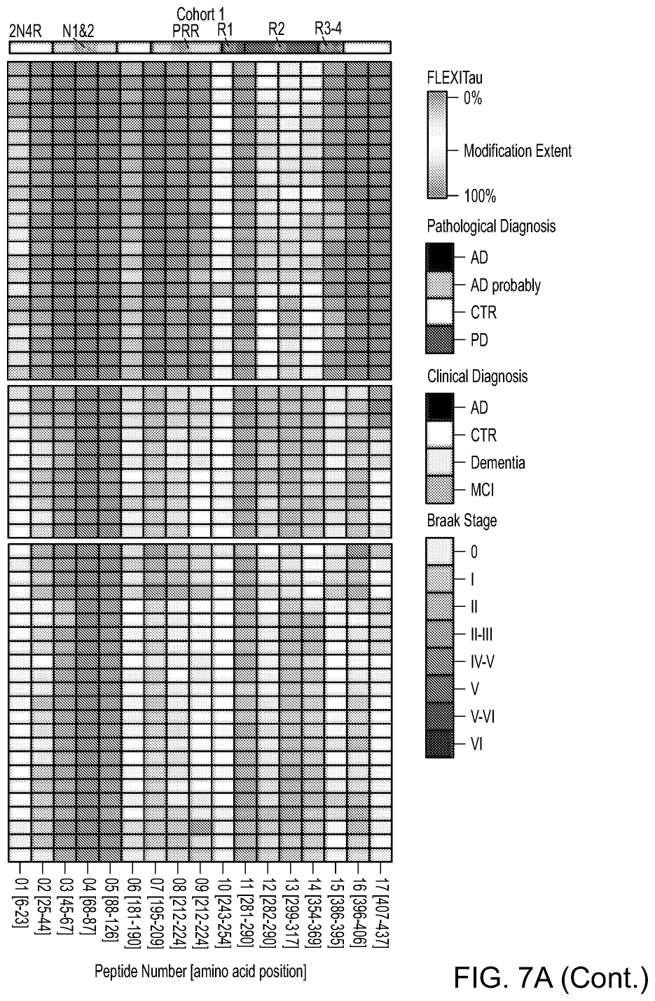

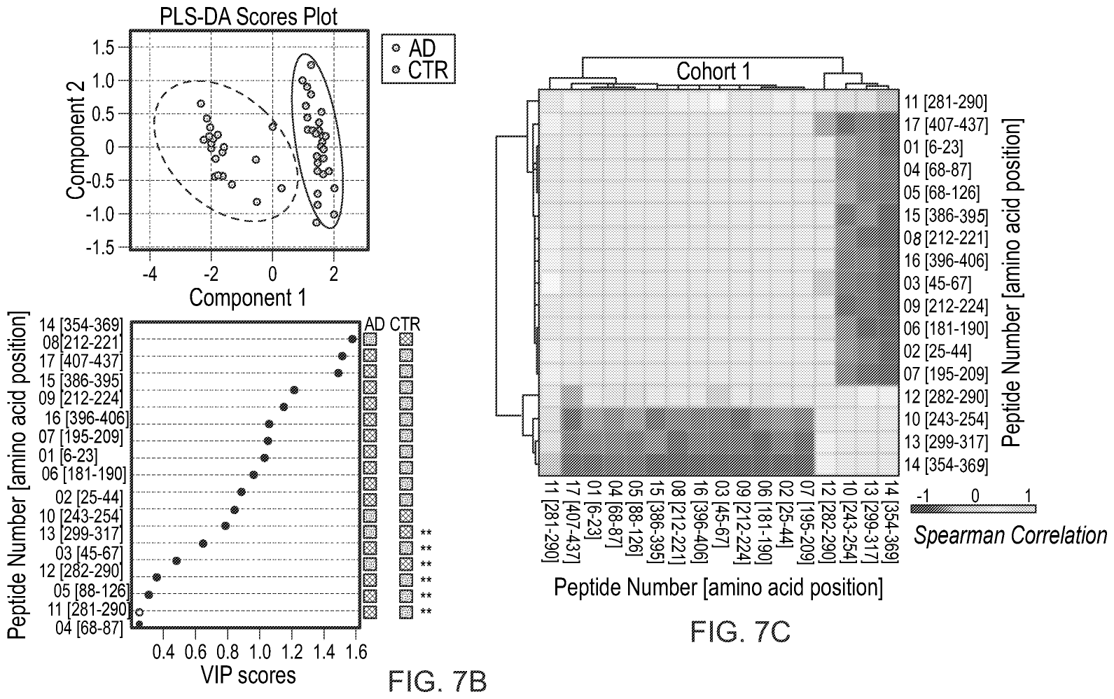

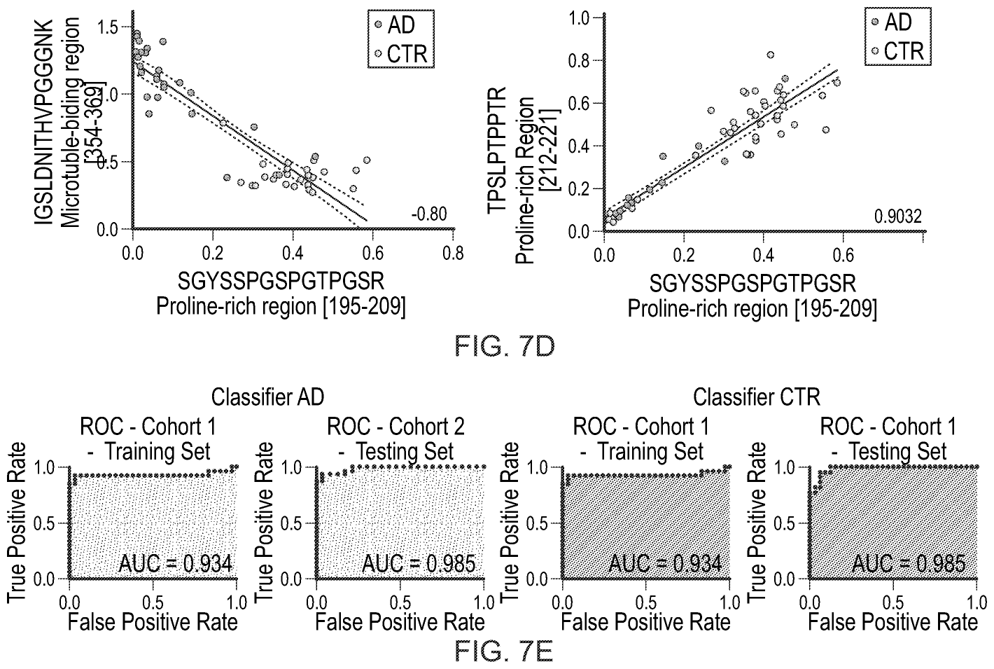

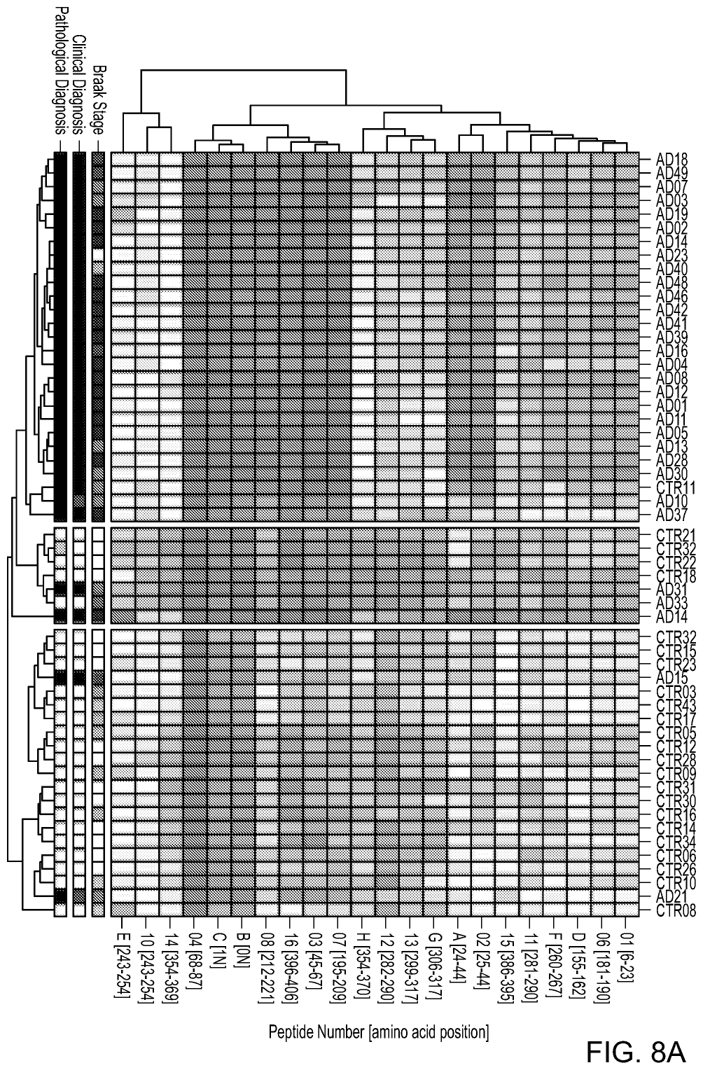

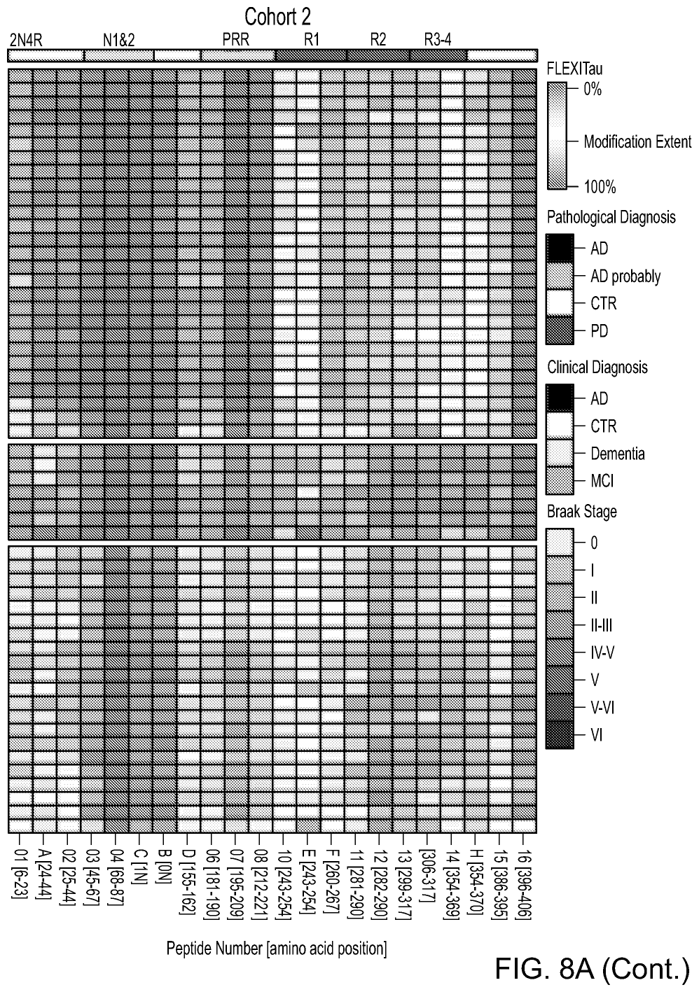

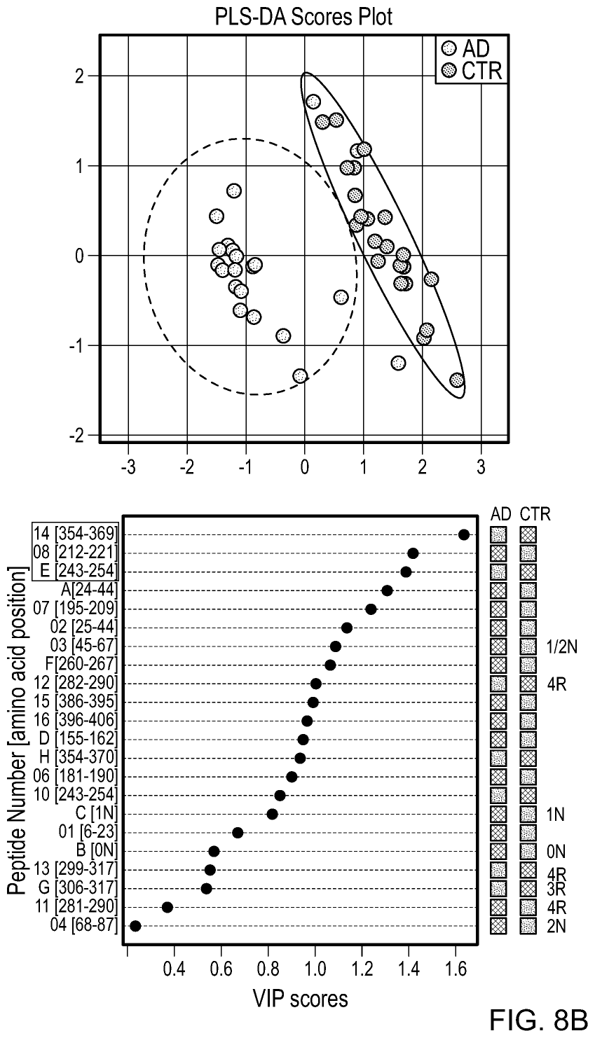

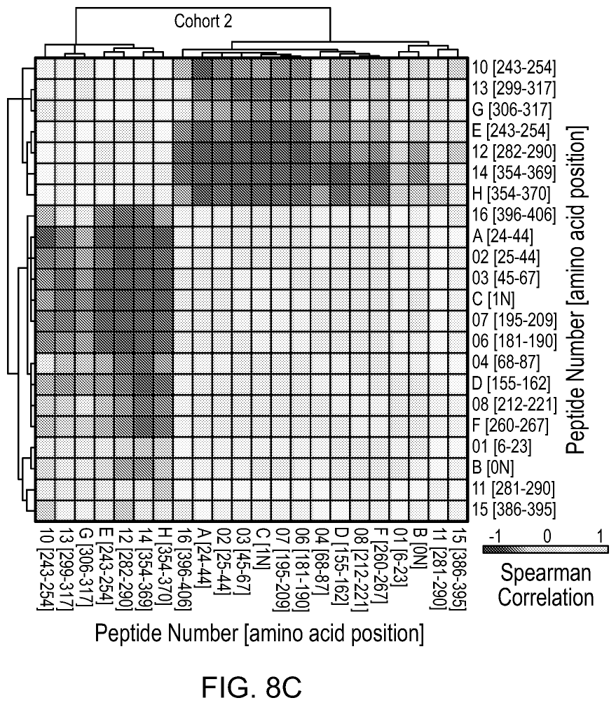

The FLEXITau data for individual patients of the two cohorts are presented in FIGS. 7A-E (FIG. 8 for cohort 2, FIG. 4D for BA46). Hierarchical cluster analysis of FLEXITau data are shown as heatmaps with the highest extent of modification shown in red and the lowest in blue. On the left, we show the hierarchical clustering of peptides showing the peptides that distinguish between clusters and the extent of modification is shown from N- to C-terminus of Tau on the right. The heatmap of the Tau PTM modification stoichiometry in AD is so distinct from control patients that they separate at the first branch of the hierarchical clusters. One defining feature for AD is the high extent of modification in the PRR spanning from amino acid (aa) 195-224 [peptides 195-209; 212-221/224]. In addition the R1-R4 region from aa 243-370 is highly enriched in the AD patients [243-254; 260-267; 281/282-290; 299-317; 306-317; 354-369/370](FIG. 7A and FIG. 8) and most of the peptides within this region are highly modified R2-R3 [260-267; 281-290; 282-290; 299-317; 306-317]. The N specific region is largely underrepresented [45-67; 68-87; 88-126] as is the C-terminus in AD. This signature of an enrichment of MBR peptides, a high modification extent in the PRR and an underrepresentation of the N-specific regions and C-terminus was also replicated in cohort 2 (FIG. 8A) and in the BA46 FLEXITau cluster analysis (FIG. 4D). We employed PLS-DA to study which variables are most important for the separation of the AD and control groups (FIG. 7B, FIG. 8B). The analysis provides a VIP score that provides a measure for the importance of each peptide in differentiating between AD and control groups (FIG. 7B, FIG. 8B). This ranks the R4 peptides 14 [354-369], the PPR peptide 8 [212-221]harboring S212, S214, S217 and T220 phosphorylation sites and the C terminal peptide 17 [407-437] as the most distinct in differentiating between AD and control groups (FIG. 7B).

A Spearman correlation analysis shows that the PRR and the C-terminal peptides, are anti-correlated with an increase in abundance of the MBD unmodified peptides in both cohorts (FIG. 7C, 7D and FIG. 8C) because both the C-terminal and the PRR regions show a high modification stoichiometry as reflected by the decrease in abundance of the unmodified peptides. This increase in phosphorylation of Tau may be involved in neutralizing charge in the MBD domain to promote aggregation and one could invoke a variant of the hairpin model to support this idea (Jeganathan et al., 2006). Given this relationship the PRR and C-terminal peptide intensities correlate with each other (FIG. 7C, 7D and FIG. 8C).

A closer examination shows heterogeneity in modification stoichiometry within the AD patients and within controls. While the unmodified C-terminal peptide is found in low abundance in AD compared to all controls, there is a control cluster where the C-terminus is completely unmodified (FIG. 7A), whereas the other control cluster shows some processing. Apart from this C-terminal peptide, all controls seem to show the same general pattern of peptide stoichiometries, for example the exon 1 peptide [6-23]shows less modification extent than all other AD patients and the PRR is somewhat modified in all controls. This suggests that the modification at the C-terminal is an early event during aging and AD pathology and is most likely attributed to the caspase 3 and 6 cleavage at D418 and D421.

Finally, we trained a classifier using stochastic gradient descent (SGD) on the FLEXITau data of cohort 1 which we tested in cohort 2. This classifier was able to predict diagnostic pathology with an accuracy of >0.92 in both cohorts (FIG. 7E).

### Example 6: Identifying PTMs Associated with Seeding Activity in Size Fractionated Tau

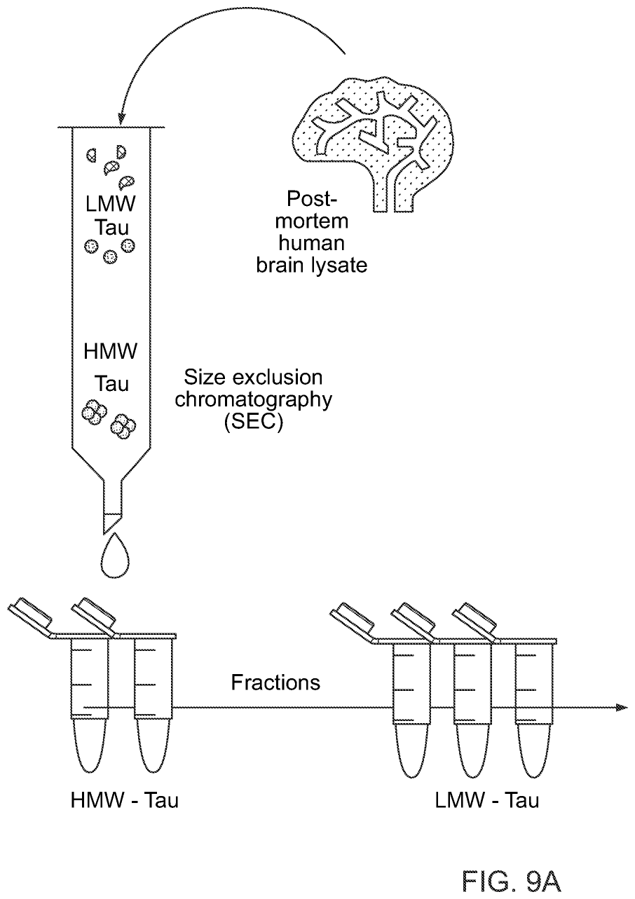

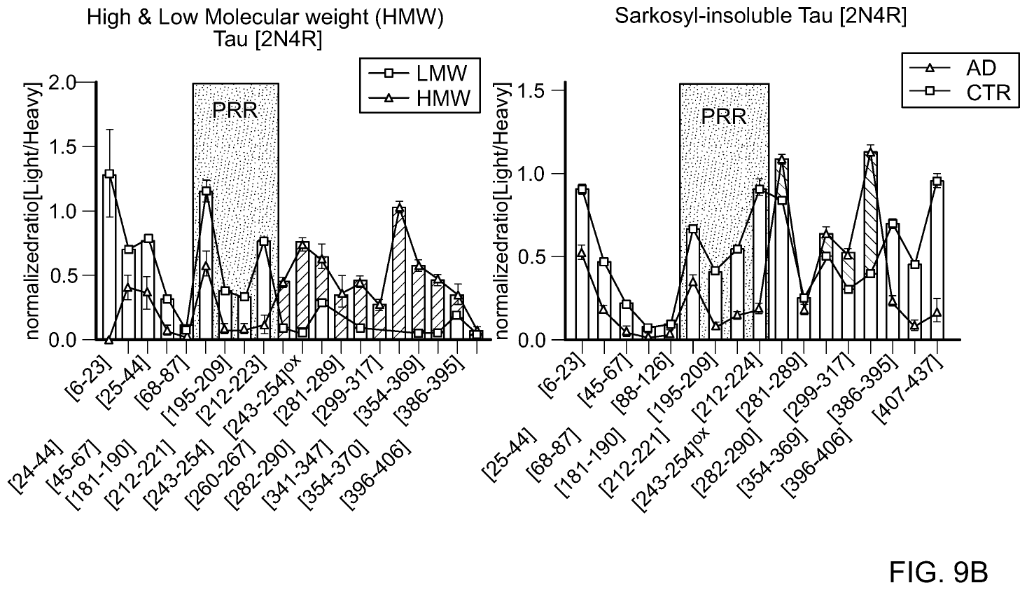

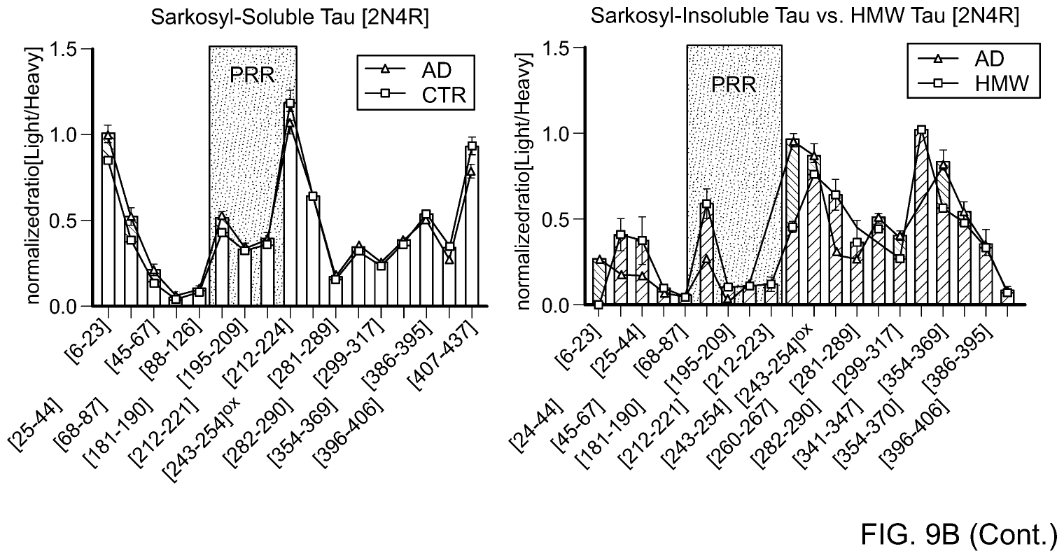

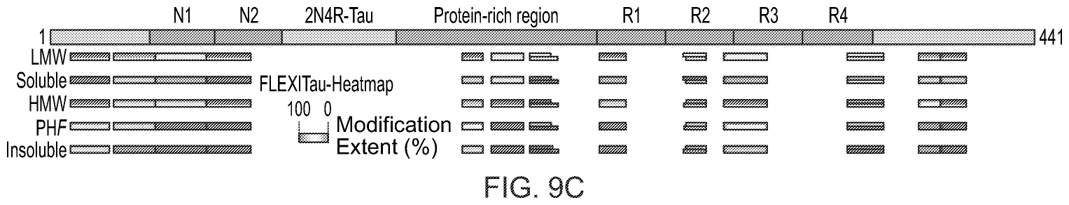

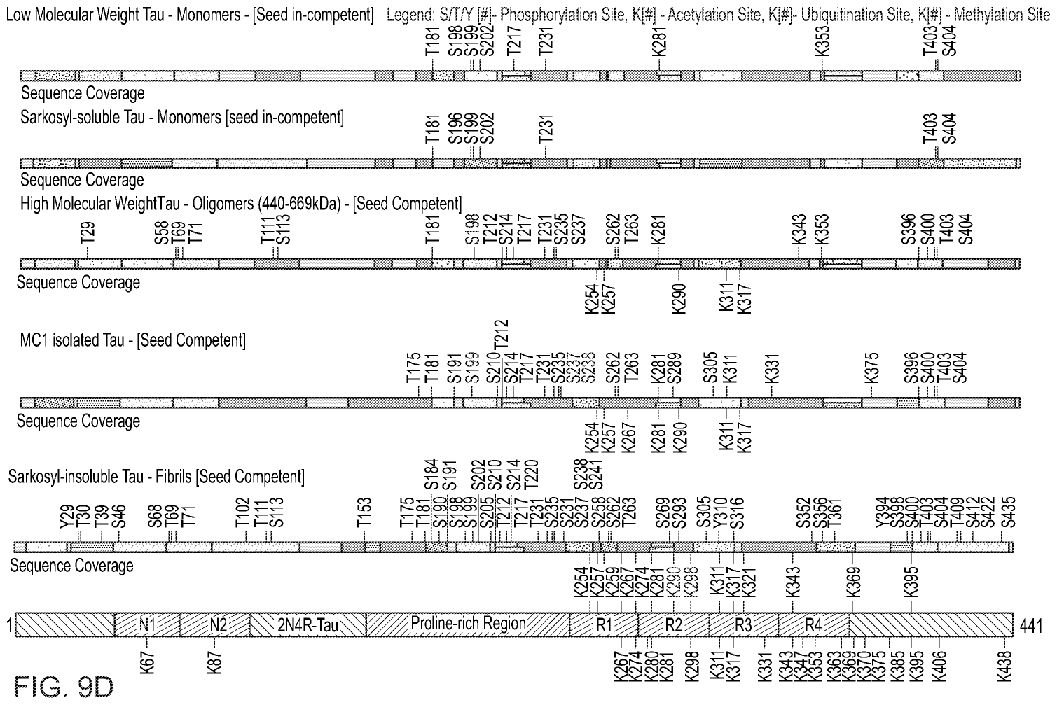

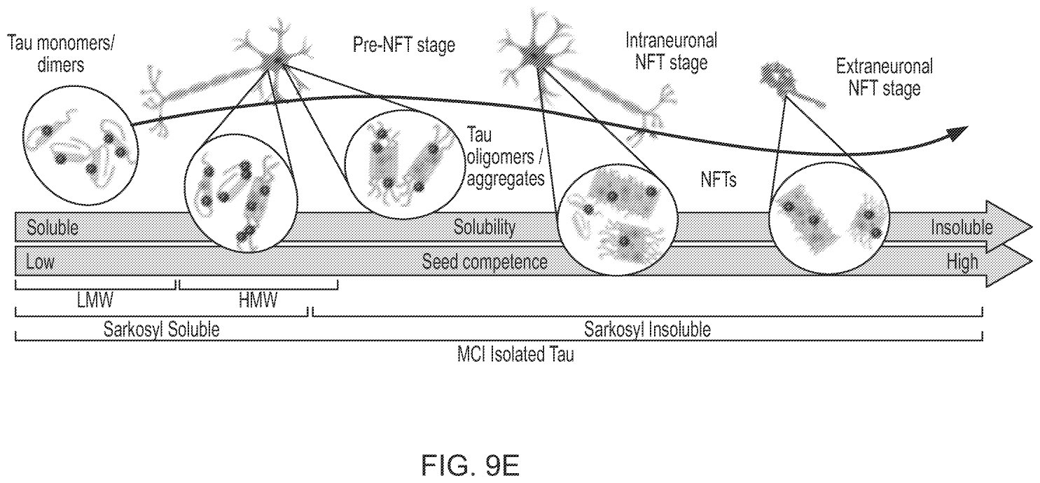

The above results from the sarkosyl insoluble (fibrillized) Tau from AD and control subjects indicated that there is processivity and an order in which PTMs occur in the fibrillization process. To examine this results further and to determine the minimum set of PTMs associated with seeding competency, using our quantitative and qualitative platform, we examined a series of different species of Tau associated with differing molecular weights and seeding capacities including size fractionated Tau from human patients (FIG. 9A) described in Takeda et al (Takeda et al., 2015). Four different Tau fractions were analyzed including: 1) sarkosyl soluble Tau (‘Soluble’), 2) MCI antibody-isolated Tau (isolated from soluble fraction by MC-1 antibody immunoaffinity columns (Jicha et al., 1999), soluble Low Molecular Weight Tau (˜50 kDa, ‘LMW’), and 4) oligomeric High Molecular Weight Tau (>120 kDa HMW); the latter two were fractionated by size exclusion chromatography (Takeda et al., 2015). The data from these four Tau fractions were compared with the data from our sarkosyl insoluble Tau isolates (fibrillar Tau; ‘insoluble’).

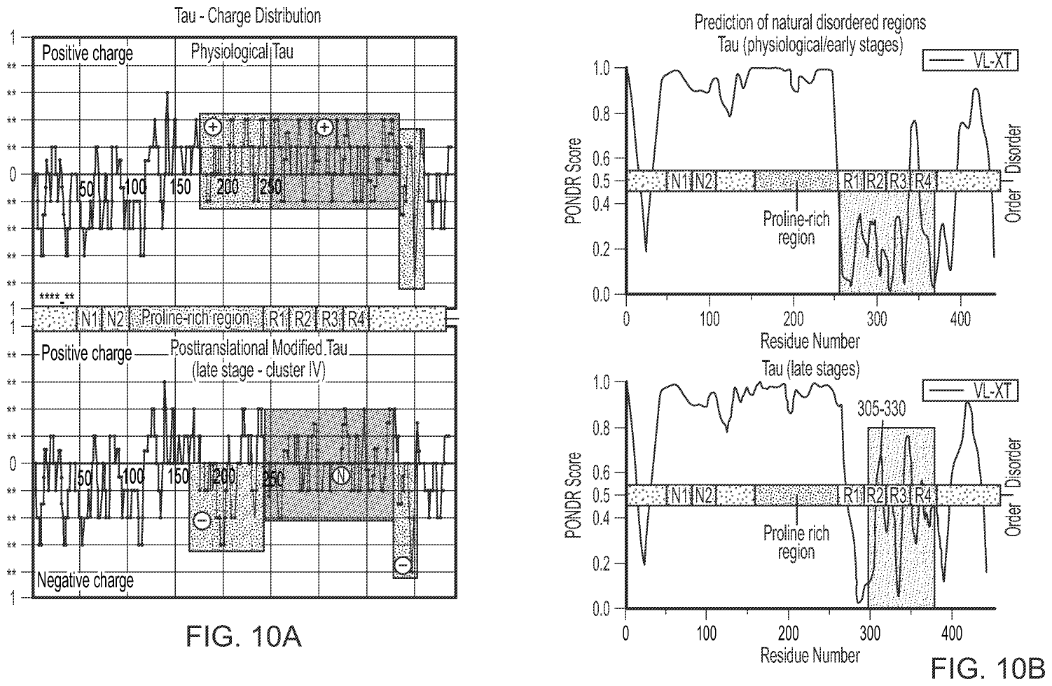

To better understand the molecular nature of the Tau species in each fraction, we compared the relative peptide quantities of the measured Tau species (FIG. 9B, FIG. 4B). When we compare the Tau profiles of the LMW and the HMW Tau species in the top left panel we observe that the unmodified intrinsically disordered domain encompassing the N-terminus to the end of the PRR is overrepresented in the LMW, which is seeding incompetent in the biosensor assay (Takeda et al., 2015), whereas the modified MBD is overrepresented in the HMW seeding competent fraction. When the sarkosyl insoluble fraction of AD (seeding competent) and control (seeding incompetent) subjects is compared in this manner we observe a similar result. The comparative analysis of the two seeding competent fractions, HMW Tau and the larger fibrillized insoluble Tau, shows a similar pattern, however the MBD is even more enriched in the insoluble Tau fraction and the peptide [260-267] is underrepresented, thus indication a higher phosphorylation stoichiometry in this peptide (S262, T263). The modification extent of Tau peptides from N- to C-terminus in each of the five fractions is shown in FIG. 9C. The comparison of the two seeding incompetent AD and control Tau sarkosyl soluble fractions shows no marked differences with both being enriched in the N-terminal regions (FIG. 9C). To summarize these quantitative data suggests that the N region of Tau is negatively correlated with the seeding capacity. In contrast, a high concentration of MBD is positively correlated with seeding activity and fibril size. In FIG. 9D, the modification profiles of the 5 described Tau species are mapped. LMW Tau shows a similar modification pattern to the Tau as the sarkosyl soluble fraction with 6 phosphorylations in the proline rich region (PRR), with T181, S199, S202, T231 being the most consistently observed sites across all monomeric fractions. The sarkosyl soluble and LMW Tau do not have positive seeding activity in biosensor assays (Holmes et al., 2014; Takeda et al., 2015). HMW Tau which is thought to be oligomeric Tau, displays 20 phosphorylation sites (6 in the N-terminus, 10 located in the PRR; and 4 in the C-terminal region) as well as 3 acetylation and 4 ubiquitin sites in the MBD. In accord with the observations in other fractions, there is notable patient to patient variability in the exact patterns of PTMs in the HMW fraction, in the context of overall similarities in tau molecules isolated with these techniques. This HMW oligomeric Tau form appears to have the minimum set of Tau PTMs associated with seeding, where we observe acetylation and ubiquitination as being unique to seeding positive Tau compared with LMW and sarkosyl soluble seeding negative fractions. A striking aspect of these modification maps which is consistent with our analysis of sarkosyl insoluble Tau is the fact that the PRR appears to accumulate phosphorylation sites and the MBD appears to accumulate acetylation and ubiquitination modifications with increasing size of Tau aggregates. Interestingly, charge neutralizing modifications such as acetylations and ubiquitination are mainly found within the core pocket [345-375] of Tau (K353, K369, K370). Disorder Prediction and Charge Calculations reveal that the MBD becomes neutralized and particularly disordered beginning from residue 305 to 350 (FIGS. 10A and 10B).

### Example 7: Stoichiometric PTM Maps of Human Pathologic Tau Identify Critical Steps in Disease Progression

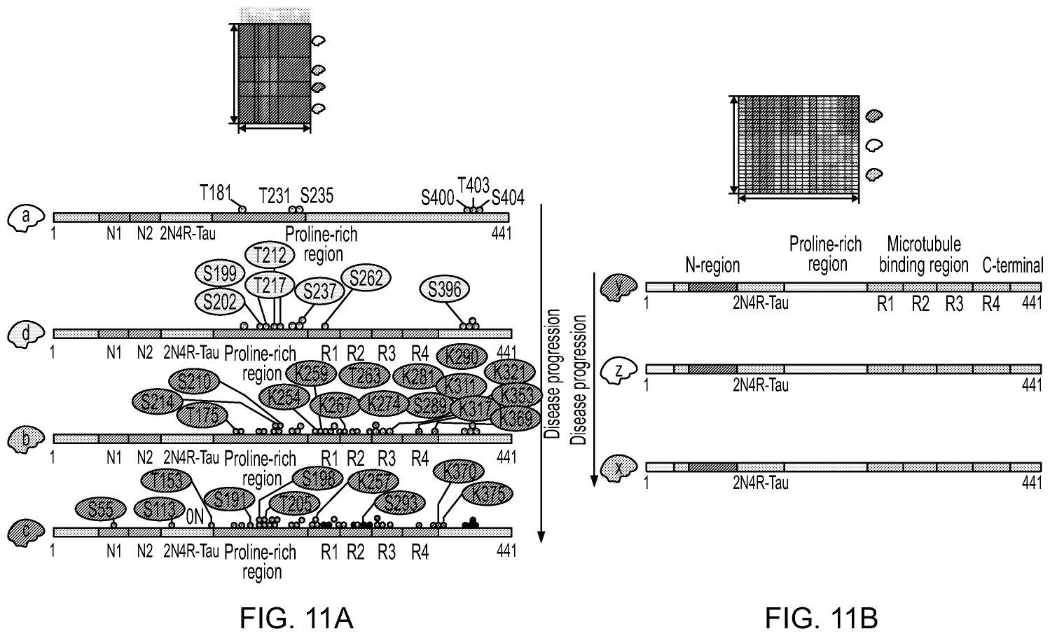

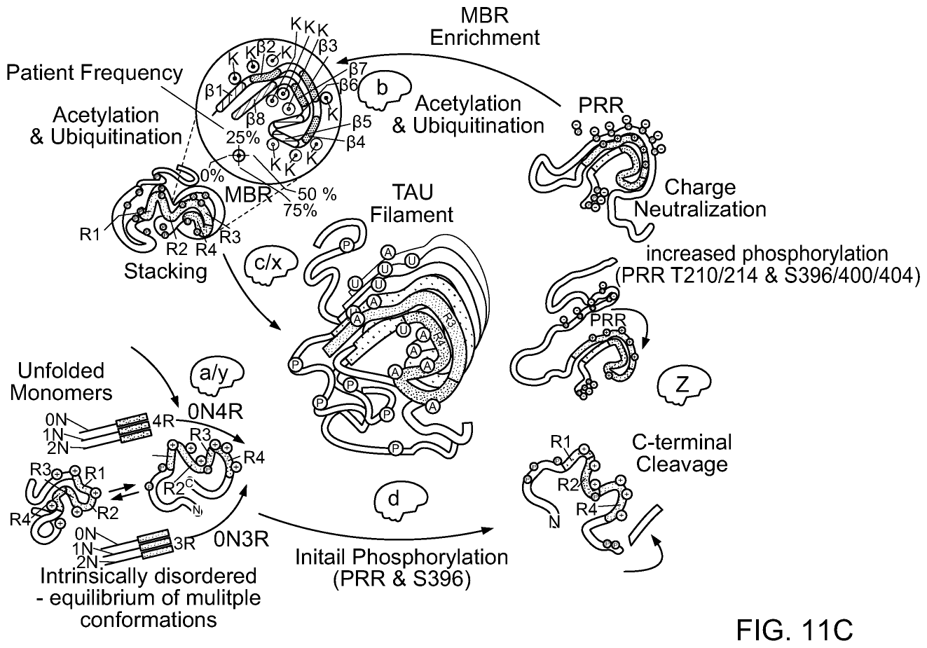

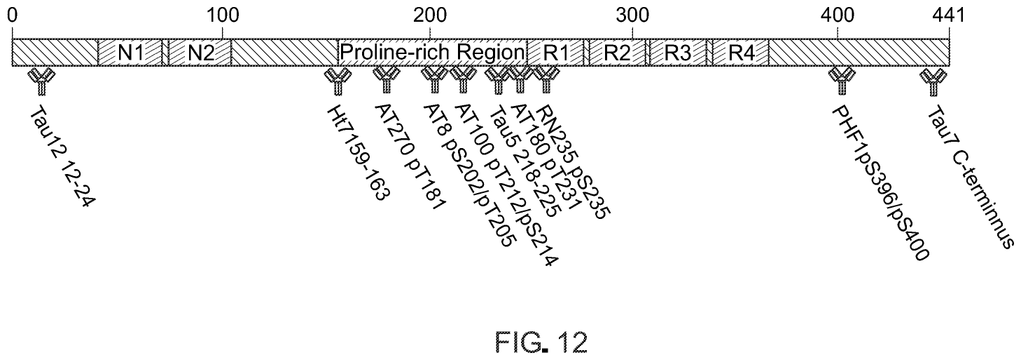

In summary of the multiple analyses performed, in FIGS. 11A and 11B we depict the most salient features observed in both the qualitative and quantitative mass spectrometry data from the pathologic Tau.

In FIG. 11A, we depict the processive nature of the Tau modification profiles suggested by the hierarchical clustering analysis. These data indicate that there is an increase in the number of PTMs and the occupancy of these PTM sites observed in different stages of disease and these provides insights into the aggregation process and disease progression. The cluster a with the lowest average Braak stage of 0-I only displayed phosphorylation of Tau (BA39) with 3 sites occupied in the PRR and another 3 in the aa [400-404]. In cluster d subjects with average Braak stage of III to IV, we observed an increase in 6 addition phosphorylation sites in the PRR domain and one in the C-terminal at S396. In cluster b with an average Braak of V or VI we observe an appearance of acetylation and ubiquitination at multiple sites in the MBD and in cluster c with the highest Braak stage of VI, we observe an overall increase in modifications from N to C-terminus.

In FIG. 11B, we summarize the FLEXITau analysis results. In general, the qualitative data agree with the quantitative data from the FLEXITau analyses. However, there are interesting observations to be made when comparing the FLEXITau data and the PTM mapping data. The FLEXITau data is important in that it identifies regions of Tau associated with pathology, for example the three peptides identified as defining AD are the MBD domain which is increased in the AD subjects, the PRR which is highly modified in disease and the endmost C-terminal peptide, which is underrepresented in its unmodified form and is explained by caspase cleavage.

In FIG. 11C, we posit a model for Tau fibril formation in AD. Our analyses reveal that 0N and 4R isoforms are predisposed to aggregation. Furthermore, a stepwise cascade of Tau modification including C-terminal cleavage, negatively charged phosphorylation in the PRR, followed by charge neutralizing acetylation and ubiquitination in the enriched MBR are progressive steps in the process of Tau fibril formation and AD disease progression.

### Example 8: PTM Profiles of Pathologic Tau

Understanding the Tau proteoforms prevalent in AD pathology and seeding is important, particularly because pharmaceutical efforts are pivoting to Tau as a therapeutic target using antibodies and small molecules. As only ˜0.1o % of all antibodies can cross the blood brain barrier (BBB), increasing the specificity and affinity of the intended therapeutic antibody towards the pathologic form of Tau is essential for a successful therapeutic. However, for the rational design of both antibody and small molecule therapeutics for Tau, a better understanding of the molecular features of the pathologic forms of Tau in human disease is needed.

Our analysis of pathological Tau from 91 subjects (49 AD patients and 42 control subjects), cumulatively identifies 95 PTMs, at 88 amino acid residues. We mapped 55 phosphorylation sites; 17 ubiquitination sites, 19 acetylation sites and four methylation sites along the length of the Tau protein. (FIG. 3E). The PTM maps reveal an extensive interplay of distinct modifications at the same amino acid residues (FIG. 3E), an observation partially consistent with the recent Tau PTM mapping results in mouse (Morris et al., 2015). Interestingly, the PTM profiles of pathologic Tau isolated from human brain tissues were heterogeneous across subjects. Analysis of the frequency of each PTM across the AD and control groups showed a striking pattern. Phosphorylation is observed with highest frequency in the PRR and the C-terminal tail, whereas acetylation and ubiquitination cluster heavily in the MBD, which has been shown to form the core region of the Tau filaments (Fitzpatrick et al., 2017). Consistent with previous biochemical studies in mice (Morris et al., 2015; Yang and Seto, 2008), most of the human Tau acetylation sites identified were alternatively ubiquitinated (FIG. 3E). These sites include three KXGS motifs, which are known to regulate neurite extension and the binding of Tau to microtubules (Biernat et al., 1993; Biernat and Mandelkow, 1999). Epitopes important in AD such as AT8 and AT180 were identified in both cohorts with high frequency. However, our data also identified other epitopes with equally high frequencies, including ubiquitination at K311 and K317 and acetylation at K369. Unsupervised analyses based on presence or absence of PTMs, clustered the subjects into 4 major groups and revealed that there were specific combinations of modifications in each of these groups. The set of modifications were largely reflective of disease progression and therefore could be associated with disease stages or different disease phenotypes. These data demonstrate that early intervention may need different therapeutics to late stage AD because there are distinct differences in PTM profiles associated with each stage of disease. Exploring the roles of these PTMs provides mechanistic insights into disease pathology and progression. Furthermore, our data provides a comprehensive PTM atlas for pathological Tau in human tissue and will be useful for future biomarker studies from systemic and proximal body fluids such as CSF, PBMC's, platelets and blood for diagnosis and will facilitate ongoing diagnostic research.

Analysis of size fractionated, seed competent and incompetent Tau species isolated from AD patients and control subjects reveals that the Tau PTM landscape as shown in FIGS. 9A-E, differs between seeding competent (high molecular weight and sarkosyl insoluble Tau) to the non-seeding competent Tau species (low-molecular weight, and sarkosyl soluble Tau). We identify the minimal set of PTMs associated with prion-like activity and our data suggests that these modifications could define novel epitopes to be targeted by therapeutic antibodies. Furthermore, the PTM landscape of these different species of Tau provide evidence that there is a progressive accumulation of PTMs as the size of the Tau species increases progressively from LMW Tau, HMW Tau, MC1 immunopurified Tau to the sarkosyl insoluble fibrillar Tau. PTMs identified in MC1 immunopurified Tau were largely identical to those in AD fibrillar Tau from the sarkosyl insoluble fraction. This data in conjunction with seeding competence studies provides the minimal set of PTMs required to form an oligomer, as mapped on the HMW Tau. Although both acetylation and ubiquitin modifications are observed in the MBD of HMW, ubiquitination appears to be exclusive to seeding-competent species. It appears that the seed competent Tau is ubiquitinated and this modification is unique to all the seeding species whereas acetylation is also observed in the seeding incompetent Tau. Tau has 19 lysine residues in the MBD of Tau, which would experience repulsive electrostatic forces within the oligomers or fibrils in the beta sheet fibril structure. To overcome these electrostatic forces and promote aggregation, charge neutralization of the MBD is required, and several models have been described in the literature (Heparin (Goedert et al., 1996), (Fichou et al., 2018), RNA or its associated granules (Kampers et al., 1996), (Zhang et al., 2017), (Dinkel et al., 2015), liquid-liquid phase separation and crowding (Wegmann et al., 2018)). Our data suggests that there are multiple modifications that contribute to charge neutralization of the MBD, including phosphorylation, acetylation, and ubiquitination. One type of modification is phosphorylation in the PRR domain that results in the accumulation of negative charges modification, which we postulate could neutralize the positive charge in the MBD much like heparin neutralizes the positive charge of this region to induce fibrillization of Tau. We also observe that the MBD accumulates charge neutralizing modifications such as acetylation and ubiquitination with increasing polymerization and with progression of disease. K311, K317 K321 and K369 ubiquitination/acetylation are charge neutralizing PTMs and thus these modifications would reduce the kinetic barriers to filament formation. The patient frequency of these modifications is high, however, the stoichiometry of these sites is ˜50%, thus every second Tau molecule in a fibril is modified. These data indicate that not every Tau molecule in the fibril is acetylated or ubiquitinated.

The disordered regions of Tau span the N-terminal and PRR. Our quantitative analyses of different Tau isoform show that 4R isoform dominates aggregates and the 2N and 1N regions of Tau are negatively correlated with seeding competent pathological Tau, whereas phosphorylation of Tau in the PRR is highly correlated with pathology. These data suggest that the 0N and 4R version of Tau once phosphorylated is no longer disordered and can adopt a structure capable of fibril formation. Thus, an alternate pathway towards fibril formation is one that involves the highly phosphorylated negatively charged PRR domain neutralizing positive charges of the MBD lysines by folding back on the MBD in a modified version of the previously described hairpin structure (Jeganathan et al., 2006) where the phosphorylated C-terminal and phosphorylated PRR domain stabilize the positive charge of the MBD.

Together our data supports a processive model (FIG. 11C) whereby 0N and 4R are predisposed to aggregation, cleavage of the C-terminal further enables fibrillization, followed by phosphorylation in the PRR domain and acetylation and ubiquitination in the MBD, which remove electrostatic repulsion of positively charged lysine residues in this region, thus promoting the fibrillization of the Tau. These data provide a guide for diagnostic criteria and therapeutic strategies for each stage of disease, for example, antibodies or small molecule therapeutics specific to each stage of disease in AD need to be developed.

### Example 9: Antibody Development for Therapeutic Approaches Against Alzheimer's Disease

### Use FLEXITau Information to Develop New Antibodies Specific to the Pathogenic Forms of Tau in Alzheimer's Disease

The Examples of the current disclosure identified novel PTMs and epitopes of Tau associated with AD pathology. In addition, Tau modifications found in human brain tissue from Tauopathy patients including Alzheimer's Disease were quantified using our FLEXITau strategy. FLEXITau data from about 130 patients were analyzed to prioritize sites as targets for therapeutic intervention. These sites were prioritized by evaluating the PTM stoichiometry, the PTM frequency, as well as specificity to AD compared with control patient tissues.

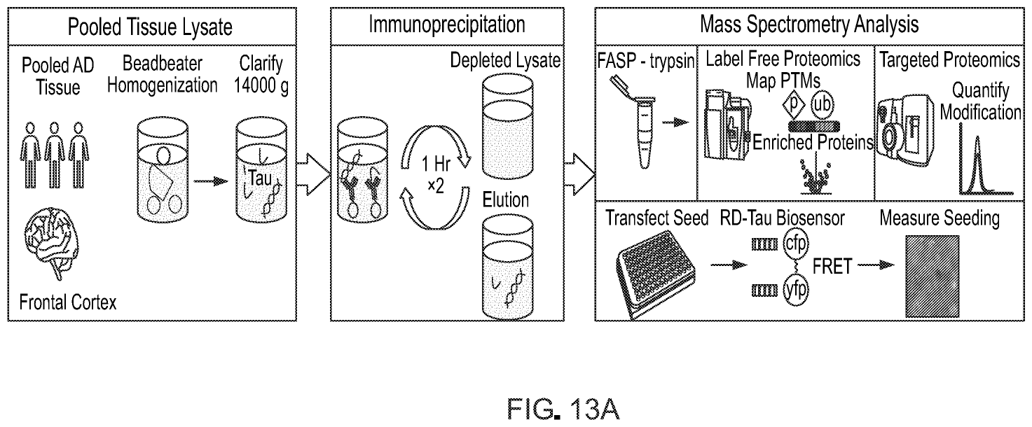

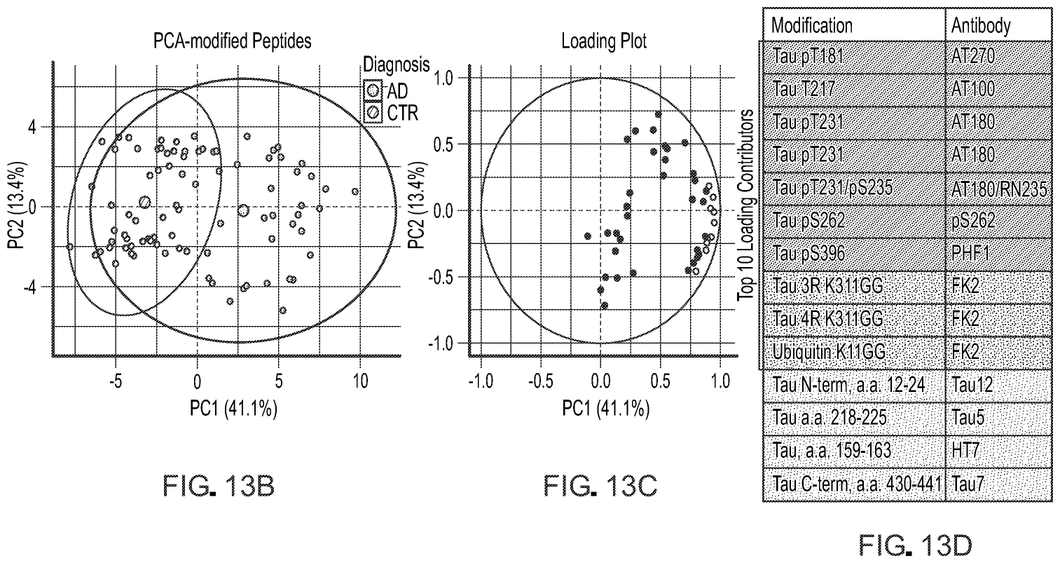

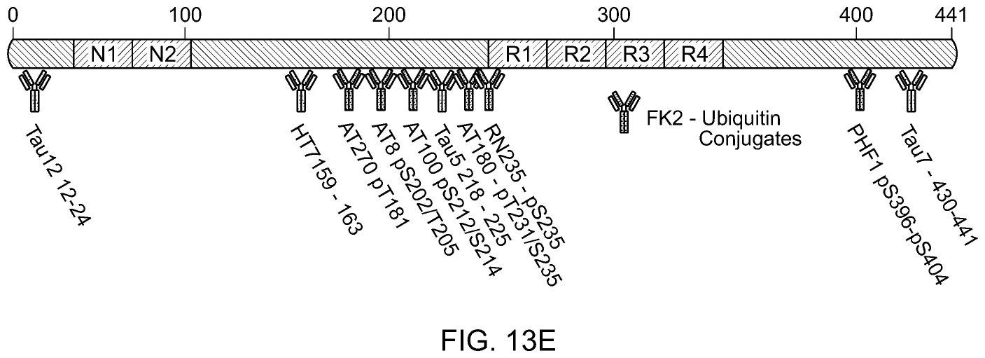

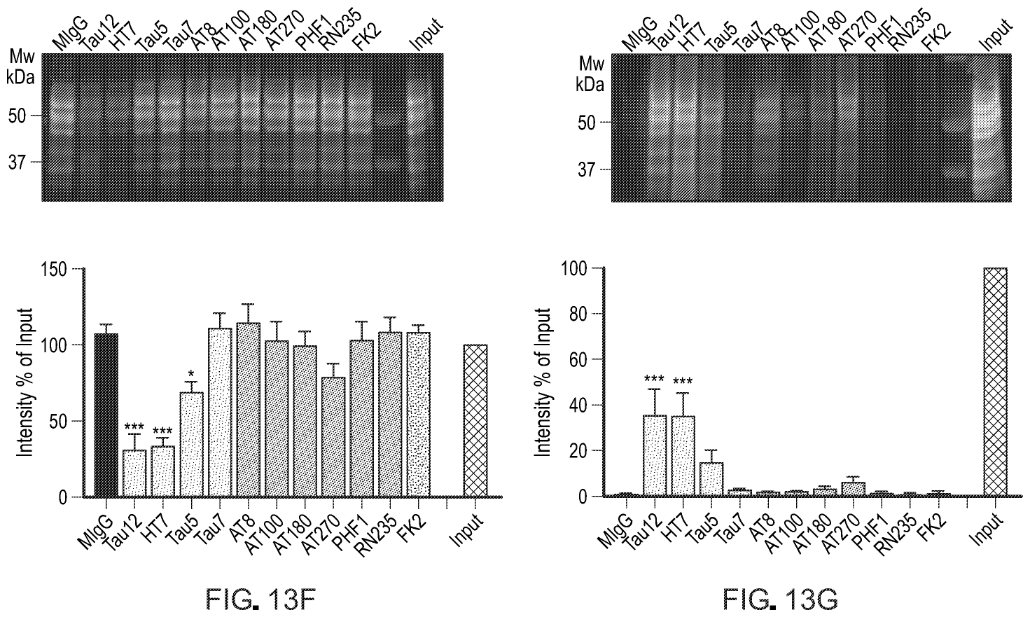

A shortlist of modified Tau target priorities was provided. This list was based on a mass spectrometry proteomics dataset with tissue from 46 of AD patients and 40 CTRL patients containing both data-dependent acquisition data on identified Tau peptides and their intensities, as well as quantitative modification stoichiometry information generated using the multiple reaction monitoring FLEXITau approach. Tau modifications for therapeutic targeting were prioritized based on the following analyses 1) A principal component analysis was performed including all peptides modified by phosphorylation, ubiquitination, and acetylation detected in at least 50% of cases in one group in the sarkosyl insoluble fraction of BA39(angular gyrus) tissues from CTRL and AD cases. Component 1 separated AD from CTRL cases, and a loading plot was then used to identify the peptides that account for this separation. (FIGS. 13A-13G) 2) Identification frequency of modifications across AD tissue cohort, with high frequency modifications given higher priority. 3) Modification extent as measured by the FLEXITau assay, with sites displaying higher modification extent being prioritized. These analyses yielded the following list of modifications:
Ubiquitination—K254, K311, K317, K311/K317
Phosphorylation—T181, T217, S235, T231/S235, S396/S400
Acetylation—K369

From this list, ubiquitination at K311, K317, K311/K317, and phosphorylation at T231/S235 were selected for initial antibody development. The characterization of seeding positive high molecular weight Tau was prioritized as these molecules can induce cell to cell seeding in vitro and in vivo models and as such would be key targets for therapeutic antibodies.

FIGS. 13D and 13E show a list of specific sites and post-translational modifications which the FLEXITau data and statistical approaches indicate are the most specific and consistent in AD, and therefore the best targets for therapeutic antibodies.

A tiered priority list (Table 5) was generated with ubiquitination at lysine 311 and/or 317 (ubK311/ubK317), as well as phosphorylation at threonine 231 and serine 235 (pT231/pS235) as primary targets. Given the need to target extracellular Tau species that propagate pathology, emphasis was placed on analyzing modifications in high molecular weight (HMW).

TABLE 5
Tau antibody prioritized sites

| Phosphorylation | Ubiquitination | Acetylation | Truncation |
|---|---|---|---|
| 1st priority | | | |
| T111/S113 | K254 | K311 | |
| T181 | K311 | K317 | |
| T181/T184 | K317 | K369 | |
| T184 | K311/K317 | | |
| T191 | | | |
| T217 | | | |
| T231/S235 | | | |
| T231/S237 | | | |
| T231/S238 | | | |
| S262 | | | |
| S396/S400 | | | |
| 2nd priority | | | |
| T212 | K257 | K343 | 241/242 |
| T212/T217 | K259 | K353 | 244/245 |
| S289 | K267 | K370 | 245/246 |
| S305 | K274 | K375 | G302/G303 |
| Y310 | K321 | | G303/G304 |
| | K343 | | G304/S305 |
| | | | N368/K369 |
| | | | Y394/K395 |
| 3rd priority | | | |
| T153 | | | |
| T175 | | | |
| S356 | | | |
| T361 | | | |

Comparison of human postmortem tissue mass spectrometry data identified AD-specific Tau PTMs to target using therapeutic antibodies, of which pT231/pS235 (This multiply phosphorylated epitope is observed earlier in disease and is only present in all the Tauopathies not in controls with high frequency and high stoichiometry) and ubK311/ubK317 (this epitope is specific for seeding positive fractions and is found in the symptomatic AD) were selected for initial antibody development campaigns.

### Develop HMW Tau Specific Antibodies Using FLEXITau Data

HMW Tau from Alzheimer's disease (AD) patient brain tissue has been shown to seed Tau aggregation and transmission from cell to cell in both cellular assays and live mouse models. The theoretical efficacy of therapeutic Tau antibodies hinges on the ability to bind and neutralize Tau species in the extracellular space during the cell-to-cell transmission process. Given the potential role of HMW Tau in cell-to-cell transmission, characterization of the modifications on these species using mass spectrometry to a) confirm/alter priorities of previously shortlisted Tau modifications and b) identify molecular features unique to HMW Tau for additional antibody development campaigns. We obtained HMW and LMW Tau isolated from AD brains, and performed mass spectrometry experiments to identify modifications on these species as well as quantify the modification extent using the FLEXITau assay. By comparing the presence/absence of modifications in seeding competent versus seeding incompetent size-resolved Tau preparations including size exclusion chromatography fractionated HMW/LMW Tau, immunoprecipitated PHF Tau, and sarkosyl insoluble Tau (FIGS. 13A-13G), an expanded target priority list was provided (Table 5)

We performed mass spectrometry analyses on the HMW weight fraction of the Tau known to seed aggregates in cellular assays. This HMW Tau appears to have a specific set of modifications that are different from other Low Molecular Weight (LMW) Tau forms. The rationale for characterizing these PTM forms of HMW Tau using our state-of-the-art FLEXITau assay is that the information obtained would allow us to specifically target the form of Tau that causes the spread of disease in patients.

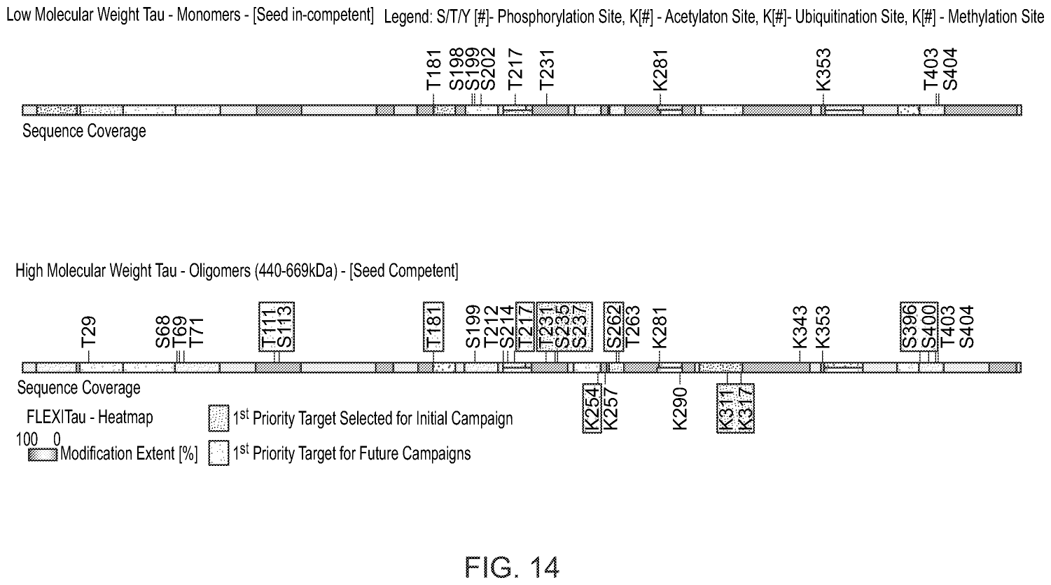

Additional HMW and LMW Tau preparations were characterized and the modifications specific to HMW Tau and the FLEXITau modification stoichiometry were provided (FIG. 14). The previously selected targets ubK311/ubK317 and pT231/pS235 were also identified in HMW, but not LMW Tau, reaffirming selection of these sites as primary targets. The target priority list (Table 5) was updated to reflect the best modifications to target in HMW Tau.

Characterization of Tau PTMs in HMW and LMW Tau confirms relevance of selected targets pT231/pS235 and ubK311/ubK317 as well as suggesting additional targets for prioritization.

### Develop a Fully Validated Mass Spectrometry-Based Quantitative Assay for the Modified Tau Peptides of Interest.

The purpose of these experiments was threefold: 1) To optimize and validate a general workflow suitable for antibody screening, 2) To get a baseline for well-characterized Tau antibodies for comparison purposes. 3) To provide additional information on the relevance of epitopes of interest to seeding.

Antibodies, such as AT8 and PHF-1 that are commercially available were used to purify Tau from human tissues and measure these forms of Tau using FLEXITau. These methods allowed us to set up biochemistry workflows that can be used to benchmark.

A set of commercial antibodies targeting sites of interest was selected for characterization. The workflow was tested with the selected antibodies, showing detectable Tau immunoprecipitation even with phospho-Tau specific antibodies. Testing of seeding of IP eluates showed that AT180, PHF1, and FK2 were most specific for seed-competent Tau (highest seeding per unit of Tau). Mass spectrometry analysis of the eluates demonstrated that the workflow was able to effectively quantify peptides for the target PTMs as well as additional Tau PTMs. Enrichment of target modified Tau peptides (i.e. pT181 for AT270) demonstrates ability of assay to measure enrichment of different populations of modified Tau. Antibodies specific for pathological Tau (AT180, PHF1, FK2) enriched Tau with a similar modification profile. Modified peptides at both target sites pT231/pS235 and ubK311/ubK317 correlated with each other in eluates suggesting they are present on the same proteoforms.

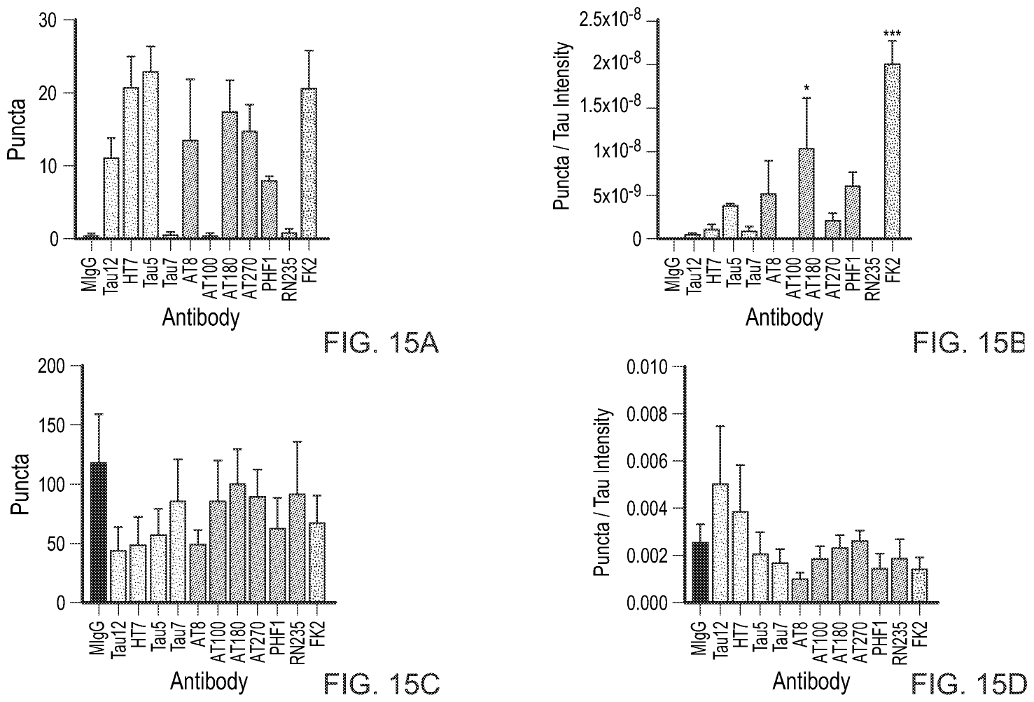

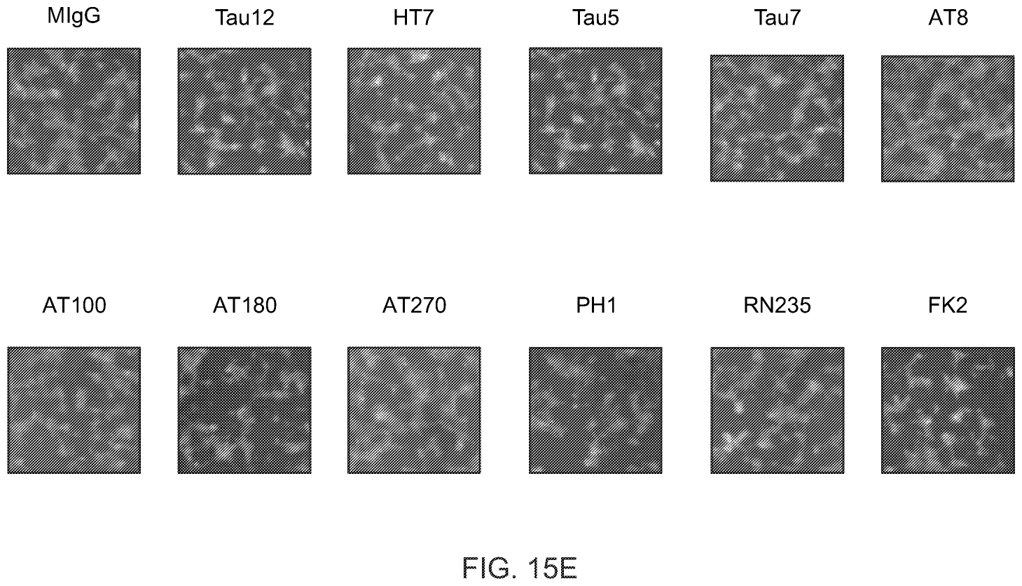

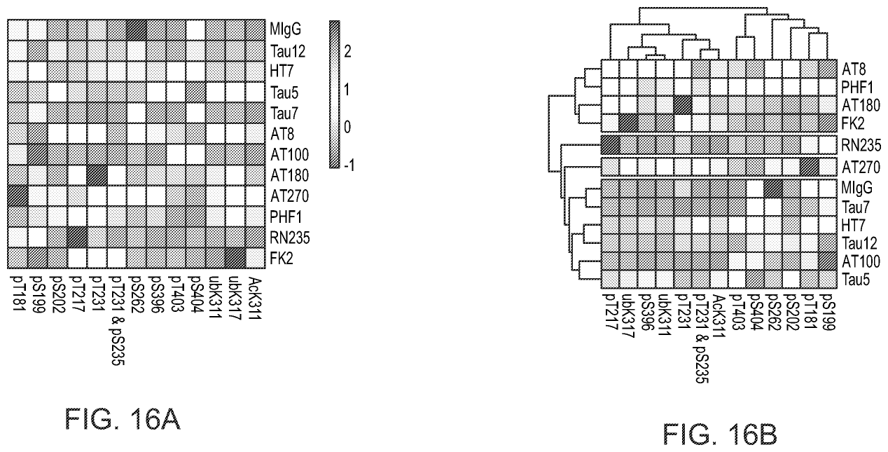

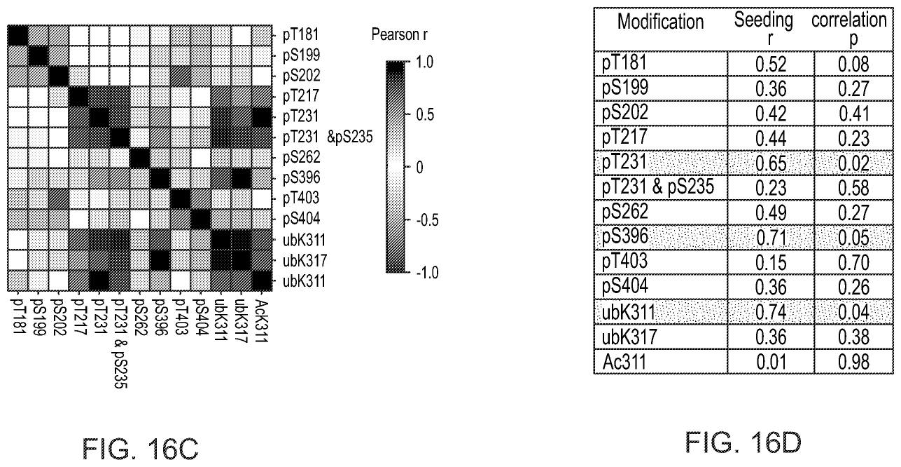

For this assay, a pooled PBS lysate of 3 frontal cortex AD tissues was generated and validated. The material showed characteristic immunoreactivity in immunoblots and seeded aggregation in HEK293 Tau-RD biosensor cells. We generated immunoblotting (FIGS. 13F and 13G), seeding (FIG. 15), and label-free mass spectrometry data (FIG. 16) for the material immunoprecipitated using the antibodies outlined in FIG. 13D. As expected, the pan-Tau antibodies effectively immunoprecipitated Tau, while the phospho-Tau and conjugated ubiquitin (FK2) showed minimal to no Tau immunoreactivity on the pan-Tau immunoblot (FIGS. 13F and 13G). To get a measure of specificity of the antibody for pathological Tau, we used the eluates and immunodepleted lysates from the IPs for seeding in the HEK293-TauRD-P301S biosensor assay and divided the seeding (counted puncta) by the amount of Tau immunoprecipitated, providing a value of puncta/intensity unit of Tau. This data showed that while pan-Tau antibodies effectively immunoprecipitated Tau, they did not enrich for seeding competent Tau. Conversely, the phospho-specific antibodies AT8, AT180, PHF1, and the conjugated ubiquitin antibody FK2 enriched for seeding competent Tau. The antibody specific for phosphorylation at pT181, AT270, did not enrich for seeding Tau species, showing that not all phospho-Tau is associated with seeding. Importantly, while not showing evidence of reduced seeding in the immunodepleted lysate, the seeding in the eluates supported selection of pT231 (AT180), and FK2 (ubK311/K317) as targets.

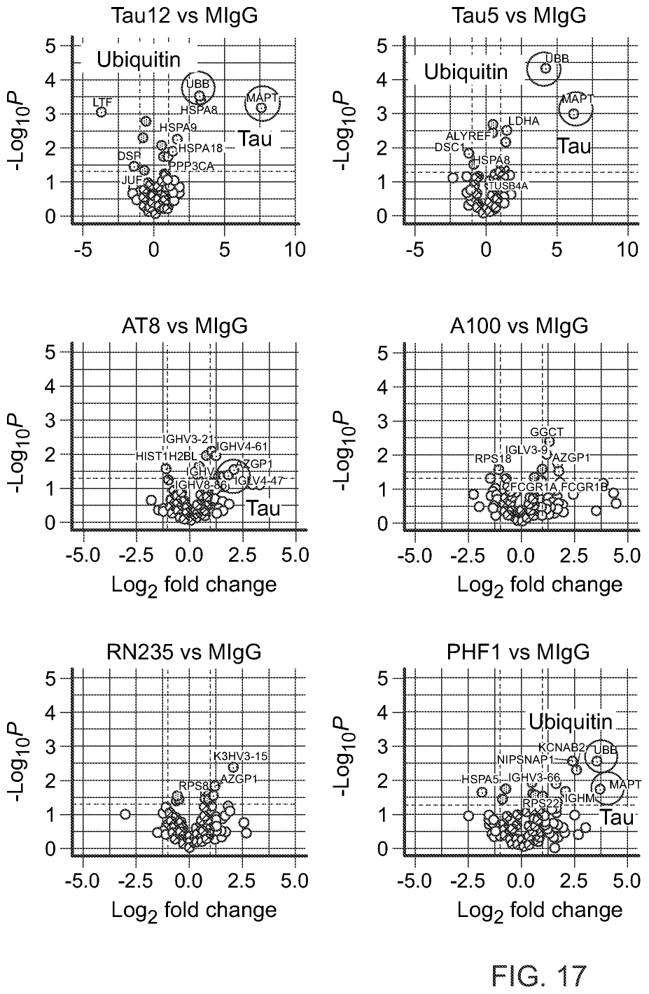

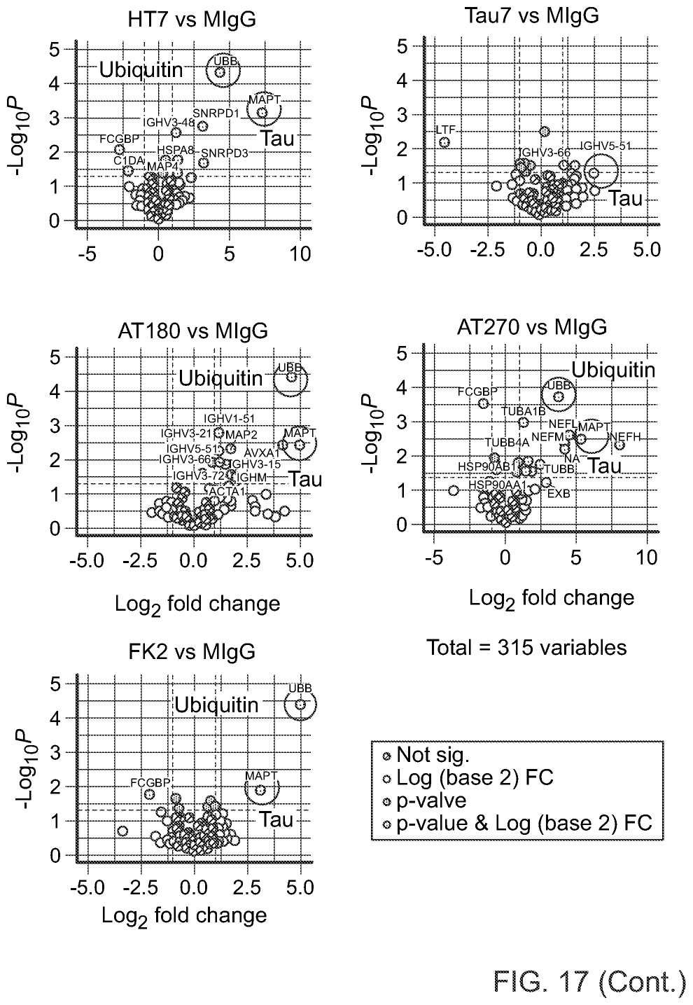

Analysis of label-free proteomics experiments identified Tau modifications that were previously identified in sarkosyl insoluble Tau and HMW Tau (FIGS. 13-14), including phosphorylation at T231 & S235 and ubiquitination on K311/K317. Importantly, enrichment of target sites for antibodies i.e. pT181 for AT270, and pT231 for AT180, and ubiquitinated Tau for FK2 were detected, demonstrating target engagement of these antibodies and the ability of workflow to test target engagement. Interestingly, relative depletion of single phosphorylated peptides was detected in the case of AT8 (S199, 5202) and PHF1 (T403, 5404), which may be explained by preferential binding to multiple phosphorylated species at this site, which were not detected in this study. Hierarchical clustering of the modified peptides resulted in one cluster containing the pan-Tau antibodies (Tau5, Tau7, Tau12, and HT7) and a second containing the phospho-Tau antibodies (AT8, AT180, AT270), and FK2. Note that the MIgG control and AT100 (which did not enrich for Tau, FIG. 17) representing non-specifically bound Tau clustered with the pan-Tau antibodies. AT270 and RN235 formed separate clusters but were more like pan-Tau than phospho-Tau in terms of modification profile. When correlations were calculated between the intensities of different modified peptides, pT217, pT231, pT231/S235, ubK311, ubK317, and AcK311, all clustered together, suggesting they are present on overlapping proteoform populations. Additionally, correlations between modified peptide intensities and seeding were calculated, showing significant correlations between phosphorylation at pT231 and S396, as well as ubiquitination at K311.

We then analyzed relative enrichment of proteins in the IP eluate for each of the antibodies vs MIgG control (FIG. 17). This analysis showed that for all the antibodies except for AT100 and RN235 there was enrichment of Tau. Ubiquitin was enriched in all Ips except AT100, RN235, AT8, and Tau7, but with different stoichiometries. FK2, AT180, and PHF1 had the highest ubiquitin to Tau ratio, suggesting they pull down more ubiquitinated Tau.

These data show that antibody characterization workflow can effectively quantify the modified peptides of interest and show evidence of an overlapping population of seeding Tau species.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Abreha, M. H., Dammer, E. B., Ping, L., Zhang, T., Duong, D. M., Gearing, M., Lah, J. J., Levey, A. I., and Seyfried, N. T. (2018). Quantitative Analysis of the Brain Ubiquitylome in Alzheimer's Disease. Proteomics 18, e1800108.
Adams, S. J., DeTure, M. A., McBride, M., Dickson, D. W., and Petrucelli, L. (2010). Three repeat isoforms of tau inhibit assembly of four repeat tau filaments. PLoS One 5, e10810.
Aebersold, R., Agar, J. N., Amster, I. J., Baker, M. S., Bertozzi, C. R., Boja, E. S., Costello, C. E., Cravatt, B. F., Fenselau, C., Garcia, B. A., et al. (2018). How many human proteoforms are there?Nat Chem Biol 14, 206-214.
Alonso, A., Zaidi, T., Novak, M., Grundke-Iqbal, I., and Iqbal, K. (2001). Hyperphosphorylation induces self-assembly of tau into tangles of paired helical filaments/straight filaments. Proc Natl Acad Sci USA 98, 6923-6928.
Arendt, T., Stieler, J. T., and Holzer, M. (2016). Tau and tauopathies. Brain Res Bull 126, 238-292.
Arriagada, P. V., Marzloff, K., and Hyman, B. T. (1992). Distribution of Alzheimer-type pathologic changes in nondemented elderly individuals matches the pattern in Alzheimer's disease. Neurology 42, 1681-1688.
Bierer, L. M., Hof, P. R., Purohit, D. P., Carlin, L., Schmeidler, J., Davis, K. L., and Perl, D. P. (1995). Neocortical neurofibrillary tangles correlate with dementia severity in Alzheimer's disease. Arch Neurol 52, 81-88.
Biernat, J., Gustke, N., Drewes, G., Mandelkow, E. M., and Mandelkow, E. (1993). Phosphorylation of Ser262 strongly reduces binding of tau to microtubules: distinction between PHF-like immunoreactivity and microtubule binding. Neuron 11, 153-163.
Biernat, J., and Mandelkow, E. M. (1999). The development of cell processes induced by tau protein requires phosphorylation of serine 262 and 356 in the repeat domain and is inhibited by phosphorylation in the proline-rich domains. Mol Biol Cell 10, 727-740.
Braak, H., and Braak, E. (1991). Neuropathological stageing of Alzheimer-related changes. Acta Neuropathol 82, 239-259.
Bramblett, G. T., Goedert, M., Jakes, R., Merrick, S. E., Trojanowski, J. Q., and Lee, V. M. (1993). Abnormal tau phosphorylation at Ser396 in Alzheimer's disease recapitulates development and contributes to reduced microtubule binding. Neuron 10, 1089-1099.
Brosch, J. R., Farlow, M. R., Risacher, S. L., and Apostolova, L. G. (2017). Tau Imaging in Alzheimer's Disease Diagnosis and Clinical Trials. Neurotherapeutics 14, 62-68.
Clavaguera, F., Lavenir, I., Falcon, B., Frank, S., Goedert, M., and Tolnay, M. (2013). “Prion-like” templated misfolding in tauopathies. Brain Pathol 23, 342-349.
Cleveland, D. W., Hwo, S. Y., and Kirschner, M. W. (1977). Physical and chemical properties of purified tau factor and the role of tau in microtubule assembly. J Mol Biol 116, 227-247.
Cohen, T. J., Guo, J. L., Hurtado, D. E., Kwong, L. K., Mills, I. P., Trojanowski, J. Q., and Lee, V. M. (2011). The acetylation of tau inhibits its function and promotes pathological tau aggregation. Nat Commun 2, 252.
Crowther, T., Goedert, M., and Wischik, C. M. (1989). The repeat region of microtubule-associated protein tau forms part of the core of the paired helical filament of Alzheimer's disease. Ann Med 21, 127-132.
de Calignon, A., Polydoro, M., Suarez-Calvet, M., William, C., Adamowicz, D. H., Kopeikina, K. J., Pitstick, R., Sahara, N., Ashe, K. H., Carlson, G. A., et al. (2012). Propagation of tau pathology in a model of early Alzheimer's disease. Neuron 73, 685-697.
Dinkel, P. D., Holden, M. R., Matin, N., and Margittai, M. (2015). RNA Binds to Tau Fibrils and Sustains Template-Assisted Growth. Biochemistry 54, 4731-4740.
Falcon, B., Zhang, W., Schweighauser, M., Murzin, A. G., Vidal, R., Garringer, H. J., Ghetti, B., Scheres, S. H. W., and Goedert, M. (2018). Tau filaments from multiple cases of sporadic and inherited Alzheimer's disease adopt a common fold. Acta Neuropathol 136, 699-708.
Fichou, Y., Lin, Y., Rauch, J. N., Vigers, M., Zeng, Z., Srivastava, M., Keller, T. J., Freed, J. H., Kosik, K. S., and Han, S. (2018). Cofactors are essential constituents of stable and seeding-active tau fibrils. Proc Natl Acad Sci USA 115, 13234-13239.
Fitzpatrick, A. W. P., Falcon, B., He, S., Murzin, A. G., Murshudov, G., Garringer, H. J., Crowther, R. A., Ghetti, B., Goedert, M., and Scheres, S. H. W. (2017). Cryo-EM structures of tau filaments from Alzheimer's disease. Nature 547, 185-190.
Giannakopoulos, P., Herrmann, F. R., Bussiere, T., Bouras, C., Kovari, E., Perl, D. P., Morrison, J. H., Gold, G., and Hof, P. R. (2003). Tangle and neuron numbers, but not amyloid load, predict cognitive status in Alzheimer's disease. Neurology 60, 1495-1500.
Glenner, G. G., and Wong, C. W. (1984). Alzheimer's disease: initial report of the purification and characterization of a novel cerebrovascular amyloid protein. Biochem Biophys Res Commun 120, 885-890.
Goedert, M., Jakes, R., Spillantini, M. G., Hasegawa, M., Smith, M. J., and Crowther, R. A. (1996). Assembly of microtubule-associated protein tau into Alzheimer-like filaments induced by sulphated glycosaminoglycans. Nature 383, 550-553.
Goedert, M., Jakes, R., and Vanmechelen, E. (1995). Monoclonal antibody AT8 recognises tau protein phosphorylated at both serine 202 and threonine 205. Neurosci Lett 189, 167-169.
Gomez-Isla, T., Hollister, R., West, H., Mui, S., Growdon, J. H., Petersen, R. C., Parisi, J. E., and Hyman, B. T. (1997). Neuronal loss correlates with but exceeds neurofibrillary tangles in Alzheimer's disease. Ann Neurol 41, 17-24.
Grundke-Iqbal, I., Iqbal, K., Quinlan, M., Tung, Y. C., Zaidi, M. S., and Wisniewski, H. M. (1986). Microtubule-associated protein tau. A component of Alzheimer paired helical filaments. J Biol Chem 261, 6084-6089.
Guo, T., Noble, W., and Hanger, D. P. (2017). Roles of tau protein in health and disease. Acta Neuropathol 133, 665-704.
Hanger, D. P., Byers, H. L., Wray, S., Leung, K. Y., Saxton, M. J., Seereeram, A., Reynolds, C. H., Ward, M. A., and Anderton, B. H. (2007). Novel phosphorylation sites in tau from Alzheimer brain support a role for casein kinase 1 in disease pathogenesis. J Biol Chem 282, 23645-23654.
Hasegawa, M., Morishima-Kawashima, M., Takio, K., Suzuki, M., Titani, K., and Ihara, Y. (1992). Protein sequence and mass spectrometric analyses of tau in the Alzheimer's disease brain. J Biol Chem 267, 17047-17054.
Holmes, B. B., Furman, J. L., Mahan, T. E., Yamasaki, T. R., Mirbaha, H., Eades, W. C., Belaygorod, L., Cairns, N. J., Holtzman, D. M., and Diamond, M. I. (2014). Proteopathic tau seeding predicts tauopathy in vivo. Proc Natl Acad Sci USA 111, E4376-4385.
Jackson, S. J., Kerridge, C., Cooper, J., Cavallini, A., Falcon, B., Cella, C. V., Landi, A., Szekeres, P. G., Murray, T. K., Ahmed, Z., et al. (2016). Short Fibrils Constitute the Major Species of Seed-Competent Tau in the Brains of Mice Transgenic for Human P301S Tau. J Neurosci 36, 762-772.
Janelidze, S., Mattsson, N., Palmqvist, S., Smith, R., Beach, T. G., Serrano, G. E., Chai, X., Proctor, N. K., Eichenlaub, U., Zetterberg, H., et al. (2020). Plasma P-tau181 in Alzheimer's disease: relationship to other biomarkers, differential diagnosis, neuropathology and longitudinal progression to Alzheimer's dementia. Nat Med 26, 379-386.
Jeganathan, S., von Bergen, M., Brutlach, H., Steinhoff, H. J., and Mandelkow, E. (2006). Global hairpin folding of tau in solution. Biochemistry 45, 2283-2293.
Jicha, G. A., Weaver, C., Lane, E., Vianna, C., Kress, Y., Rockwood, J., and Davies, P. (1999). cAMP-dependent protein kinase phosphorylations on tau in Alzheimer's disease. J Neurosci 19, 7486-7494.
Jucker, M., and Walker, L. C. (2013). Self-propagation of pathogenic protein aggregates in neurodegenerative diseases. Nature 501, 45-51.
Kampers, T., Friedhoff, P., Biernat, J., Mandelkow, E. M., and Mandelkow, E. (1996). RNA stimulates aggregation of microtubule-associated protein tau into Alzheimer-like paired helical filaments. FEBS Lett 399, 344-349.
Kosik, K. S., Joachim, C. L., and Selkoe, D. J. (1986). Microtubule-associated protein tau (tau) is a major antigenic component of paired helical filaments in Alzheimer disease. Proc Natl Acad Sci USA 83, 4044-4048.
Lovestone, S., and Reynolds, C. H. (1997). The phosphorylation of tau: a critical stage in neurodevelopment and neurodegenerative processes. Neuroscience 78, 309-324.
Mair, W., Muntel, J., Tepper, K., Tang, S., Biernat, J., Seeley, W. W., Kosik, K. S., Mandelkow, E., Steen, H., and Steen, J. A. (2016). FLEXITau: Quantifying Post-translational Modifications of Tau Protein in Vitro and in Human Disease. Anal Chem 88, 3704-3714.
Martin, L., Latypova, X., Wilson, C. M., Magnaudeix, A., Perrin, M. L., and Terro, F. (2013a). Tau protein phosphatases in Alzheimer's disease: the leading role of PP2A. Ageing Res Rev 12, 39-49.
Martin, L., Latypova, X., Wilson, C. M., Magnaudeix, A., Perrin, M. L., Yardin, C., and Terro, F. (2013b). Tau protein kinases: involvement in Alzheimer's disease. Ageing Res Rev 12, 289-309.
Masters, C. L., Simms, G., Weinman, N. A., Multhaup, G., McDonald, B. L., and Beyreuther, K. (1985). Amyloid plaque core protein in Alzheimer disease and Down syndrome. Proc Natl Acad Sci USA 82, 4245-4249.
Min, S. W., Chen, X., Tracy, T. E., Li, Y., Zhou, Y., Wang, C., Shirakawa, K., Minami, S. S., Defensor, E., Mok, S. A., et al. (2015). Critical role of acetylation in tau-mediated neurodegeneration and cognitive deficits. Nat Med 21, 1154-1162.
Min, S. W., Cho, S. H., Zhou, Y., Schroeder, S., Haroutunian, V., Seeley, W. W., Huang, E. J., Shen, Y., Masliah, E., Mukherjee, C., et al. (2010). Acetylation of tau inhibits its degradation and contributes to tauopathy. Neuron 67, 953-966.
Mirra, S. S., Heyman, A., McKeel, D., Sumi, S. M., Crain, B. J., Brownlee, L. M., Vogel, F. S., Hughes, J. P., van Belle, G., and Berg, L. (1991). The Consortium to Establish a Registry for Alzheimer's Disease (CERAD). Part II. Standardization of the neuropathologic assessment of Alzheimer's disease. Neurology 41, 479-486.
Montine, T. J., Monsell, S. E., Beach, T. G., Bigio, E. H., Bu, Y., Cairns, N. J., Frosch, M., Henriksen, J., Kofler, J., Kukull, W. A., et al. (2016). Multisite assessment of NIA-AA guidelines for the neuropathologic evaluation of Alzheimer's disease. Alzheimers Dement 12, 164-169.
Morishima-Kawashima, M., Hasegawa, M., Takio, K., Suzuki, M., Yoshida, H., Titani, K., and Ihara, Y. (1995). Proline-directed and non-proline-directed phosphorylation of PHF-tau. J Biol Chem 270, 823-829.
Morris, M., Knudsen, G. M., Maeda, S., Trinidad, J. C., Ioanoviciu, A., Burlingame, A. L., and Mucke, L. (2015). Tau post-translational modifications in wild-type and human amyloid precursor protein transgenic mice. Nat Neurosci 18, 1183-1189.
Mudher, A., Colin, M., Dujardin, S., Medina, M., Dewachter, I., Alavi Naini, S. M., Mandelkow, E. M., Mandelkow, E., Buee, L., Goedert, M., et al. (2017). What is the evidence that tau pathology spreads through prion-like propagation?Acta Neuropathol Commun 5, 99.
Poorkaj, P., Bird, T. D., Wijsman, E., Nemens, E., Garruto, R. M., Anderson, L., Andreadis, A., Wiederholt, W. C., Raskind, M., and Schellenberg, G. D. (1998). Tau is a candidate gene for chromosome 17 frontotemporal dementia. Ann Neurol 43, 815-825.
Sigurdsson, E. M. (2018). Tau Immunotherapies for Alzheimer's Disease and Related Tauopathies: Progress and Potential Pitfalls. J Alzheimers Dis 64, S555-S565.
Singh, S. A., Winter, D., Kirchner, M., Chauhan, R., Ahmed, S., Ozlu, N., Tzur, A., Steen, J. A., and Steen, H. (2014). Co-regulation proteomics reveals substrates and mechanisms of APC/C-dependent degradation. EMBO J 33, 385-399.
Smith, L. M., Kelleher, N. L., and Consortium for Top Down, P. (2013). Proteoform: a single term describing protein complexity. Nat Methods 10, 186-187.
Soria, P. S., McGary, K. L., and Rokas, A. (2014). Functional divergence for every paralog. Mol Biol Evol 31, 984-992.
Spillantini, M. G., Murrell, J. R., Goedert, M., Farlow, M. R., Klug, A., and Ghetti, B. (1998). Mutation in the tau gene in familial multiple system tauopathy with presenile dementia. Proc Natl Acad Sci USA 95, 7737-7741.
Takeda, S., Wegmann, S., Cho, H., DeVos, S. L., Commins, C., Roe, A. D., Nicholls, S. B., Carlson, G. A., Pitstick, R., Nobuhara, C. K., et al. (2015). Neuronal uptake and propagation of a rare phosphorylated high-molecular-weight tau derived from Alzheimer's disease brain. Nat Commun 6, 8490.
Thijssen, E. H., La Joie, R., Wolf, A., Strom, A., Wang, P., Iaccarino, L., Bourakova, V., Cobigo, Y., Heuer, H., Spina, S., et al. (2020). Diagnostic value of plasma phosphorylated tau181 in Alzheimer's disease and frontotemporal lobar degeneration. Nat Med 26, 387-397.
Tramutola, A., Sharma, N., Barone, E., Lanzillotta, C., Castellani, A., Iavarone, F., Vincenzoni, F., Castagnola, M., Butterfield, D. A., Gaetani, S., et al. (2018). Proteomic identification of altered protein O-GlcNAcylation in a triple transgenic mouse model of Alzheimer's disease. Biochim Biophys Acta Mol Basis Dis 1864, 3309-3321.
Walker, L. C., Diamond, M. I., Duff, K. E., and Hyman, B. T. (2013). Mechanisms of protein seeding in neurodegenerative diseases. JAMA Neurol 70, 304-310.
Wang, Y., and Mandelkow, E. (2016). Tau in physiology and pathology. Nat Rev Neurosci 17, 5-21.
Wegmann, S., Eftekharzadeh, B., Tepper, K., Zoltowska, K. M., Bennett, R. E., Dujardin, S., Laskowski, P. R., MacKenzie, D., Kamath, T., Commins, C., et al. (2018). Tau protein liquid-liquid phase separation can initiate tau aggregation. EMBO J 37.
Wesseling, H. et al. (2020). Tau PTM Profiles Identify Patient Heterogeneity and Stages of Alzheimer's Disease. Cell, doi:10.1016/j.cell.2020.10.029 Wood, J. G., Mirra, S. S., Pollock, N. J., and Binder, L. I. (1986). Neurofibrillary tangles of Alzheimer disease share antigenic determinants with the axonal microtubule-associated protein tau (tau). Proc Natl Acad Sci USA 83, 4040-4043.
Yang, X. J., and Seto, E. (2008). Lysine acetylation: codified crosstalk with other posttranslational modifications. Mol Cell 31, 449-461.
Zhang, X., Lin, Y., Eschmann, N. A., Zhou, H., Rauch, J. N., Hernandez, I., Guzman, E., Kosik, K. S., and Han, S. (2017). RNA stores tau reversibly in complex coacervates. PLoS Biol 15, e2002183.

1. A method for diagnosing a tauopathy in a subject, the method comprising:
(a) obtaining a biological sample from the subject;
(b) identifying one or more post translational modifications (PTMs) in a tau protein, wherein the one or more PTMs are at positions selected from the group consisting of K24, Y29, T30, T39, K44, S46, S56, S61, S64, K67, S68, T69, T71, K87, T102, T111, S113, T153, T175, K180, T181, S184, S185, S191, S198, S199, S202, T205, S210, T212, S214, T217, T220, T231, S235, S237, S238, K240, S241, K254, K257, S258, K259, S262, T263, K267, K274, K280, K281, S289, K290, S293, K298, S305, Y310, K311, S316, K317, K321, K331, K343, K347, S352, K353, S356, T361, K369, K370, K375, K385, T386, Y394, K395, S396, S400, T403, S404, R406, S409, S412, S413, T414, S416, S422, S433, S435, K436, and K438 (based on numbering on human 2N4R isoform),
thereby diagnosing the tauopathy in the subject.

2. The method of claim 1, wherein the post-translational modification is phosphorylation, glycosylation, glycation, prolyl-isomerization, cleavage or truncation, nitration, polyamination, ubiquitination, acetylation, methylation, dimethylation, trimethylation or sumoylation.

3. The method of claim 1, wherein the subject has an overall higher level of PTMs at the one or more PTM positions as compared to a control level.

4. The method of claim 1, wherein the one or more PTM is selected from the group consisting of:
phosphorylation at one or more positions selected from the group consisting of Y29, T30, T39, S46, S56, S68, T69, T71, T102, T111, S113, T153, T175, T181, S184, S185, S191, S198, S199, S202, T205, S210, T212, S214, T217, T220, T231, S235, S237, S238, S241, S258, S262, T263, S289, S293, S305, Y310, S316, S352, S356, T361, T386, Y394, S396, S400, T403, S404, S409, S412, S413, T414, S422, S433, and S435;
acetylation at one or more positions selected from the group consisting of K24, K44, K240, K267, K274, K280, K281, K298, K311, K317, K331, K343, K347, K353, K369, K370, K375, K385, and K395;
ubiquitination at one or more positions selected from the group consisting of K180, K240, K254, K257, K259, K267, K274, K281, K290, K298, K311, K317, K321, K343, K353, K369, and K395; and
methylation at one or more positions selected from the group consisting of K67, K87, R406, and K438 (all numbering based on human 2N4R isoform).

5. The method of claim 4, wherein the one or more PTMs comprise ubiquitination at K311 and K317.

6. The method of claim 4, wherein the one or more PTMs comprise phosphorylation at T217 and S262.

7. The method of claim 1, wherein the one or more PTMs are located at the proline-rich region (PRR).

8. The method of claim 1, wherein the one or more PTMs are located at amino acid residues 212-221 of a tau protein.

9. (canceled)

10. The method of claim 1, wherein the one or more PTMs are located at a region that is C-terminus relative to the N-region of the tau protein.

11. The method of claim 1, wherein the one or more PTMs are located at amino acid residues 354-369 of a tau protein.

12. The method of claim 1, wherein the one or more PTMs are located at amino acid residues 407-437 of a tau protein.

13. The method of claim 1, wherein the one or more PTMs comprise phosphorylation at one or more positions selected from the group consisting of S199, S202 and T205.

14. The method of claim 1, wherein the one or more PTMs comprise phosphorylation at one or more positions selected from the group consisting of S198, S199, S202, and T205.

15. The method of claim 1, wherein the one or more PTMs comprise phosphorylation at one or more positions selected from the group consisting of S212, S214, and S217.

16. The method of claim 1, wherein the one or more PTMs comprise phosphorylation at T181 and/or T231.

17. The method of claim 1, wherein the one or more PTMs comprise acetylation at one or more positions selected from the group consisting of K353, K369, K370, and K375.

18. (canceled)

19. The method of claim 1, wherein the biological sample is brain tissue, plasma, or cerebrospinal fluid (CSF).

20-25. (canceled)

26. The method of claim 1, wherein the tauopathy is Alzheimer's disease (AD).

27-43. (canceled)

44. A method for treating a tauopathy in a subject, the method comprising:
(a) obtaining a biological sample from the subject;
(b) identifying one or more post translational modification (PTM) associated with one or more tau peptide fragments of a tau protein in the biological sample, wherein the one or more PTM is located on the position selected from the positions listed in Table 1;
(c) administering an effective amount of a therapeutic agent that targets the one or more tau peptide fragments,
thereby treating the tauopathy.

45-61. (canceled)

62. A method for determining stage of a tauopathy in a subject, the method comprising:
(a) obtaining a biological sample from the subject;
(b) identifying one or more post translational modification (PTM) associated with one or more tau peptide fragments of a tau protein in the biological sample, wherein the one or more PTMs are located at positions selected from:
(i) one or more PTMs in S210, S289, K274 (in 3R isoform of tau protein), K321, S305, K311 (in 3R isoform of tau protein);
(ii) one or more PTMs in T175, S237, and K281;
(iii) one or more PTMs in S235, T181, T231, T403, S404, S400, S262, T217, S396, and T212;
(iv) one or more PTMs in S214, K311, K353, K267, K259, K317 (in 3R isoform of tau protein), K311 (in 3R isoform of tau protein), K311, K317, K254, K369, S262, and T263; and
(v) one or more PTMs in S199 and S202.

63-73. (canceled)
